SEQUENCE LISTING (38 sequences)

SEQ ID NO: 1 - protein, 37 aa, Artificial Sequence
  GIPR antagonist GIP peptide
  MISC_FEATURE (10)..(10): Xaa = 2-Aminoisobutyric acid
  MISC_FEATURE (12)..(12): Xaa = Norleucine
EGTFISEYSXAXEKIKQQDFVEWLLAQKPSSGAPPPS

SEQ ID NO: 2 - protein, 37 aa, Artificial Sequence
  GIPR antagonist GIP peptide
  MISC_FEATURE (11)..(11): Xaa = 2-Aminoisobutyric acid
EGTFISEYSIXMEKIKQQDFVEWLLAQKPSSGAPPPS

SEQ ID NO: 3 - protein, 37 aa, Artificial Sequence
  GIPR antagonist GIP peptide
  MISC_FEATURE (11)..(11): Xaa = 2-Aminoisobutyric acid
EGTFISDYSIXMDKIKQQDFVEWLLAQKPSSGAPPPS

SEQ ID NO: 4 - protein, 37 aa, Artificial Sequence
  GIPR antagonist GIP peptide
  MISC_FEATURE (11)..(11): Xaa = 2-Aminoisobutyric acid
EGTFISDYSIXLDKIKQQDFVEWLLAQKPSSGAPPPS

SEQ ID NO: 5 - protein, 37 aa, Artificial Sequence
  GIPR antagonist GIP peptide
  MISC_FEATURE (11)..(11): Xaa = 2-Aminoisobutyric acid
  MISC_FEATURE (12)..(12): Xaa = Norleucine
EGTFISDYSIXXDKIKQQDFVEWLLAQKPSSGAPPPS

SEQ ID NO: 6 - protein, 37 aa, Artificial Sequence
  GIPR antagonist GIP peptide
  MISC_FEATURE (11)..(11): Xaa = 2-Aminoisobutyric acid
EGTFISEYSIXLEKIKQQEFVEWLLAQKPSSGAPPPS

SEQ ID NO: 7 - protein, 37 aa, Artificial Sequence
  GIPR antagonist GIP peptide
  MISC_FEATURE (11)..(11): Xaa = 2-Aminoisobutyric acid
  MISC_FEATURE (12)..(12): Xaa = Norleucine
EGTFISEYSIXXEKIKQQEFVEWLLAQKPSSGAPPPS

SEQ ID NO: 8 - protein, 37 aa, Artificial Sequence
  GIPR antagonist GIP peptide
  PEPTIDE (1)..(37): Peptide
EGTFISEYSIALEKIKQQEFVEWLLAQKPSSGAPPPS

SEQ ID NO: 9 - protein, 37 aa, Artificial Sequence
  GIPR antagonist GIP peptide
  MISC_FEATURE (12)..(12): Xaa = Norleucine
EGTFISEYSIAXEKIKQQEFVEWLLAQKPSSGAPPPS

SEQ ID NO: 10 - protein, 37 aa, Artificial Sequence
  GIPR antagonist GIP peptide
  MISC_FEATURE (11)..(11): Xaa = 2-Aminoisobutyric acid
EGTFISEYSIXLEKIKQQDFVEWLLAQKPSSGAPPPS

SEQ ID NO: 11 - protein, 36 aa, Artificial Sequence
  GIPR antagonist GIP peptide
  MISC_FEATURE (1)..(1): Glutaric acid is attached to the N-terminus of this residue
  MISC_FEATURE (10)..(10): Xaa = 2-Aminoisobutyric acid
GTFISEYSIXLEKIKQQEFVEWLLAQKPSSGAPPPS

SEQ ID NO: 12 - protein, 36 aa, Artificial Sequence
  GIPR antagonist GIP peptide
  MISC_FEATURE (1)..(1): Glutaric acid is attached to the N-terminus of this residue
  MISC_FEATURE (10)..(10): Xaa = 2-Aminoisobutyric acid
  MISC_FEATURE (11)..(11): Xaa = Norleucine
GTFISEYSIXXEKIKQQEFVEWLLAQKPSSGAPPPS

SEQ ID NO: 13 - protein, 36 aa, Artificial Sequence
  GIPR antagonist GIP peptide
  MISC_FEATURE (1)..(1): Glutaric acid is attached to the N-terminus of this residue
GTFISEYSIALEKIKQQEFVEWLLAQKPSSGAPPPS

SEQ ID NO: 14 - protein, 36 aa, Artificial Sequence
  GIPR antagonist GIP peptide
  MISC_FEATURE (1)..(1): Glutaric acid is attached to the N-terminus of this residue
  MISC_FEATURE (10)..(10): Xaa = 2-Aminoisobutyric acid
GTFISEYSIXMEKIKQQEFVEWLLAQKPSSGAPPPS

SEQ ID NO: 15 - protein, 37 aa, Artificial Sequence
  GIPR antagonist GIP peptide
  MISC_FEATURE (11)..(11): Xaa = 2-Aminoisobutyric acid
EGTFISEYSIXMEKIKQQEFVEWLLAQKPSSGAPPPS

SEQ ID NO: 16 - protein, 37 aa, Artificial Sequence
  GIPR antagonist GIP peptide
  PEPTIDE (1)..(37): Peptide
EGTFISEYSIAMEKIKQQEFVEWLLAQKPSSGAPPPS

SEQ ID NO: 17 - protein, 37 aa, Artificial Sequence
  GIPR antagonist GIP peptide
  MISC_FEATURE (11)..(11): Xaa = 2-Aminoisobutyric acid
EGTFISEYSIXLDKIKQQDFVEWLLAQKPSSGAPPPS

SEQ ID NO: 18 - protein, 36 aa, Artificial Sequence
  GIPR antagonist GIP peptide
  MISC_FEATURE (1)..(1): Glutaric acid is attached to the N-terminus of this residue
  MISC_FEATURE (10)..(10): Xaa = 2-Aminoisobutyric acid
GTFISDYSIXMDKIKQQDFVEWLLAQKPSSGAPPPS

SEQ ID NO: 19 - protein, 31 aa, Artificial Sequence
  GLP-1 peptide
  MISC_FEATURE (2)..(2): Xaa = 2-Aminoisobutyric acid
HXEGTFTSDVSSYLEGQAAKEFIAWLVRGRG

SEQ ID NO: 20 - protein, 36 aa, Artificial Sequence; identical to SEQ ID NO: 11
  GIPR antagonist GIP peptide
  MISC_FEATURE (1)..(1): Succinic acid is attached to the N-terminus of this residue
  MISC_FEATURE (10)..(10): Xaa = 2-Aminoisobutyric acid

SEQ ID NO: 21 - protein, 36 aa, Artificial Sequence; identical to SEQ ID NO: 11
  GIPR antagonist GIP peptide
  MISC_FEATURE (1)..(1): Adipic acid is attached to the N-terminus of this residue
  MISC_FEATURE (10)..(10): Xaa = 2-Aminoisobutyric acid

SEQ ID NO: 22 - protein, 39 aa, Artificial Sequence
  GLP-1 peptide
HGEGTFTSDLSKQMEEEAVRLFIEWLKNGGPSSGAPPPS

SEQ ID NO: 23 - protein, 44 aa, Artificial Sequence
  GLP-1 peptide
HGEGTFTSDLSKQMEEEAVRLFIEWLKNGGPSSGAPPSKKKKKK

SEQ ID NO: 24 - protein, 31 aa, Artificial Sequence
  GLP-1 peptide
  MISC_FEATURE (5)..(5): Xaa = Allo-threonine
  MISC_FEATURE (7)..(7): Xaa = Allo-threonine
  MISC_FEATURE (23)..(23): Xaa = Allo-isoleucine
HGEGXFXSDVSSYLEGQAAKEFXDAWLVKGR

SEQ ID NO: 25 - protein, 31 aa, Artificial Sequence
  GLP-1 peptide
HAEGTFTSDVSSYLEGQAAKEFIAWLVKGRG

SEQ ID NO: 26 - protein, 30 aa, Artificial Sequence
  GLP-1 Peptide
  MISC_FEATURE (2)..(2): Xaa = 2-Aminoisobutyric acid
  MISC_FEATURE (29)..(29): Xaa = 2-Aminoisobutyric acid
HXEGTFTSDVSSYLEGQAAKEFIAWLVKXR

SEQ ID NO: 27 - protein, 275 aa, Artificial Sequence
  Peptide
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 28 - protein, 30 aa, Artificial Sequence
  GLP-1 peptide
HAEATFTSDVSSYLEGQAAKEFIAWLVKGR

SEQ ID NO: 29 - protein, 30 aa, Artificial Sequence
  GLP-1 peptide
HGEGTFTSDLSKQMEEEAVRLFIEWLKNGG

SEQ ID NO: 30 - protein, 31 aa, Artificial Sequence
  GLP-1 peptide
DLSKQMEEEAVRLFIEWLKNGGPSSGAPPPS

SEQ ID NO: 31 - protein, 22 aa, Artificial Sequence
  GLP-1 peptide
DLSKQMEEEAVRLFIEWLKNGG

SEQ ID NO: 32 - protein, 37 aa, Artificial Sequence
  GLP-1 peptide
HDEFERHAEGTFTSDVSSYLEGQAAKEFIAWLVKGRG

SEQ ID NO: 33 - protein, 30 aa, Artificial Sequence
  GLP-1 peptide
HAEGTFTSDVSSYLEGQAAKEFIAWLVKGR

SEQ ID NO: 34 - protein, 31 aa, Artificial Sequence; identical to SEQ ID NO: 25
  GLP-1 peptide

SEQ ID NO: 35 - protein, 36 aa, Artificial Sequence
  GLP-1 peptide
HDEFERHAEGTFTSDVSSYLEGQAAKEFIAWLVKGR

SEQ ID NO: 36 - protein, 28 aa, Artificial Sequence
  GLP-1 peptide
EGTFTSDVSSYLEGQAAKEFIAWLVKGR

SEQ ID NO: 37 - protein, 30 aa, Artificial Sequence
  GLP-1 peptide
AAEGTFTSDVSSYLEGQAAKEFIAWLVKGR

SEQ ID NO: 38 - protein, 37 aa, Artificial Sequence
  GIPR antagonist GIP peptide
EGTFISEYSIAMEKIKQQDFVEWLLAQKPSSGAPPPS

(19) United States
(12) Patent Application Publication
(10) Pub. No.: US 2024/0277813 A1
(43) Pub. Date: Aug. 22, 2024

(54) TREATMENT OF OBESITY AND OBESITY-RELATED DISORDERS

(71) Applicant: ANTAG THERAPEUTICS APS, Copenhagen N (DK)

(72) Inventors: Alexander HOVARD SPARRE-ULRICH, Copenhagen (DK); Mette Marie ROSENKILDE, Hellerup (DK)

(21) Appl. No.: 18/565,984

(22) PCT Filed: Jun. 10, 2022
(86) PCT No.: PCT/EP2022/065826

§ 371 Date: Nov. 30, 2023

(30) Foreign Application Priority Data
Jun. 10, 2021 (EP) 21178712.2

(51) Int. Cl. A61K 38/22; A61K 9/00; A61K 38/26; A61P 3/04; A61P 3/06; A61P 5/50; C07K 14/645

(52) U.S. Cl. CPC A61K 38/2235; A61K 9/0019; A61K 38/26; A61P 3/04; A61P 3/06; A61P 5/50; C07K 14/645

(57) ABSTRACT

The present invention relates to a glucose-dependent insulinotropic peptide (GIP) receptor (GIPR) antagonist GIP peptide in combination with a glucagon-like peptide-1 (GLP-1) receptor agonist, such as a GLP-1 peptide, for use in the treatment of obesity and obesity-related disorders, as well as GIPR antagonist GIP peptides for treating a subset of obesity-related disorders.

### TECHNICAL FIELD

The present disclosure relates to a glucose-dependent insulinotropic peptide (GIP) receptor (GIPR) antagonist GIP peptide in combination with a glucagon-like peptide-1 (GLP-1) agonist, such as a GLP-1 peptide, for use in the treatment of obesity and obesity-related disorders, as well as GIPR antagonist GIP peptides for treating a subset of obesity-related disorders.

### BACKGROUND

Gastrointestinal peptides and adipokines are critical signalling molecules involved in controlling whole-body energy homeostasis. These circulating hormones regulate a variety of biological responses such as hunger, satiety and glucose uptake. In vivo experiments have established that these hormones also regulate bone metabolism, while associations between these hormones and bone mass have been observed in human clinical studies.

Incretins are gastrointestinal hormones that help to regulate carbohydrate metabolism in response to food intake. The two main incretins are glucose-dependent insulinotropic peptide (GIP) and glucagon-like peptide-1 (GLP-1), both secreted by intestinal epithelial cells. Intestinal glucagon-like peptide-2 (GLP-2) is co-secreted along with GLP-1 upon nutrient ingestion.

Glucose-dependent insulinotropic peptide (GIP) is a hormone secreted from the K cells of the gut following a meal¹. Like its sister hormone glucagon-like peptide 1 (GLP-1), GIP is a potent insulin secretagogue². In contrast to the glucagonostatic effect of GLP-1^{3, 4}, GIP has been shown to display glucagon-releasing properties under certain conditions (^{3,5-13}). The interest in understanding the biology of GIP was intensified by the association between rodent GIPR (GIP receptor) and adiposity¹⁴⁻²¹. In humans, although less clear, there is likewise evidence for a role of GIP in fat metabolism with the demonstration of the GIPR expression in adipose tissue²², an association between high BMI and increased GIP levels^{22, 23}, increased adipose tissue blood flow and TAG (triacylglycerol) deposition following GIP administration in a state of high insulin and high glucose²⁴, decreased basal and postprandial GIP levels observed in obese children put on a diet²⁵, and increased fasting GIP levels observed in healthy young men put on a high fat diet²⁶. Thus, the potential as an anti-obesity agent has attracted additional attention for the development of potent GIPR antagonists.

Examples of potent GIPR antagonists GIP peptides are disclosed in PCT/EP2020/084487.

GLP-1 is a 31-amino acid peptide derived from the proglucagon gene. It is secreted by intestinal L-cells and released in response to food ingestion to induce insulin secretion from pancreatic β-cells. In addition to the incretin effects, GLP-1 also decreases glucagon secretion, delays gastric emptying and reduces caloric intake. GLP-1 exerts its effects by activation of the GLP-1 receptor, which belongs to a class B G-protein-coupled receptor. The function of GLP-1 is limited by rapid degradation by the DPP-IV enzyme, resulting in a half-life of approximately 2 minutes.

Recently, long-lasting GLP-1 receptor agonists such as exenatide, liraglutide, dulaglutide and semaglutide have been developed and are used in the clinic to improve glycemic control in patients with type 2 diabetes. Furthermore, GLP-1 receptor agonists promote body weight reduction as well as reduction in blood pressure and plasma cholesterol levels in patients.

Irwin et al. 2009 (Diabetes Obes Metab 2009 June; 11(6):603-10) discloses that daily intraperitoneal injections of the GLP-1 agonist (d-Ala8)GLP-1 or of the GIPR antagonist GIP peptide (Pro3)GIP restored glycaemic control to normal levels and significantly improved glucose tolerance compared with high-fat controls in mice. However, food intake and body weights were not affected. Moreover, Irwin et al. 2009 also discloses that combination therapy consisting of the GLP-1 agonist (d-Ala8)GLP-1 and the GIPR antagonist GIP peptide (Pro3)GIP seems to have little benefit over either treatment alone, and that body weight and food intake were not affected even when the two compounds were administered together.

West et al. 2021 (PLOS One 2021 Mar. 31; 16(3)) discloses administration of a GIPR antagonist GIP peptide, GIPA-1: mouse GIP(3-30)NH₂, or GIPA-2 (NaAc-K10[γEγE-C16]-Arg18-hGIP(5-42), alone or in combination with the GLP-1 agonist Liraglutide, to mice on a fat-diet, or to lean mice. GIPA-2 was found to be a more potent antagonist than GIPA-1 in mice, and inhibited glucose stimulated insulin secretion. However, chronic administration of GIPA-1 or GIPA-2 alone had no effect on absolute body weight or cumulative food intake compared with vehicle control. As expected, liraglutide reduced both body weight and food intake, but no additive effect was observed by combining liraglutide with either of the GIPR antagonists on these parameters.

### SUMMARY

The present inventors have found that acylated GIPR antagonist GIP peptides derived from native hGIP(3-30) comprising amino acid substitutions D9E, A13Aib, D15E, H18K, D21E and/or N24E and a C-terminal elongation, which have high solubility, physical stability and/or superior antagonistic properties, are surprisingly effective in reducing body weight, reducing food intake, reducing fasting blood levels of glucose, reducing fasting blood levels of insulin, reducing insulin resistance and improving insulin sensitivity, reducing fasting blood levels of triglycerides and reducing fasting blood levels of cholesterol, in a monkey model of obesity, alone and in particular when used in combination with the GLP-1 receptor agonist liraglutide. This implies that these carefully optimised GIPR antagonist GIP peptides are especially effective especially when administered in combination with GLP-1 receptor agonists in treating obesity and obesity-related disorders such as dyslipidemia.

The in vivo data presented herein demonstrate for the first time that the herein disclosed therapy such as combination therapy results in improved reduction of body weight, improved reduction of food intake, improved reduction of fasting blood levels of glucose, improved reduction of fasting blood levels of insulin, improved reduction of insulin resistance, improved reduction of fasting blood levels of triglycerides, improved reduction of fasting blood levels of cholesterol and improved reduction of fasting blood levels of low-density lipoprotein (LDL) cholesterol compared to any of the single therapies, and there is at least a clear additive effect on these parameters.

Thus, in one aspect the present disclosure provides a glucose-dependent insulinotropic peptide receptor (GIPR) antagonist GIP peptide consisting of amino acid sequence SEQ ID NO:1:

(SEQ ID NO: 1)
3 -4 - 5 - 6 7 8 9 10 11 12 13 14 15 16
X₁-G - T - F - I - S - E - Y - S - I - Aib - X₂ - E - K-
17 18 19 20 21 22 23 24 25 26 27 28 29 30 31
I - K - Q - Q - E - F - V - E - W - L - L - A - Q - K - P
32 33 34 35 36 37 38 39
S - S - G - A - P - P - P - S,

wherein X₁ is E, glutaric acid, adipic acid or succinic acid;
wherein X₂ is M, L or Nle;
or a functional variant thereof, wherein the amino acid sequence of said variant differs from SEQ ID NO:1 in that the amino acid sequence of the variant comprises 1, 2 or 3 individual amino acid substitutions,
wherein said peptide comprises a fatty acid molecule attached to the side chain amino group of the amino acid residue at position 18 of SEQ ID NO:1, or said functional variant thereof,
for use in a method of treating obesity, obesity-related disorders and/or dyslipidemia, said method comprising one or more steps of co-administering a glucagon-like peptide-1 (GLP-1) receptor agonist.

In another aspect, the present disclosure provides a GIPR antagonist GIP peptide as disclosed herein, for use in a method of inhibiting or reducing one or more of i) food intake, ii) body weight, iii) fasting blood levels of glucose, iv) fasting blood levels of insulin, v) insulin resistance, vi) fasting blood levels of triglycerides, vii) fasting blood levels of cholesterol, viii) fasting blood levels of low-density lipoprotein (LDL) cholesterol, said method comprising one or more steps of co-administering a glucagon-like peptide-1 (GLP-1) receptor agonist.

In another aspect, the present disclosure provides a GIPR antagonist GIP peptide as disclosed herein, for use in a method of treating dyslipidemia, said method comprising one or more steps of co-administering a glucagon-like peptide-1 (GLP-1) receptor agonist.

A further aspect of the present disclosure provides a composition comprising, separately or together, a GIPR antagonist GIP peptide and a GLP-1 receptor agonist, wherein said GIPR antagonist GIP peptide consist of amino acid sequence SEQ ID NO:1:

(SEQ ID NO: 1)
3 -4 - 5 - 6 7 8 9 10 11 12 13 14 15 16
X₁-G - T - F - I - S - E - Y - S - I - Aib - X₂ - E - K-
17 18 19 20 21 22 23 24 25 26 27 28 29 30 31
I - K - Q - Q - E - F - V - E - W - L - L - A - Q - K - P
32 33 34 35 36 37 38 39
S - S - G - A - P - P - P - S,

wherein X₁ is E, glutaric acid, adipic acid or succinic acid;
wherein X₂ is M, L or Nle;
or a functional variant thereof, wherein the amino acid sequence of said variant differs from SEQ ID NO:1 in that the amino acid sequence of the variant comprises 1, 2 or 3 individual amino acid substitutions,
wherein said peptide comprises a fatty acid molecule attached to the side chain amino group of the amino acid residue at position 18 of SEQ ID NO:1, or said functional variant thereof.

A further aspect of the present disclosure provides a kit of parts comprising a GIPR antagonist GIP peptide as defined herein and a GLP-1 receptor agonist as defined herein.

In one embodiment said composition comprising, separately or together, or said kit of parts comprising a GIPR antagonist GIP peptide and a GLP-1 receptor agonist, is for use in a method of treating one or more of obesity, obesity-related disorders and dyslipidemia; and/or for use in a method of inhibiting or reducing one or more of i) food intake, ii) body weight, iii) fasting blood levels of glucose, iv) fasting blood levels of insulin, v) insulin resistance, vi) fasting blood levels of triglycerides, vii) fasting blood levels of cholesterol, and viii) fasting blood levels of low-density lipoprotein (LDL) cholesterol.

Moreover, the present inventors have found that the optimised GIPR antagonist GIP peptides of the present disclosure are particularly effective in improving a number of parameters inclusive of reducing fasting blood levels of insulin, reducing insulin resistance and improving insulin sensitivity, reducing fasting blood levels of triglycerides and reducing fasting blood levels of low-density lipoprotein (LDL) cholesterol, which occur independently of the observed effects on weight loss and obesity. This demonstrates that the optimised GIPR antagonist GIP peptides of the present disclosure have a dual effect on insulin sensitivity and blood lipids; directly as well as indirectly via a reduction of food intake and body weight.

Thus, one aspect of the present disclosure relates to a GIPR antagonist GIP peptide consisting of amino acid sequence SEQ ID NO:1:

(SEQ ID NO: 1)
3 -4 - 5 - 6 7 8 9 10 11 12 13 14 15 16
X₁-G - T - F - I - S - E - Y - S - I - Aib - X₂ - E - K-
17 18 19 20 21 22 23 24 25 26 27 28 29 30 31
I - K - Q - Q - E - F - V - E - W - L - L - A - Q - K - P
32 33 34 35 36 37 38 39
S - S - G - A - P - P - P - S,

wherein X₁ is E, glutaric acid, adipic acid or succinic acid;
wherein X₂ is M, L or Nle;
or a functional variant thereof, wherein the amino acid sequence of said variant differs from SEQ ID NO:1 in that the amino acid sequence of the variant comprises 1, 2 or 3 individual amino acid substitutions,
wherein said peptide comprises a fatty acid molecule attached to the side chain amino group of the amino acid residue at position 18 of SEQ ID NO:1, or said functional variant thereof,
for use in a method of inhibiting or reducing one or more of i) food intake, ii) body weight, iii) fasting blood levels of glucose, iv) fasting blood levels of insulin, v) insulin resistance, vi) fasting blood levels of triglycerides, vii) fasting blood levels of cholesterol, and viii) fasting blood levels of low-density lipoprotein (LDL) cholesterol.

Another aspect of the present disclosure relates to a GIPR antagonist GIP peptide consisting of amino acid sequence SEQ ID NO:1:

(SEQ ID NO: 1)
3 -4 - 5 - 6 7 8 9 10 11 12 13 14 15 16
X₁-G - T - F - I - S - E - Y - S - I - Aib - X₂ - E - K-
17 18 19 20 21 22 23 24 25 26 27 28 29 30 31
I - K - Q - Q - E - F - V - E - W - L - L - A - Q - K - P
32 33 34 35 36 37 38 39
S - S - G - A - P - P - P - S,

wherein X₁ is E, glutaric acid, adipic acid or succinic acid;
wherein X₂ is M, L or Nle;
or a functional variant thereof, wherein the amino acid sequence of said variant differs from SEQ ID NO:1 in that the amino acid sequence of the variant comprises 1, 2 or 3 individual amino acid substitutions,
wherein said peptide comprises a fatty acid molecule attached to the side chain amino group of the amino acid residue at position 18 of SEQ ID NO:1, or said functional variant thereof,
for use in a method of treating obesity, obesity-related disorders and/or dyslipidemia.

### DESCRIPTION OF DRAWINGS

FIG. 1: GIPR antagonists alone and in combination with the GLP-1R agonist liraglutide markedly reduce energy intake and body weight in HFD induced obese male Cynomolgus monkeys, and improve insulin resistance and blood lipid profiles. Cynomolgus monkeys (Macaca fascicularis) were fed a HFD consisting of 50 g of KBI proprietary standard monkey formula feed in the morning 9:00 to 10:00 AM, 150 g apple in the afternoon 14:00 to 15:00 PM, and 100 g of KBI proprietary HFD in the evening 16:00 to 17:00 PM. Water was provided ad libitum. The monkeys were treated for 42 days with vehicle (placebo), liraglutide (in a dose escalation regimen over 7 days up to 0.03 mg/kg, once daily SC injection), Compound 1 (540 nmol/kg, once daily SC injection), Compound 1 in combination with liraglutide (same dosing and frequency), Compound 2 (1440 nmol/kg the first 3 days and 540 nmol/kg the remaining 39 study days, once daily SC injection), or Compound 2 in combination with liraglutide (same dosing and frequency). A) Cumulative energy intake. B) Percent change in body weight. C) Homeostatic Model Assessment for Insulin Resistance. D) Fasting Triglycerides. E) Fasting low-density lipoprotein cholesterol. F) Changes in fasting low-density lipoprotein cholesterol from baseline. Values represent means±SEM, n=9-10 per group.

### DEFINITIONS

The term “agonist” in the present context refers to a peptide, or analogue thereof, capable of binding to and activating downstream signalling cascades from a receptor.

The term “antagonist” in the present context refers to a GIPR antagonist GIP peptide as defined herein, capable of binding to and blocking or reducing agonist-mediated responses of a receptor. Antagonists usually do not provoke a biological response themselves upon binding to a receptor. Antagonists have affinity but no efficacy for their cognate receptors, and binding of an antagonist to its receptor will inhibit the function of an agonist or inverse agonist at receptors. Antagonists mediate their effects by binding to the active (orthosteric) site or to allosteric sites on receptors, or they may interact at unique binding sites not normally involved in the biological regulation of the receptor's activity. Antagonist activity may be reversible or irreversible depending on the longevity of the antagonist-receptor complex, which, in turn, depends on the nature of antagonist-receptor binding. The majority of drug antagonists typically achieve their potency by competing with endogenous ligands or substrates at structurally defined binding sites on receptors. Antagonists may be competitive, non-competitive, uncompetitive, silent antagonists, partial agonists or inverse agonists.

The term “glucose-dependent insulinotropic polypeptide (GIP) receptor (GIPR) antagonists” as used herein refers to a compound, such as a peptide, capable of binding to and blocking or reducing agonist-mediated responses of GIPR.

The term “GLP-1 receptor agonist” as used herein refers to a compound, such as a peptide or a small molecule drug, capable of binding to and activating or enhancing agonist-mediated responses of GLP-1R.

The term “individual” refers to vertebrates, particular members of the mammalian species, preferably primates including humans. As used herein, ‘subject’ and ‘individual’ may be used interchangeably.

An “amino acid residue” can be a natural or non-natural amino acid residue linked by peptide bonds or bonds different from peptide bonds. The amino acid residues can be in D-configuration or L-configuration. An amino acid residue comprises an amino terminal part (NH₂) and a carboxy terminal part (COOH) separated by a central part comprising a carbon atom, or a chain of carbon atoms, at least one of which comprises at least one side chain or functional group. NH₂ refers to the amino group present at the amino terminal end of an amino acid or peptide, and COOH refers to the carboxy group present at the carboxy terminal end of an amino acid or peptide. The generic term amino acid comprises both natural and non-natural amino acids. Natural amino acids of standard nomenclature as listed in J. Biol. Chem., 243:3552-59 (1969) and adopted in 37 C.F.R., section 1.822(b)(2) belong to the group of amino acids listed herewith: Y,G,F,M,A,S,I,L,T,V,P,K,H,Q,E,W,R,D,N and C. Non-natural amino acids are those not listed immediately above. Also, non-natural amino acid residues include, but are not limited to, modified amino acid residues, L-amino acid residues, and stereoisomers of D-amino acid residues.

An “equivalent amino acid residue” refers to an amino acid residue capable of replacing another amino acid residue in a polypeptide without substantially altering the structure and/or functionality of the polypeptide. Equivalent amino acids thus have similar properties such as bulkiness of the side-chain, side chain polarity (polar or non-polar), hydrophobicity (hydrophobic or hydrophilic), pH (acidic, neutral or basic) and side chain organization of carbon molecules (aromatic/aliphatic). As such, “equivalent amino acid residues” can be regarded as “conservative amino acid substitutions”, and it is the substitution of amino acids whose side chains have similar biochemical properties and thus do not affect the function of the peptide.

Among the common amino acids, for example, a “conservative amino acid substitution” can also be illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine.

Within the meaning of the term “equivalent amino acid substitution” as applied herein, one amino acid may be substituted for another, in one embodiment, within the groups of amino acids indicated herein below:
i) Amino acids having polar side chains (Asp, Glu, Lys, Arg, His, Asn, Gln, Ser, Thr, Tyr, and Cys,)
ii) Amino acids having non-polar side chains (Gly, Ala, Val, Leu, lie, Phe, Trp, Pro, and Met)
iii) Amino acids having aliphatic side chains (Gly, Ala Val, Leu, lie)
iv) Amino acids having cyclic side chains (Phe, Tyr, Trp, His, Pro)
v) Amino acids having aromatic side chains (Phe, Tyr, Trp)
vi) Amino acids having acidic side chains (Asp, Glu)
vii) Amino acids having basic side chains (Lys, Arg, His)
viii) Amino acids having amide side chains (Asn, Gln)
ix) Amino acids having hydroxy side chains (Ser, Thr, Tyr)
x) Amino acids having sulphur-containing side chains (Cys, Met),
xi) Neutral, weakly hydrophobic amino acids (Pro, Ala, Gly, Ser, Thr)
xii) Hydrophilic, acidic amino acids (Gln, Asn, Glu, Asp), and
xiii) Hydrophobic amino acids (Leu, lie, Val)

In addition, a serine residue of a peptide of the present disclosure may be substituted with an amino acid selected from the group consisting of Gln, Asn and Thr (all amino acids with polar uncharged side chains); and independently thereof, a glycine residue (Gly) is substituted with an amino acid selected from the group consisting of Ala, Val, Leu, and lie; and independently thereof, an arginine residue (Arg) is substituted with an amino acid selected from the group consisting of Lys and His (all have positively charged side chains); and independently thereof, a lysine residue (Lys) may be substituted with an amino acid selected from the group consisting of Arg and His; and independently thereof, a methionine residue (Met) may be substituted with an amino acid selected from the group consisting of Leu, Pro, lie, Val, Phe, Tyr and Trp (all have hydrophobic side chains); and independently thereof, a glutamine residue (Gln) may be substituted with an amino acid selected from the group consisting of Asp, Glu, and Asn; and independently thereof, an alanine residue (Ala) may be substituted with an amino acid selected from the group consisting of Gly, Val, Leu, and lie.

Where the L or D form (optical isomers) has not been specified it is to be understood that the amino acid in question has the natural L form, cf. Pure & Appl. Chem. Vol. (56(5) pp 595-624 (1984) or the D form, so that the peptides formed may be constituted of amino acids of L form, D form, or a sequence of mixed L forms and D forms.

As used herein, a Glutamic acid (Glu) mimetic is a moiety, with two carboxy functional groups separated by three carbon atoms. Examples are beta-Glu, gamma-Glu or glutaric acid. Glutaric acid is also known as Pentanedioic acid.

A “functional variant” of a peptide is a peptide capable of performing essentially the same functions as the peptide it is a functional variant of. In particular, a functional variant can essentially bind the same molecules, such as receptors, or perform the same receptor mediated responses as the peptide it is a functional variant of. A functional variant of a “glucose-dependent insulinotropic peptide (GIP) antagonist GIP peptide” is a peptide, which can bind to the GIPR and inhibit GIPR downstream signalling, such as cAMP generation.

A “bioactive agent” (i.e. a biologically active substance/agent) is any agent, drug, compound, composition of matter or mixture which provides some pharmacologic, often beneficial, effect that can be demonstrated in vivo or in vitro. It refers to the GIPR antagonist GIP peptide as defined herein and compounds or compositions comprising these, e.g. a composition comprising the GIPR antagonist GIP peptide as defined herein and the GLP-1 receptor agonist as defined herein, only the GIPR antagonist GIP peptide as defined herein, or only the GLP-1 receptor agonist as defined herein. As used herein, this term further includes any physiologically or pharmacologically active substance that produces a localized or systemic effect in an individual.

The terms “drug” and “medicament” as used herein include biologically, physiologically, or pharmacologically active substances that act locally or systemically in the human or animal body.

The terms “treatment” and “treating” as used herein refer to the management and care of a patient for the purpose of combating a condition, disease or disorder. The term is intended to include the full spectrum of treatments for a given condition from which the patient is suffering, and refer equally to curative therapy, prophylactic or preventative therapy and ameliorating or palliative therapy, such as administration of the bioactive agents for the purpose of: alleviating or relieving symptoms or complications; delaying the progression of the condition, partially arresting the clinical manifestations, disease or disorder; curing or eliminating the condition, disease or disorder; amelioration or palliation of the condition or symptoms, and remission (whether partial or total), whether detectable or undetectable; and/or preventing or reducing the risk of acquiring the condition, disease or disorder, wherein “preventing” or “prevention” is to be understood to refer to the management and care of a patient for the purpose of hindering the development of the condition, disease or disorder, and includes the administration of the bioactive agents to prevent or reduce the risk of the onset of symptoms or complications. The term “palliation”, and variations thereof, as used herein, means that the extent and/or undesirable manifestations of a physiological condition or symptom are lessened and/or time course of the progression is slowed or lengthened, as compared to not administering bioactive agents of the present invention.

The individual to be treated is preferably a mammal, in particular a human being. Treatment of animals, such as mice, rats, dogs, cats, cows, horses, sheep and pigs, is, however, also encompassed herewith.

An “individual in need thereof” refers to an individual who may benefit from the present disclosure. In one embodiment, said individual in need thereof is an obese or overweight individual.

A treatment according to the invention can be prophylactic, ameliorating and/or curative.

“Pharmacologically effective amount”, “pharmaceutically effective amount” or “physiologically effective amount” of a bioactive agent is the amount of a bioactive agent present in a pharmaceutical composition as described herein that is needed to provide a desired level of active agent in the bloodstream or at the site of action in an individual (e.g. the lungs, the gastric system, the colorectal system, prostate, etc.) to be treated to give an anticipated physiological response when such composition is administered. A bioactive agent in the present context refers to a GIPR antagonist GIP peptide as disclosed herein.

“Co-administering” or “co-administration” as used herein refers to the administration of a GIPR antagonist GIP peptide of the present disclosure and a state-of-the-art pharmaceutical composition comprising a GLP-1 receptor agonist. The at least two components can be administered separately, sequentially or simultaneously.

### DETAILED DESCRIPTION

### Combination Therapy

In one aspect the present disclosure provides a glucose-dependent insulinotropic peptide receptor (GIPR) antagonist GIP peptide consisting of amino acid sequence SEQ ID NO:1:

(SEQ ID NO: 1)
3 -4 - 5 - 6 7 8 9 10 11 12 13 14 15 16
X₁-G - T - F - I - S - E - Y - S - I - Aib - X₂ - E - K-
17 18 19 20 21 22 23 24 25 26 27 28 29 30 31
I - K - Q - Q - E - F - V - E - W - L - L - A - Q - K - P
32 33 34 35 36 37 38 39
S - S - G - A - P - P - P - S,

wherein X₁ is E, glutaric acid, adipic acid or succinic acid;
wherein X₂ is M, L or Nle;
or a functional variant thereof, wherein the amino acid sequence of said variant differs from SEQ ID NO:1 in that the amino acid sequence of the variant comprises 1, 2 or 3 individual amino acid substitutions,
wherein said peptide comprises a fatty acid molecule attached to the side chain amino group of the amino acid residue at position 18 of SEQ ID NO:1, or said functional variant thereof,
for use in a method of treating obesity, obesity-related disorders and/or dyslipidemia, said method comprising one or more steps of co-administering a glucagon-like peptide-1 (GLP-1) receptor agonist.

Also disclosed is a GIPR antagonist GIP peptide consisting of amino acid sequence SEQ ID NO:1

(SEQ ID NO: 1)
3 -4 - 5 - 6 7 8 9 10 11 12 13 14 15 16
X₁-G - T - F - I - S - E - Y - S - I - Aib - X₂ - E - K-
17 18 19 20 21 22 23 24 25 26 27 28 29 30 31
I - K - Q - Q - E - F - V - E - W - L - L - A - Q - K - P
32 33 34 35 36 37 38 39
S - S - G - A - P - P - P - S,

wherein X₁ is E, glutaric acid, adipic acid or succinic acid;
wherein X₂ is M, L or Nle;
or a functional variant thereof, wherein the amino acid sequence of said variant differs from SEQ ID NO:1 in that the amino acid sequence of the variant comprises 1, 2 or 3 individual amino acid substitutions,
wherein said peptide comprises a fatty acid molecule attached to the side chain amino group of the amino acid residue at position 18 of SEQ ID NO:1, or said functional variant thereof,
for use in a method of inhibiting or reducing one or more of i) food intake, ii) body weight, iii) fasting blood levels of glucose, iv) fasting blood levels of insulin, v) insulin resistance, vi) fasting blood levels of triglycerides, vii) fasting blood levels of cholesterol, and viii) fasting blood levels of low-density lipoprotein (LDL) cholesterol, said method comprising one or more steps of co-administering a glucagon-like peptide-1 (GLP-1) receptor agonist.

Also disclosed is a GIPR antagonist GIP peptide consisting of amino acid sequence SEQ ID NO:1:

(SEQ ID NO: 1)
3 -4 - 5 - 6 7 8 9 10 11 12 13 14 15 16
X₁-G - T - F - I - S - E - Y - S - I - Aib - X₂ - E - K-
17 18 19 20 21 22 23 24 25 26 27 28 29 30 31
I - K - Q - Q - E - F - V - E - W - L - L - A - Q - K - P
32 33 34 35 36 37 38 39
S - S - G - A - P - P - P - S,

wherein X₁ is E, glutaric acid, adipic acid or succinic acid;
wherein X₂ is M, L or Nle;
or a functional variant thereof, wherein the amino acid sequence of said variant differs from SEQ ID NO:1 in that the amino acid sequence of the variant comprises 1, 2 or 3 individual amino acid substitutions,
wherein said peptide comprises a fatty acid molecule attached to the side chain amino group of the amino acid residue at position 18 of SEQ ID NO:1, or said functional variant thereof,
for use in a method of treating dyslipidemia, said method comprising one or more steps of co-administering a glucagon-like peptide-1 (GLP-1) receptor agonist.

Also disclosed is the use of a composition comprising a GLP-1R agonist and a GIPR antagonist GIP peptide consisting of amino acid sequence SEQ ID NO:1:

(SEQ ID NO: 1)
3 -4 - 5 - 6 7 8 9 10 11 12 13 14 15 16
X₁-G - T - F - I - S - E - Y - S - I - Aib - X₂ - E - K-
17 18 19 20 21 22 23 24 25 26 27 28 29 30 31
I - K - Q - Q - E - F - V - E - W - L - L - A - Q - K - P
32 33 34 35 36 37 38 39
S - S - G - A - P - P - P - S,

wherein X₁ is E, glutaric acid, adipic acid or succinic acid;

wherein X₂ is M, L or Nle;

or a functional variant thereof, wherein the amino acid sequence of said variant differs from SEQ ID NO:1 in that the amino acid sequence of the variant comprises 1, 2 or 3 individual amino acid substitutions,

wherein said peptide comprises a fatty acid molecule attached to the side chain amino group of the amino acid residue at position 18 of SEQ ID NO:1, or said functional variant thereof,

in the manufacture of a medicament for treatment of obesity, obesity-related disorders and/or dyslipidemia.

Also disclosed is the use of a composition comprising a GLP-1R agonist and a GIPR antagonist GIP peptide consisting of amino acid sequence SEQ ID NO:1:

(SEQ ID NO: 1)
3 -4 - 5 - 6 7 8 9 10 11 12 13 14 15 16
X₁-G - T - F - I - S - E - Y - S - I - Aib - X₂ - E - K-
17 18 19 20 21 22 23 24 25 26 27 28 29 30 31
I - K - Q - Q - E - F - V - E - W - L - L - A - Q - K - P
32 33 34 35 36 37 38 39
S - S - G - A - P - P - P - S,

wherein X₁ is E, glutaric acid, adipic acid or succinic acid;

wherein X₂ is M, L or Nle;

or a functional variant thereof, wherein the amino acid sequence of said variant differs from SEQ ID NO:1 in that the amino acid sequence of the variant comprises 1, 2 or 3 individual amino acid substitutions,

wherein said peptide comprises a fatty acid molecule attached to the side chain amino group of the amino acid residue at position 18 of SEQ ID NO:1, or said functional variant thereof,

in the manufacture of a medicament for inhibiting or reducing one or more of i) food intake, ii) body weight, iii) fasting blood levels of glucose, iv) fasting blood levels of insulin, v) insulin resistance, vi) fasting blood levels of triglycerides, vii) fasting blood levels of cholesterol, viii) fasting blood levels of low-density lipoprotein (LDL) cholesterol.

Also disclosed is the use of a composition comprising a GLP-1R agonist and a GIPR antagonist GIP peptide consisting of amino acid sequence SEQ ID NO:1:

(SEQ ID NO: 1)
3 -4 - 5 - 6 7 8 9 10 11 12 13 14 15 16
X₁-G - T - F - I - S - E - Y - S - I - Aib - X₂ - E - K-
17 18 19 20 21 22 23 24 25 26 27 28 29 30 31
I - K - Q - Q - E - F - V - E - W - L - L - A - Q - K - P
32 33 34 35 36 37 38 39
S - S - G - A - P - P - P - S,

wherein X₁ is E, glutaric acid, adipic acid or succinic acid;

wherein X₂ is M, L or Nle;

or a functional variant thereof, wherein the amino acid sequence of said variant differs from SEQ ID NO:1 in that the amino acid sequence of the variant comprises 1, 2 or 3 individual amino acid substitutions,

wherein said peptide comprises a fatty acid molecule attached to the side chain amino group of the amino acid residue at position 18 of SEQ ID NO:1, or said functional variant thereof,

in the manufacture of a medicament for treating dyslipidemia.

Also disclosed herein is a method for treatment of obesity, obesity-related disorders and/or dyslipidemia comprising administration to an individual in need thereof a GIPR antagonist GIP peptide consisting of amino acid sequence SEQ ID NO:1:

(SEQ ID NO: 1)
3 - 4 - 5 - 6 7 8 9 10 11 12 13 14 15 16
X₁ - G - T - F - I - S - E - Y - S - I - Aib - X₂ - E - K -
17 18 19 20 21 22 23 24 25 26 27 28 29 30 31
I - K - Q - Q - E - F - V - E - W - L - L - A - Q - K - P
32 33 34 35 36 37 38 39
S - S - G - A - P - P - P - S,

wherein X₁ is E, glutaric acid, adipic acid or succinic acid;

wherein X₂ is M, L or Nle;

or a functional variant thereof, wherein the amino acid sequence of said variant differs from SEQ ID NO:1 in that the amino acid sequence of the variant comprises 1, 2 or 3 individual amino acid substitutions,

wherein said peptide comprises a fatty acid molecule attached to the side chain amino group of the amino acid residue at position 18 of SEQ ID NO:1, or said functional variant thereof,

said method comprising one or more steps of co-administering a GLP-1R agonist.

Also disclosed herein is a method for inhibiting or reducing one or more of i) food intake, ii) body weight, iii) fasting blood levels of glucose, iv) fasting blood levels of insulin, v) insulin resistance, vi) fasting blood levels of triglycerides, vii) fasting blood levels of cholesterol and viii) fasting blood levels of low-density lipoprotein (LDL) cholesterol in an individual in need thereof, said method comprising administration of a GIPR antagonist GIP peptide consisting of amino acid sequence SEQ ID NO:1:

(SEQ ID NO: 1)
3 - 4 - 5 - 6 7 8 9 10 11 12 13 14 15 16
X₁ - G - T - F - I - S - E - Y - S - I - Aib - X₂ - E - K -
17 18 19 20 21 22 23 24 25 26 27 28 29 30 31
I - K - Q - Q - E - F - V - E - W - L - L - A - Q - K - P
32 33 34 35 36 37 38 39
S - S - G - A - P - P - P - S,

wherein X₁ is E, glutaric acid, adipic acid or succinic acid;

wherein X₂ is M, L or Nle;

or a functional variant thereof, wherein the amino acid sequence of said variant differs from SEQ ID NO:1 in that the amino acid sequence of the variant comprises 1, 2 or 3 individual amino acid substitutions,

wherein said peptide comprises a fatty acid molecule attached to the side chain amino group of the amino acid residue at position 18 of SEQ ID NO:1, or said functional variant thereof,

said method comprising one or more steps of co-administering a GLP-1R agonist.

In one aspect, the present disclosure provides a composition comprising, separately or together, or a kit of parts comprising, a GIPR antagonist GIP peptide and a GLP-1 receptor agonist,

wherein said GIPR antagonist GIP peptide consist of amino acid sequence SEQ ID NO:1:

(SEQ ID NO: 1)
3 - 4 - 5 - 6 7 8 9 10 11 12 13 14 15 16
X₁ - G - T - F - I - S - E - Y - S - I - Aib - X₂ - E - K -
17 18 19 20 21 22 23 24 25 26 27 28 29 30 31
I - K - Q - Q - E - F - V - E - W - L - L - A - Q - K - P
32 33 34 35 36 37 38 39
S - S - G - A - P - P - P - S,

wherein X₁ is E, glutaric acid, adipic acid or succinic acid;

wherein X₂ is M, L or Nle;

or a functional variant thereof, wherein the amino acid sequence of said variant differs from SEQ ID NO:1 in that the amino acid sequence of the variant comprises 1, 2 or 3 individual amino acid substitutions,

wherein said peptide comprises a fatty acid molecule attached to the side chain amino group of the amino acid residue at position 18 of SEQ ID NO:1, or said functional variant thereof.

Also disclosed herein is provided a composition comprising, separately or together, or a kit of parts comprising, a GIPR antagonist GIP peptide and a GLP-1 receptor agonist, wherein said GIPR antagonist GIP peptide consist of amino acid sequence SEQ ID NO:1:

(SEQ ID NO: 1)
3 - 4 - 5 - 6 7 8 9 10 11 12 13 14 15 16
X₁ - G - T - F - I - S - E - Y - S - I - Aib - X₂ - E - K -
17 18 19 20 21 22 23 24 25 26 27 28 29 30 31
I - K - Q - Q - E - F - V - E - W - L - L - A - Q - K - P
32 33 34 35 36 37 38 39
S - S - G - A - P - P - P - S,

wherein X₁ is E, glutaric acid, adipic acid or succinic acid;

wherein X₂ is M, L or Nle;

or a functional variant thereof, wherein the amino acid sequence of said variant differs from SEQ ID NO:1 in that the amino acid sequence of the variant comprises 1, 2 or 3 individual amino acid substitutions,

wherein said peptide comprises a fatty acid molecule attached to the side chain amino group of the amino acid residue at position 18 of SEQ ID NO:1, or said functional variant thereof,

for use in a method for treatment of obesity, obesity-related disorders and/or dyslipidemia.

Also disclosed herein is a composition comprising, separately or together, or a kit of parts comprising, a GIPR antagonist GIP peptide and a GLP-1 receptor agonist, wherein said GIPR antagonist GIP peptide consist of amino acid sequence SEQ ID NO:1:

(SEQ ID NO: 1)
3 - 4 - 5 - 6 7 8 9 10 11 12 13 14 15 16
X₁ - G - T - F - I - S - E - Y - S - I - Aib - X₂ - E - K -
17 18 19 20 21 22 23 24 25 26 27 28 29 30 31
I - K - Q - Q - E - F - V - E - W - L - L - A - Q - K - P
32 33 34 35 36 37 38 39
S - S - G - A - P - P - P - S,

wherein X₁ is E, glutaric acid, adipic acid or succinic acid;

wherein X₂ is M, L or Nle;

or a functional variant thereof, wherein the amino acid sequence of said variant differs from SEQ ID NO:1 in that the amino acid sequence of the variant comprises 1, 2 or 3 individual amino acid substitutions,

wherein said peptide comprises a fatty acid molecule attached to the side chain amino group of the amino acid residue at position 18 of SEQ ID NO:1, or said functional variant thereof,

for use in a method for inhibiting or reducing one or more of i) food intake, ii) body weight, iii) fasting blood levels of glucose, iv) fasting blood levels of insulin, v) insulin resistance, vi) fasting blood levels of triglycerides, vii) fasting blood levels of cholesterol and viii) fasting blood levels of low-density lipoprotein (LDL) cholesterol.

Also disclosed is a combination drug comprising i) a glucagon-like peptide-1 (GLP-1) receptor agonist, and ii) a glucose-dependent insulinotropic peptide receptor (GIPR) antagonist GIP peptide consisting of amino acid sequence SEQ ID NO:1:

(SEQ ID NO: 1)
3 - 4 - 5 - 6 7 8 9 10 11 12 13 14 15 16
X₁ - G - T - F - I - S - E - Y - S - I - Aib - X₂ - E - K -
17 18 19 20 21 22 23 24 25 26 27 28 29 30 31
I - K - Q - Q - E - F - V - E - W - L - L - A - Q - K - P
32 33 34 35 36 37 38 39
S - S - G - A - P - P - P - S,

wherein X₁ is E, glutaric acid, adipic acid or succinic acid;

wherein X₂ is M, L or Nle;

or a functional variant thereof, wherein the amino acid sequence of said variant differs from SEQ ID NO:1 in that the amino acid sequence of the variant comprises 1, 2 or 3 individual amino acid substitutions,

wherein said peptide comprises a fatty acid molecule attached to the side chain amino group of the amino acid residue at position 18 of SEQ ID NO:1, or said functional variant thereof.

Also disclosed is a combination drug comprising i) a glucagon-like peptide-1 (GLP-1) receptor agonist, and ii) a glucose-dependent insulinotropic peptide receptor (GIPR) antagonist GIP peptide consisting of amino acid sequence SEQ ID NO:1:

(SEQ ID NO: 1)
3 - 4 - 5 - 6 7 8 9 10 11 12 13 14 15 16
X₁ - G - T - F - I - S - E - Y - S - I - Aib - X₂ - E - K -
17 18 19 20 21 22 23 24 25 26 27 28 29 30 31
I - K - Q - Q - E - F - V - E - W - L - L - A - Q - K - P
32 33 34 35 36 37 38 39
S - S - G - A - P - P - P - S,

wherein X₁ is E, glutaric acid, adipic acid or succinic acid;

wherein X₂ is M, L or Nle;

or a functional variant thereof, wherein the amino acid sequence of said variant differs from SEQ ID NO:1 in that the amino acid sequence of the variant comprises 1, 2 or 3 individual amino acid substitutions,

wherein said peptide comprises a fatty acid molecule attached to the side chain amino group of the amino acid residue at position 18 of SEQ ID NO:1, or said functional variant thereof,

for use in a method of treating obesity, obesity-related disorders and/or dyslipidemia.

Also disclosed is a combination drug comprising i) a glucagon-like peptide-1 (GLP-1) receptor agonist, and ii) a glucose-dependent insulinotropic peptide receptor (GIPR) antagonist GIP peptide consisting of amino acid sequence SEQ ID NO:1:

(SEQ ID NO: 1)
3 - 4 - 5 - 6 7 8 9 10 11 12 13 14 15 16
X₁ - G - T - F - I - S - E - Y - S - I - Aib - X₂ - E - K -
17 18 19 20 21 22 23 24 25 26 27 28 29 30 31
I - K - Q - Q - E - F - V - E - W - L - L - A - Q - K - P
32 33 34 35 36 37 38 39
S - S - G - A - P - P - P - S,

wherein X₁ is E, glutaric acid, adipic acid or succinic acid;

wherein X₂ is M, L or Nle;

or a functional variant thereof, wherein the amino acid sequence of said variant differs from SEQ ID NO:1 in that the amino acid sequence of the variant comprises 1, 2 or 3 individual amino acid substitutions,

wherein said peptide comprises a fatty acid molecule attached to the side chain amino group of the amino acid residue at position 18 of SEQ ID NO:1, or said functional variant thereof,

for use in a method for inhibiting or reducing one or more of i) food intake, ii) body weight, iii) fasting blood levels of glucose, iv) fasting blood levels of insulin, v) insulin resistance, vi) fasting blood levels of triglycerides, vii) fasting blood levels of cholesterol and viii) fasting blood levels of low-density lipoprotein (LDL) cholesterol.

In one embodiment the GIPR antagonist GIP peptide and the GLP-1 receptor agonist of the present disclosure are contained together in the same composition or pharmaceutical formulation.

In one embodiment the GIPR antagonist GIP peptide and the GLP-1 receptor agonist of the present disclosure are each contained in separate compositions or pharmaceutical formulations.

In some embodiments, the GIPR antagonist GIP peptide and the GLP-1 receptor agonist are administered simultaneously.

In some embodiments, the GIPR antagonist GIP peptide and the GLP-1 receptor agonist are administered separately.

In some embodiments, the GIPR antagonist GIP peptide and the GLP-1 receptor agonist are administered sequentially.

### Monotherapy

Thus, one aspect of the present disclosure relates to a GIPR antagonist GIP peptide consisting of amino acid sequence SEQ ID NO:1:

(SEQ ID NO: 1)
3 - 4 - 5 - 6 7 8 9 10 11 12 13 14 15 16
X₁ - G - T - F - I - S - E - Y - S - I - Aib - X₂ - E - K -
17 18 19 20 21 22 23 24 25 26 27 28 29 30 31
I - K - Q - Q - E - F - V - E - W - L - L - A - Q - K - P
32 33 34 35 36 37 38 39
S - S - G - A - P - P - P - S,

wherein X₁ is E, glutaric acid, adipic acid or succinic acid;

wherein X₂ is M, L or Nle;

or a functional variant thereof, wherein the amino acid sequence of said variant differs from SEQ ID NO:1 in that the amino acid sequence of the variant comprises 1, 2 or 3 individual amino acid substitutions,

wherein said peptide comprises a fatty acid molecule attached to the side chain amino group of the amino acid residue at position 18 of SEQ ID NO:1, or said functional variant thereof,

for use in a method of inhibiting or reducing one or more of i) food intake, ii) body weight, iii) fasting blood levels of glucose, iv) fasting blood levels of insulin, v) insulin resistance, vi) fasting blood levels of triglycerides, vii) fasting blood levels of cholesterol and viii) fasting blood levels of low-density lipoprotein (LDL) cholesterol.

Another aspect of the present disclosure relates to a GIPR antagonist GIP peptide consisting of amino acid sequence SEQ ID NO:1:

(SEQ ID NO: 1)
3 - 4 - 5 - 6 7 8 9 10 11 12 13 14 15 16
X₁ - G - T - F - I - S - E - Y - S - I - Aib - X₂ - E - K -
17 18 19 20 21 22 23 24 25 26 27 28 29 30 31
I - K - Q - Q - E - F - V - E - W - L - L - A - Q - K - P
32 33 34 35 36 37 38 39
S - S - G - A - P - P - P - S,

wherein X₁ is E, glutaric acid, adipic acid or succinic acid;

wherein X₂ is M, L or Nle;

or a functional variant thereof, wherein the amino acid sequence of said variant differs from SEQ ID NO:1 in that the amino acid sequence of the variant comprises 1, 2 or 3 individual amino acid substitutions,

wherein said peptide comprises a fatty acid molecule attached to the side chain amino group of the amino acid residue at position 18 of SEQ ID NO:1, or said functional variant thereof,

for use in a method of treating obesity, obesity-related disorders and/or dyslipidemia.

In one embodiment, the peptide for use in a method of inhibiting or reducing any one of i) food intake, ii) body weight, iii) fasting blood levels of glucose, iv) fasting blood levels of insulin, v) insulin resistance, vi) fasting blood levels of triglycerides, vii) fasting blood levels of cholesterol and viii) fasting blood levels of low-density lipoprotein (LDL) cholesterol, and/or for use in a method of treating dyslipidemia, is a GIPR antagonist GIP peptide selected from the group consisting of:

i.
SEQ ID NO: 6
EGTFISEYSIAibLEKIKQQEFVEWLLAQKPSSGAP
PPS-C16-diacid/18K;
GIP(3-30)+Cex(31-39)
[D9E; A13Aib; M14L; D15E; H18K; D21E; N24E],
ii.
SEQ ID NO: 12
XGTFISEYSIAibNleEKIKQQEFVEWLLAQKPSSG
APPPS-2xAEEAc+yGlu-C18-diacid/18K;
GIP(3-30)+Cex(31-39)
[E3Glutaric acid(X); D9E; A13Aib;
M14Nle; D15E; H18K; D21E; N24E],
and
iii.
SEQ ID NO: 11
XGTFISEYSIAibLEKIKQQEFVEWLLAQKPSSGAPP
PS-C16-diacid/18K;
GIP(3-30)+Cex(31-39) [E3Glutaric acid(X);
D9E; A13Aib; M14L; D15E; H18K; D21E; N24E],

or a functional variant thereof comprising 1, 2 or 3 individual amino acid substitutions.

In one embodiment, the peptide for use in a method of inhibiting or reducing any one of i) food intake, ii) body weight, iii) fasting blood levels of glucose, iv) fasting blood levels of insulin, v) insulin resistance, vi) fasting blood levels of triglycerides, vii) fasting blood levels of cholesterol and viii) fasting blood levels of low-density lipoprotein (LDL) cholesterol, and/or for use in a method of treating dyslipidemia, is EGTFISEYSIAibLEKIKQQEFVEWLLAQKPSSGAPPPS-C16-diacid/18K; SEQ ID NO:6; GIP(3-30)+Cex(31-39) [D9E;A13Aib;M14L;D15E;H18K;D21E;N24E], or a functional variant thereof comprising 1, 2 or 3 individual amino acid substitutions.

In one embodiment, the peptide for use in a method of inhibiting or reducing any one of i) food intake, ii) body weight, iii) fasting blood levels of glucose, iv) fasting blood levels of insulin, v) insulin resistance, vi) fasting blood levels of triglycerides, vii) fasting blood levels of cholesterol and viii) fasting blood levels of low-density lipoprotein (LDL) cholesterol, and/or for use in a method of treating dyslipidemia, is XGTFISEYSIAibNIeEKIKQQEFVEWLLAQKPSSGAPPPS-2xAEEAc+yGlu-C18-diacid/18K; SEQ ID NO:12; GIP(3-30)+Cex(31-39) [E3Glutaric acid(X);D9E;A13Aib; M14Nle;D15E;H18K;D21E;N24E], or a functional variant thereof comprising 1, 2 or 3 individual amino acid substitutions.

In one embodiment, the peptide for use in a method of inhibiting or reducing any one of i) food intake, ii) body weight, iii) fasting blood levels of glucose, iv) fasting blood levels of insulin, v) insulin resistance, vi) fasting blood levels of triglycerides, vii) fasting blood levels of cholesterol and viii) fasting blood levels of low-density lipoprotein (LDL) cholesterol, and/or for use in a method of treating obesity, obesity-related disorders and/or dyslipidemia, is XGTFISEYSIAibLEKIKQQEFVEWLLAQKPSSGAPPPS-C16-diacid/18K; SEQ ID NO:11; GIP(3-30)+Cex(31-39) [E3Glutaric acid(X);D9E;A13Aib;M14L;D15E;H18K;D21E;N24E], or a functional variant thereof comprising 1, 2 or 3 individual amino acid substitutions.

### Patient Group

Overweight and obesity are defined as abnormal or excessive fat accumulation that presents a risk to health. A body mass index (BMI) over 25 is considered overweight, and over 30 is obese. Obesity is a complex disease involving an excessive amount of body fat. Obesity increases the risk of other diseases and health problems, such as heart disease, diabetes, high blood pressure and certain cancers.

An individual is usually considered to be obese if they have a body mass index (BMI) of 30 kg/m² or more, for example 35 kg/m² or more, such as 40 kg/m² or more. An individual is usually considered to be overweight if they have an BMI of 25 to <30.

In one embodiment of the present disclosure an individual is obese if they have a BMI of 25 kg/m² or more, such as 30 kg/m² or more, for example 35 kg/m² or more, such as 40 kg/m² or more.

In some embodiments of the present disclosure an individual is considered Class 1 obese if they have an BMI of 30 to <35. In some embodiments of the present disclosure an individual is considered Class 2 obese if they have an BMI of 35 to <40. In some embodiments of the present disclosure an individual is considered Class 3 obese if they have an BMI of >40. Class 3 obesity is sometimes categorized as “severe” obesity.

In one embodiment an individual is considered obese if they have a BMI of >25 and having one or more obesity-related disorders (co-morbidities).

In one embodiment an individual is considered to be obese if they have a waist circumference of ≥80 cm for women or of ≥94 cm for men.

In one embodiment the obesity-related disorder of the present disclosure is binge eating.

In one embodiment the obesity-related disorder of the present disclosure is selected from the group consisting of heart disease, stroke, high blood pressure, high blood cholesterol, high blood low-density lipoprotein (LDL) cholesterol, high blood triglycerides, dyslipidemia, gallbladder disease and gallstones, NAFLD (non-alcoholic fatty liver disease) and NASH (non-alcoholic steatohepatitis), osteoarthritis, gout, breathing problems such as sleep apnea and asthma, insulin resistance and diabetes mellitus.

In one embodiment the obesity-related disorder of the present disclosure is diabetes mellitus, Type II.

In one embodiment the obesity-related disorder of the present disclosure is dyslipidemia.

In one embodiment, the present disclosure provides a treatment for an obesity-related disorder in an individual who has normal body weight, in an individual who is overweight or in an individual who is obese. Individuals who are not obese or overweight may for other reasons be suffering from an obesity-related disorder such as those referred to herein including any one of heart disease, stroke, high blood pressure, high blood cholesterol, high blood low-density lipoprotein (LDL) cholesterol, high blood triglycerides, dyslipidemia, gallbladder disease and gallstones, NAFLD (non-alcoholic fatty liver disease) and NASH (non-alcoholic steatohepatitis), osteoarthritis, gout, breathing problems such as sleep apnea and asthma, insulin resistance and diabetes mellitus.

In one embodiment, the present disclosure provides a treatment, such as an optimized GIPR antagonist GIP peptide as defined herein alone or in combination with a GLP-1R agonist for use in treating one or more of heart disease, stroke, high blood pressure, high blood cholesterol, high blood low-density lipoprotein (LDL) cholesterol, high blood triglycerides, dyslipidemia, gallbladder disease and gallstones, NAFLD (non-alcoholic fatty liver disease) and NASH (non-alcoholic steatohepatitis), osteoarthritis, gout, breathing problems such as sleep apnea and asthma, insulin resistance and diabetes mellitus.

In one embodiment, the peptide(s) or composition for use according to the present disclosure is for treatment of an obesity-related disorder in an overweight individual.

In one embodiment, the peptide(s) or composition for use according to the present disclosure is for treatment of an obesity-related disorder in an obese individual.

In one embodiment, the peptide(s) or composition for use according to the present disclosure is for treatment of an obesity-related disorder in an individual with a BMI of 18 to 25.

In one embodiment, the peptide(s) or composition for use according to the present disclosure is for treatment of an obesity-related disorder in an individual with a BMI of >25.

In one embodiment, the peptide(s) or composition for use according to the present disclosure is for treatment of an obesity-related disorder in an individual with a BMI of 30 to <35.

In one embodiment, the peptide(s) or composition for use according to the present disclosure is for treatment of an obesity-related disorder in an individual with a BMI of 35 to <40.

In one embodiment, the peptide(s) or composition for use according to the present disclosure is for treatment of an obesity-related disorder in an individual with a BMI of >40.

In one embodiment, the peptide(s) or composition for use according to the present disclosure is for treatment of an obesity-related disorder in an individual with a waist circumference of ≥80 cm for women or of ≥94 cm for men.

### GIPR Antagonist GIP Peptides

The present disclosure in one embodiment concerns a GIPR antagonist GIP peptide consisting of amino acid sequence SEQ ID NO:1:

(SEQ ID NO: 1)
3 - 4 - 5 - 6 7 8 9 10 11 12 13 14 15 16
X₁ - G - T - F - I - S - E - Y - S - I - Aib - X₂ - E - K -
17 18 19 20 21 22 23 24 25 26 27 28 29 30 31
I - K - Q - Q - E - F - V - E - W - L - L - A - Q - K - P
32 33 34 35 36 37 38 39
S - S - G - A - P - P - P - S,

wherein X₁ is E, glutaric acid, adipic acid or succinic acid;

wherein X₂ is M, L or Nle;

or a functional variant thereof, wherein the amino acid sequence of said variant differs from SEQ ID NO:1 in that the amino acid sequence of the variant comprises 1, 2 or 3 individual amino acid substitutions,

wherein said peptide comprises a fatty acid molecule attached to the side chain amino group of the amino acid residue at position 18 of SEQ ID NO:1, or said functional variant thereof; in combinations and uses as disclosed herein.

The GIPR antagonist GIP peptides of the present disclosure have a sequence based on native human GIP3-30, and the numbering of the amino acid residues of the native hGIP has been kept for the GIPR antagonist GIP peptide. Thus, as provided herein the residue at position 3 is actually the first residue of the GIPR antagonist GIP peptides of the present disclosure (residue at position 1 in the official sequence listing). Similarly, the Lysine at position 18, which is used for attachment of a fatty acid, is actually the residue at position 16 in the official sequence listing.

The GIPR antagonist GIP peptide according to present disclosure are characterized by having higher solubility, higher physical stability and/or improved antagonistic properties compared to native hGIP, and as compared to hGIP3-30.

In some embodiments, the GIP peptide according to the present disclosure is such that X₁ of SEQ ID NO:1 is E.

In some embodiments, the GIP peptide according to the present disclosure is such that X₁ of SEQ ID NO:1 is glutaric acid.

In some embodiments, the GIP peptide according to the present disclosure, or the variant thereof, is such that X₂ of SEQ ID NO:1 is L.

In some embodiments, the GIP peptide according to the present disclosure, or the variant thereof, is such that X₂ of SEQ ID NO:1 is Ne.

In some embodiments, the GIP peptide according to the present disclosure, or the variant thereof, is such that X₁ of SEQ ID NO:1 is E and X₂ of SEQ ID NO:1 is L.

In some embodiments, the GIP peptide according to the present disclosure, or the variant thereof, is such that X₁ of SEQ ID NO:1 is glutaric acid and X₂ of SEQ ID NO:1 is L.

In some embodiments, the GIP peptide according to the present disclosure, or the variant thereof, is such that X₁ of SEQ ID NO:1 is glutaric acid and X₂ of SEQ ID NO:1 is Ne.

In some embodiments, the amino acid sequence of said functional variant of the GIP peptide according to the present disclosure differs from SEQ ID NO:1 in that the amino acid sequence of the variant comprises 3 individual amino acid substitutions.

In some embodiments, the amino acid sequence of said functional variant of the GIP peptide according to the present disclosure differs from SEQ ID NO:1 in that the amino acid sequence of the variant comprises 2 individual amino acid substitutions.

In some embodiments, the amino acid sequence of said functional variant of the GIP peptide according to the present disclosure differs from SEQ ID NO:1 in that the amino acid sequence of the variant comprises 1 amino acid substitution.

In some embodiments, the amino acid sequence of said functional variant of the GIP peptide according to the present disclosure differs from SEQ ID NO:1 in that the amino acid sequence of the variant comprises 1, 2 or 3 individual conservative amino acid substitutions.

In some embodiments, the amino acid sequence of said functional variant of the GIP peptide according to the present disclosure differs from SEQ ID NO:1 in that the amino acid sequence of the variant comprises 1, 2 or 3 individual amino acid substitutions at any one of positions 4 to 8, 10 to 13, 16, 17, 19, 20, 22, 23, 25 to 39 of SEQ ID NO:1.

In some embodiments, the GIPR antagonist GIP peptide according to the present disclosure is selected from the group consisting of:

SEQ ID NO: 1
EGTFISEYSAibANleEKIKQQDFVEWLLAQKPSSGAPPPS;
GIP(3-30)
[D9E; I12Aib; M14Nle; D15E; H18K; N24E],
SEQ ID NO: 2
EGTFISEYSIAibMEKIKQQDFVEWLLAQKPSSGAPPPS;
GIP(3-30)
[D9E; A13Aib; D15E; H18K; N24E],
SEQ ID NO: 3
EGTFISDYSIAibMDKIKQQDFVEWLLAQKPSSGAPPPS;
GIP(3-30)
[A13Aib; H18K; N24E],
SEQ ID NO: 4
EGTFISDYSIAibLDKIKQQDFVEWLLAQKPSSGAPPPS;
GIP(3-30)
[A13Aib; M14L; H18K; N24E],
SEQ ID NO: 5
EGTFISDYSIAibNleDKIKQQDFVEWLLAQKPSSGAPPPS;
GIP(3-30)
[A13Aib; M14Nle; H18K; N24E],
SEQ ID NO: 6
EGTFISEYSIAibLEKIKQQEFVEWLLAQKPSSGAPPPS;
GIP(3-30)
[D9E; A13Aib; M14L; D15E; H18K; D21E; N24E],
SEQ ID NO: 7
EGTFISEYSIAibNleEKIKQQEFVEWLLAQKPSSGAPPPS;
GIP(3-30)
[D9E; A13Aib; M14Nle; D15E; H18K; D21E; N24E],
SEQ ID NO: 8
EGTFISEYSIALEKIKQQEFVEWLLAQKPSSGAPPPS;
GIP(3-30)
[D9E; M14L; D15E; H18K; D21E; N24E],
SEQ ID NO: 9
EGTFISEYSIANleEKIKQQEFVEWLLAQKPSSGAPPPS;
GIP(3-30)
[D9E; M14Nle; D15E; H18K; D21E; N24E],
SEQ ID NO: 10
EGTFISEYSIAibLEKIKQQDFVEWLLAQKPSSGAPPPS;
GIP(3-30)
[D9E; A13Aib; M14L; D15E; H18K; N24E]
SEQ ID NO: 11
XGTFISEYSIAibLEKIKQQEFVEWLLAQKPSSGAPPPS;
GIP(3-30)
[E3Glutaric acid(X); D9E; A13Aib;
M14L; D15E; H18K; D21E; N24E],
SEQ ID NO: 12
XGTFISEYSIAibNleEKIKQQEFVEWLLAQKPSSGAPPPS;
GIP(3-30)
[E3Glutaric acid(X); D9E; A13Aib;
M14Nle; D15E; H18K; D21E; N24E],
SEQ ID NO: 13
XGTFISEYSIALEKIKQQEFVEWLLAQKPSSGAPPPS;
GIP(3-30)
[E3Glutaric acid(X); D9E;
M14L; D15E; H18K; D21E; N24E],
SEQ ID NO: 14
XGTFISEYSIAibMEKIKQQEFVEWLLAQKPSSGAPPPS;
GIP(3-30)
[E3Glutaric acid(X); D9E; A13Aib; D15E;
H18K; D21E; N24E],
SEQ ID NO: 15
EGTFISEYSIAibMEKIKQQEFVEWLLAQKPSSGAPPPS;
GIP(3-30)
[D9E; A13Aib; D15E; H18K; D21E; N24E],
SEQ ID NO: 16
EGTFISEYSIAMEKIKQQEFVEWLLAQKPSSGAPPPS;
GIP(3-30)
[D9E; D15E; H18K; D21E; N24E],
SEQ ID NO: 17
EGTFISEYSIAibLDKIKQQDFVEWLLAQKPSSGAPPPS;
GIP(3-30)
[D9E; A13Aib;
M14L; H18K; N24E]
SEQ ID NO: 18
XGTFISDYSIAibMDKIKQQDFVEWLLAQKPSSGAPPPS;
GIP(3-30)
[E3Glutaric acid(X); A13Aib; H18K; N24E],
SEQ ID NO: 2
EGTFISEYSIAibMEKIKQQDFVEWLLAQKPSSGAPPPS;
GIP(3-30)
[D9E,A13Aib; D15E; H18K; N24E],
SEQ ID NO: 20
XGTFISEYSIAibLEKIKQQEFVEWLLAQKPSSGAPPPS;
GIP(3-30)
[E3Succinic acid(X); D9E; A13Aib;
M14L; D15E; H18K; D21E; N24E],
and
SEQ ID NO: 21
XGTFISEYSIAibLEKIKQQEFVEWLLAQKPSSGAPPPS;
GIP(3-30)
[E3Adipic acid(X); D9E; A13Aib;
M14L; D15E; H18K; D21E; N24E],

or a functional variant thereof comprising 1, 2 or 3 individual amino acid substitutions.

In some embodiments, the GIPR antagonist GIP peptide according to the present disclosure is selected from the group consisting of:

SEQ ID NO: 6
EGTFISEYSIAibLEKIKQQEFVEWLLAQKPSSGAPPPS;
GIP(3-30)
[D9E; A13Aib; M14L; D15E; H18K; D21E; N24E],
SEQ ID NO: 11
XGTFISEYSIAibLEKIKQQEFVEWLLAQKPSSGAPPPS;
GIP(3-30)
[E3Glutaric acid(X); D9E; A13Aib;
M14L; D15E; H18K; D21E; N24E],
SEQ ID NO: 12
XGTFISEYSIAibNleEKIKQQEFVEWLLAQKPSSGAPPPS;
GIP(3-30)
[E3Glutaric acid(X); D9E; A13Aib; M14Nle; D15E;
H18K; D21E; N24E],

or a functional variant thereof comprising 1, 2 or 3 individual amino acid substitutions.

In some embodiments, the GIPR antagonist GIP peptide according to the present disclosure is C-terminally carboxylated (—COOH).

In some embodiments, the GIPR antagonist GIP peptide according to the present disclosure comprises a fatty acid, wherein the fatty acid molecule is a straight-chain fatty acid.

In some embodiments, the GIPR antagonist GIP peptide according to the present disclosure comprises a fatty acid, wherein the fatty acid molecule is a branched fatty acid.

In some embodiments, the GIPR antagonist GIP peptide according to the present disclosure comprises a fatty acid, wherein the fatty acid molecule is a diacyl fatty acid molecule.

In some embodiments, the GIPR antagonist GIP peptide according to the present disclosure comprises a fatty acid, wherein said fatty acid molecule comprises an acyl group of the formula CH₃(CH₂)ₙCO—, wherein n is in an integer from 4 to 24.

In some embodiments, the GIPR antagonist GIP peptide according to the present disclosure comprises a fatty acid, wherein said fatty acid molecule comprises one or more acyl groups selected from the group consisting of CH₃(CH₂)₆CO—, CH₃(CH₂)₈CO—, CH₃(CH₂)₁₀CO—, CH₃(CH₂)₁₂CO—, CH₃(CH₂)₁₄CO—, CH₃(CH₂)₁₆CO—, CH₃(CH₂)₁₈CO—, CH₃(CH₂)₂₀CO— and CH₃(CH₂)₂₂CO—.

In some embodiments, the GIPR antagonist GIP peptide according to the present disclosure comprises a fatty acid, wherein said fatty acid molecule comprises an acyl group selected from the group consisting of CH₃(CH₂)₁₀— (lauryl, C12), CH₃(CH₂)₁₂CO— (myristoyl, C14), CH₃(CH₂)₁₄CO— (palmitoyl, C16), CH₃(CH₂)₁₆CO— (stearyl, C18), CH₃(CH₂)₁₈CO— (arachidyl, C20) and CH₃(CH₂)₂₀CO— (behenyl, C22).

In some embodiments, the GIPR antagonist GIP peptide according to the present disclosure comprises a fatty acid, wherein said fatty acid molecule comprises two acyl groups individually selected from the group consisting of HOOC—CH₃(CH₂)₁₀CO— (dodecanoyl, C12), HOOC—CH₃(CH₂)₁₂CO— (1-tetradecanoyl, C14), HOOC—CH₃(CH₂)₁₄CO— (hexadecanoyl, C16), HOOC—CH₃(CH₂)₁₅CO— (15-carboxy-pentadecanoyl, C17), HOOC—CH₃(CH₂)₁₆CO— (octadecanoyl, C18), HOOC—CH₃(CH₂)₁₇CO— (17-carboxy-heptadecanoyl, C19), HOOC—CH₃(CH₂)₁₈CO— (eicosanoyl, C20), HOOC—CH₃(CH₂)₁₉CO— (19-carboxy-nonadecanoyl, C21) and HOOC—CH₃(CH₂)₂₀CO— (behenyl, C22).

In some embodiments, the GIPR antagonist GIP peptide according to the present disclosure comprises a fatty acid, wherein said fatty acid molecule comprises an acyl group of the formula COOH(CH₂)ₙCO— (dicarboxylic acid), wherein n is an integer from 4 to 24.

In some embodiments, the GIPR antagonist GIP peptide according to the present disclosure comprises a fatty acid, wherein said fatty acid molecule comprises an acyl group selected from the group consisting of COOH(CH₂)₁₄CO—, COOH(CH₂)₁₆CO—, COOH(CH₂)₁₈CO— and COOH(CH₂)₂₀CO—.

In some embodiments, the GIPR antagonist GIP peptide according to the present disclosure comprises a fatty acid, wherein said fatty acid molecule comprises or consists of COOH(CH₂)₁₄CO—.

In some embodiments, the GIPR antagonist GIP peptide according to the present disclosure comprises a fatty acid, wherein said fatty acid molecule comprises or consists of COOH(CH₂)₁₆CO—.

In some embodiments, the GIPR antagonist GIP peptide according to the present disclosure comprises a fatty acid, wherein said fatty acid molecule comprises or consists of COOH(CH₂)₁₈CO—.

In some embodiments, the GIPR antagonist GIP peptide according to the present disclosure comprises a fatty acid, wherein said fatty acid molecule is attached to the side chain amino group of a lysine residue at position 18 of SEQ ID NO:1, or a variant of SEQ ID NO:1.

In some embodiments, the GIPR antagonist GIP peptide according to the present disclosure comprises a fatty acid, wherein said fatty acid molecule is attached to the side chain amino group of an ornithine residue at position 18 of SEQ ID NO:1, or a variant of SEQ ID NO:1.

In some embodiments, the GIPR antagonist GIP peptide according to the present disclosure comprises a fatty acid, wherein said fatty acid molecule is attached to the side chain amino group of the amino acid residue at position 18 of SEQ ID NO:1, or a variant of SEQ ID NO:1, via a linker.

In some embodiments, the GIPR antagonist GIP peptide according to the present disclosure comprises a fatty acid, wherein the fatty acid molecule is attached to an amino acid residue of SEQ ID NO:1, or a variant thereof, via a linker in such a way that a carboxyl group of the fatty acid molecule forms an amide bond with an amino group of the linker.

In some embodiments, the GIPR antagonist GIP peptide according to the present disclosure comprises a fatty acid molecule attached to an amino acid residue of SEQ ID NO:1, or a variant thereof, via a linker, wherein said linker comprises one or more moieties individually selected from the group consisting of:
a. α-amino acid, γ-amino acid or w-amino acid,
b. one or more amino acids selected from the group consisting of succinic acid, Lys, Glu, Asp,
c. one or more amino acids selected from the group consisting of Gly and Ser,
d. one or more amino acids selected from the group consisting of Ala, Glu, Lys and Leu,
e. one or more of γ-aminobutanoyl (γ-aminobutyric acid), γ-Glu (γ-glutamic acid), β-Asp (β-asparagyl), β-Ala (β-alanyl), 2-aminoisobutyric acid (Aib) and Gly, and
f. [8-amino-3,6-dioxaoctanoic acid]n (AEEAcₙ), wherein n is an integer between 1 and 50, such as an integer between 1-4, 1-3 or 1-2.

In some embodiments, the GIPR antagonist GIP peptide according to the present disclosure comprises a fatty acid molecule attached to an amino acid residue of SEQ ID NO:1, or a variant thereof, via a linker, wherein said linker comprises a γ-Glu, one or more 8-amino-3,6-dioxaoctanoic acid (AEEAc), or combinations thereof, for example wherein said linker comprises or consists of a [8-amino-3,6-dioxaoctanoic acid]ₙ (AEEAc)ₙ, wherein n is an integer between 1 and 50, such as an integer between 1-2, 2-3, 3-4, 4-5, 5-6, 6-7, 7-8, 8-9, 9-10, 10-11, 11-12, 12-13, 13-14, 14-15, 15-20, 20-25, 25-30, 30-35, 35-40, 40-45, 45-50, preferably wherein n is 1, 2 or 3.

In some embodiments, the GIPR antagonist GIP peptide according to the present disclosure comprises a fatty acid molecule attached to an amino acid residue of SEQ ID NO:1, or a variant thereof, via a linker, wherein said linker comprises or consists of a γ-Glu and two AEEAc.

In some embodiments, the GIPR antagonist GIP peptide according to the present disclosure comprises a fatty acid molecule attached to an amino acid residue of SEQ ID NO:1, or a variant thereof, via a linker, wherein said linker comprises or consists of γ-Glu-AEEAc-AEEAc.

In some embodiments, the GIPR antagonist GIP peptide according to the present disclosure comprises a fatty acid, wherein the fatty acid molecule is attached to the side chain amino group of the amino acid residue at position 18 of SEQ ID NO:1, or said functional variant thereof, via a linker, and wherein the combination of linker and fatty acid molecule is selected from the group consisting of:
i. Hexadecanoyl-γ-Glu-
ii. Hexadecanoyl-γ-Glu-γ-Glu-
iii. Hexadecanoyl-γ-Glu-AEEAc-
iv. Hexadecanoyl-γ-Glu-AEEAc-AEEAc-
v. Hexadecanoyl-γ-Glu-AEEAc-AEEAc-AEEAc-
vi. [15-carboxy-pentadecanoyl]-γ-Glu-
vii. [15-carboxy-pentadecanoyl]-γ-Glu-γ-Glu-
viii. [15-carboxy-pentadecanoyl]-γ-Glu-AEEAc-
ix. [15-carboxy-pentadecanoyl]-γ-Glu-AEEAc-AEEAc-
x. [15-carboxy-pentadecanoyl]-γ-Glu-AEEAc-AEEAc-AEEAc-
xi. Octadecanoyl-γ-Glu-
xii. Octadecanoyl-γ-Glu-γ-Glu-
xiii. Octadecanoyl-γ-Glu-AEEAc-
xiv. Octadecanoyl-γ-Glu-AEEAc-AEEAc-
xv. Octadecanoyl-γ-Glu-AEEAc-AEEAc-AEEAc-
xvi. [17-carboxy-heptadecanoyl]-γ-Glu-
xvii. [17-carboxy-heptadecanoyl]-γ-Glu-γ-Glu-
xviii. [17-carboxy-heptadecanoyl]-γ-Glu-AEEAc-
xix. [17-carboxy-heptadecanoyl]-γ-Glu-AEEAc-AEEAc-
xx. [17-carboxy-heptadecanoyl]-γ-Glu-AEEAc-AEEAc-AEEAc-
xxi. Eicosanoyl-γ-Glu-
xxii. Eicosanoyl-γ-Glu-γ-Glu-
xxiii. Eicosanoyl-γ-Glu-AEEAc-
xxiv. Eicosanoyl-γ-Glu-AEEAc-AEEAc-
xxv. Eicosanoyl-γ-Glu-AEEAc-AEEAc-AEEAc-
xxvi. [19-carboxy-nonadecanoyl]-γ-Glu-
xxvii. [19-carboxy-nonadecanoyl]-γ-Glu-γ-Glu-
xxviii. [19-carboxy-nonadecanoyl]-γ-Glu-AEEAc-
xxix. [19-carboxy-nonadecanoyl]-γ-Glu-AEEAc-AEEAc-, and
xxx. [19-carboxy-nonadecanoyl]-γ-Glu-AEEAc-AEEAc-AEEAc-.

In some embodiments, the GIPR antagonist GIP peptide according to the present disclosure comprises a fatty acid, wherein the fatty acid molecule is attached to the side chain amino group of the amino acid residue at position 18 of SEQ ID NO:1, or said functional variant thereof, via a linker, and wherein the combination of linker and fatty acid molecule is [17-carboxy-heptadecanoyl]-γ-Glu-AEEAc-AEEAc-.

In some embodiments, the GIPR antagonist GIP peptide according to the present disclosure comprises a fatty acid, wherein the fatty acid molecule is attached to the side chain amino group of the amino acid residue at position 18 of SEQ ID NO:1, or said functional variant thereof, directly, that is without a linker or spacer, and wherein the fatty acid is selected from the group consisting of:
i. [15-Carboxy pentadecanoyl], and
ii. [17-carboxy-heptadecanoyl].

In some embodiments, the GIPR antagonist GIP peptide according to the present disclosure is selected from the group consisting of:

SEQ ID NO: 38
EGTFISEYSIAMEKIKQQDFVEWLLAQKPSSGAPPPS
-C16-diacid/18K;
GIP(3-30)+Cex(31-39)
[D9E; D15E; H18K; N24E],
SEQ ID NO: 1
EGTFISEYSAibANleEKIKQQDFVEWLLAQKPSSGA
PPPS-C16-diacid/18K;
GIP(3-30)+Cex(31-39)
[D9E; I12Aib; M14Nle; D15E; H18K; N24E],
SEQ ID NO: 2
EGTFISEYSIAibMEKIKQQDFVEWLLAQKPSSGAPPPS
-C16-diacid/18K;
GIP(3-30)+Cex(31-39)
[D9E; A13Aib; D15E; H18K; N24E],
SEQ ID NO: 3
EGTFISDYSIAibMDKIKQQDFVEWLLAQKPSSGAPPPS
-C16-diacid/18K;
GIP(3-30)+Cex(31-39)
[A13Aib; H18K; N24E],
SEQ ID NO: 4
EGTFISDYSIAibLDKIKQQDFVEWLLAQKPSSGAPPPS
-C16-diacid/18K;
GIP(3-30)+Cex(31-39)
[A13Aib; M14L; H18K; N24E],
SEQ ID NO: 4
EGTFISDYSIAibLDKIKQQDFVEWLLAQKPSSGAPPPS-
2xAEEAc+yGlu-C18-diacid/18K;
GIP(3-30)+Cex(31-39)
[A13Aib; M14L; H18K; N24E],
SEQ ID NO: 5
EGTFISDYSIAibNleDKIKQQDFVEWLLAQKPSSGAPPPS
-C16-diacid/18K;
GIP(3-30)+Cex(31-39)
[A13Aib; M14Nle; H18K; N24E],
SEQ ID NO: 5
EGTFISDYSIAibNleDKIKQQDFVEWLLAQKPSSGAPPPS-
2xAEEAc+yGlu-C18-diacid/18K;
GIP(3-30)+Cex(31-39)
[A13Aib; M14Nle; H18K; N24E],
SEQ ID NO: 6
EGTFISEYSIAibLEKIKQQEFVEWLLAQKPSSGAPPPS
-C16-diacid/18K;
GIP(3-30)+Cex(31-39)
[D9E; A13Aib; M14L; D15E; H18K; D21E; N24E],
SEQ ID NO: 6
EGTFISEYSIAibLEKIKQQEFVEWLLAQKPSSGAPPPS-
2xAEEAc+yGlu-C18-diacid/18K;
GIP(3-30)+Cex(31-39)
[D9E; A13Aib; M14L; D15E; H18K; D21E; N24E],
SEQ ID NO: 7
EGTFISEYSIAibNleEKIKQQEFVEWLLAQKPSSGAPPPS
-C16-diacid/18K;
GIP(3-30)+Cex(31-39)
[D9E; A13Aib; M14Nle; D15E; H18K; D21E; N24E],
SEQ ID NO: 7
EGTFISEYSIAibNleEKIKQQEFVEWLLAQKPSSGAPPPS-
2xAEEAc+yGlu-C18-diacid/18K;
GIP(3-30)+Cex(31-39)
[D9E; A13Aib; M14Nle; D15E; H18K; D21E; N24E],
SEQ ID NO: 8
EGTFISEYSIALEKIKQQEFVEWLLAQKPSSGAPPPS-
2xAEEAc+yGlu-C18-diacid/18K;
GIP(3-30)+Cex(31-39)
[D9E; M14L; D15E; H18K; D21E; N24E],
SEQ ID NO: 9
EGTFISEYSIANleEKIKQQEFVEWLLAQKPSSGAPPPS-
2xAEEAc+yGlu-C18-diacid/18K;
GIP(3-30)+Cex(31-39)
[D9E; M14Nle; D15E; H18K; D21EN24E],
SEQ ID NO: 11
XGTFISEYSIAibLEKIKQQEFVEWLLAQKPSSGAPPPS-
2xAEEAc+yGlu-C18-diacid/18K;
GIP(3-30)+Cex(31-39)
[E3Glutaric acid(X); D9E; A13Aib;
M14L; D15E; H18K; D21E; N24E],
SEQ ID NO: 12
XGTFISEYSIAibNleEKIKQQEFVEWLLAQKPSSGAPPPS-
2xAEEAc+yGlu-C18-diacid/18K;
GIP(3-30)+Cex(31-39)
[E3Glutaric acid(X); D9E; A13Aib;
M14Nle; D15E; H18K; D21E; N24E],
SEQ ID NO: 13
XGTFISEYSIALEKIKQQEFVEWLLAQKPSSGAPPPS-OH-
2xAEEAc+yGlu-C18-diacid/18K;
GIP(3-30)+Cex(31-39)
[E3Glutaric acid(X); D9E;
M14L; D15E; H18K; D21E; N24E],
SEQ ID NO: 14
XGTFISEYSIAibMEKIKQQEFVEWLLAQKPSSGAPPPS-
2xAEEAc+yGlu-C18-diacid/18K;
GIP(3-30)+Cex(31-39)
[E3Glutaric acid(X);
D9E; A13Aib; D15E; H18K; D21E; N24E],
SEQ ID NO: 15
EGTFISEYSIAibMEKIKQQEFVEWLLAQKPSSGAPPPS-
2xAEEAc+yGlu-C18-diacid/18K;
GIP(3-30)+Cex(31-39)
[D9E; A13Aib; D15E; H18K; D21E; N24E],
SEQ ID NO: 16
EGTFISEYSIAMEKIKQQEFVEWLLAQKPSSGAPPPS-
2xAEEAc+yGlu-C18-diacid/18K;
GIP(3-30)+Cex(31-39)
[D9E; D15E; H18K; D21E; N24E],
SEQ ID NO: 17
EGTFISEYSIAibLDKIKQQDFVEWLLAQKPSSGAPPPS-
2xAEEAc+yGlu-C18-diacid/18K;
GIP(3-30)+Cex(31-39)
[D9E; A13Aib;
M14L; H18K; N24E],
SEQ ID NO: 11
XGTFISEYSIAibLEKIKQQEFVEWLLAQKPSSGAPPPS-
2xAEEAc+yGlu-C18-diacid/18K;
GIP(3-30)+Cex(31-39)
[E3Glutaric acid(X);
D9E; A13Aib; M14L; D15E; H18K; D21E; N24E],
SEQ ID NO: 6
EGTFISEYSIAibLEKIKQQEFVEWLLAQKPSSGAPPPS-
2xAEEAc+yGlu-C18-diacid/18K;
GIP(3-30)+Cex(31-39)
[D9E; A13Aib; M14L; D15E; H18K; D21E; N24E],
SEQ ID NO: 18
XGTFISDYSIAibMDKIKQQDFVEWLLAQKPSSGAPPPS
-C16-diacid/18K;
GIP(3-30)+Cex(31-39)
[E3Glutaric acid(X); A13Aib; H18K; N24E],
SEQ ID NO: 2
EGTFISEYSIAibMEKIKQQDFVEWLLAQKPSSGAPPPS
-C16-diacid/18K;
GIP(3-30)+Cex(31-39)
[D9E; A13Aib; D15E; H18K; N24E],
SEQ ID NO: 11
XGTFISEYSIAibLEKIKQQEFVEWLLAQKPSSGAPPPS
-C16-diacid/18K;
GIP(3-30)+Cex(31-39)
[E3Glutaric acid(X);
D9E; A13Aib; M14L; D15E; H18K; D21E; N24E],
SEQ ID NO: 8
EGTFISEYSIALEKIKQQEFVEWLLAQKPSSGAPPPS
-C16-diacid/18K;
GIP(3-30)+Cex(31-39)
[D9E; M14L; D15E; H18K; N24E],
SEQ ID NO: 6
EGTFISEYSIAibLEKIKQQEFVEWLLAQKPSSGAPPPS-
(GGGS-C16-diacid/18K);
GIP(3-30)+Cex(31-39)
[D9E; A13Aib; M14L; D15E; H18K; D21E; N24E],
SEQ ID NO: 6
EGTFISEYSIAibLEKIKQQEFVEWLLAQKPSSGAPPPS-
(ALEA-C16-diacid/18K);
GIP(3-30)+Cex(31-39)
[D9E; A13Aib; M14L; D15E; H18K; D21E; N24E],
SEQ ID NO: 10
EGTFISEYSIAibLEKIKQQDFVEWLLAQKPSSGAPPPS-
2xAEEAc+yGlu-C18-diacid/18K;
GIP(3-30)+Cex(31-39)
[D9E; A13Aib;
M14L; D15E; H18K; N24E],
SEQ ID NO: 12
XGTFISEYSIAibNleEKIKQQEFVEWLLAQKPSSGAPPPS
-C16-diacid/18K);
GIP(3-30)+Cex(31-39)
[E3Glutaric acid(X);
D9E; A13Aib; M14Nle; D15E; H18K; D21E; N24E],
SEQ ID NO: 6
EGTFISEYSIAibLEKIKQQEFVEWLLAQKPSSGAPPPS-
(Aib-C16-diacid/18K);
GIP(3-30)+Cex(31-39)
[D9E; A13Aib; M14L; D15E; H18K; D21E; N24E],
SEQ ID NO: 6
EGTFISEYSIAibLEKIKQQEFVEWLLAQKPSSGAPPPS-
(KAAAEKAAAEKAAAE
-C16-diacid/18K);
GIP(3-30)+Cex(31-39)
[D9E; A13Aib; M14L; D15E; H18K; D21E; N24E],
SEQ ID NO: 20
XGTFISEYSIAibLEKIKQQEFVEWLLAQKPSSGAPPPS-
2xAEEAc+yGlu-C18-diacid/18K;
GIP(3-30)+Cex(31-39)
[E3Succinic acid(X);
D9E; A13Aib; M14L; D15E; H18K; D21E; N24E],
and
SEQ ID NO: 21
XGTFISEYSIAibLEKIKQQEFVEWLLAQKPSSGAPPPS-
2xAEEAc+yGlu-C18-diacid/18K;
GIP(3-30)+Cex(31-39)
[E3Adipic acid(X); D9E; A13Aib;
M14L; D15E; H18K; D21E; N24E],

or

a functional variant thereof comprising 1, 2 or 3 individual amino acid substitutions.

In particular embodiments, the GIPR antagonist GIP peptide according to the present disclosure is selected from the group consisting of:

SEQ ID NO: 6

| | |
|---|---|
| i. | EGTFISEYSIAibLEKIKQQEFVEWLLAQKPSSGAPPPS-C16- |
| | diacid/18K; GIP(3-30) + Cex(31-39) |
| | [D9E; A13Aib; M14L; D15E; H18K; D21E; N24E], |

SEQ ID NO: 12

| | |
|---|---|
| ii. | XGTFISEYSIAibNleEKIKQQEFVEWLLAQKPSSGAPPPS- |
| | 2xAEEAc + yGlu-C18-diacid/18K; GIP(3-30) + |
| | Cex(31-39)[E3Glutaric acid(X); D9E; A13Aib; |
| | M14Nle; D15E; H18K; D21E; N24E], |

and
SEQ ID NO: 11

| | |
|---|---|
| iii. | XGTFISEYSIAibLEKIKQQEFVEWLLAQKPSSGAPPPS- |
| | C16-diacid/18K; GIP(3-30) + Cex(31-39) |
| | [E3Glutaric acid(X); D9E; A13Aib; M14L; |
| | D15E; H18K; D21E; N24E], |

or a functional variant thereof comprising 1, 2 or 3 individual amino acid substitutions.

In a particular embodiment, the GIPR antagonist GIP peptide according to the present disclosure is: EGTFISEYSIAibLEKIKQQEFVEWLLAQKPSSGAPPPS-C16-diacid/18K; SEQ ID NO:6; GIP(3-30)+Cex(31-39) [D9E;A13Aib;M14L;D15E;H18K;D21E;N24E], or a functional variant thereof comprising 1, 2 or 3 individual amino acid substitutions.

In a particular embodiment, the GIPR antagonist GIP peptide according to the present disclosure is: XGTFISEYSIAibNIeEKIKQQEFVEWLLAQKPSSGAPPPS-2xAEEAc+yGlu-C18-diacid/18K; SEQ ID NO:12; GIP(3-30)+Cex(31-39) [E3Glutaric acid(X);D9E;A13Aib; M14Nle;D15E;H18K;D21E;N24E], or a functional variant thereof comprising 1, 2 or 3 individual amino acid substitutions.

In a particular embodiment, the GIPR antagonist GIP peptide according to the present disclosure is: XGTFISEYSIAibLEKIKQQEFVEWLLAQKPSSGAPPPS-C16-diacid/18K; SEQ ID NO: 11; GIP(3-30)+Cex(31-39) [E3Glutaric acid(X);D9E;A13Aib;M14L;D15E; H18K;D21E;N24E], or a functional variant thereof comprising 1, 2 or 3 individual amino acid substitutions.

### GLP-1Receptor Agonists

In some embodiments the GIPR antagonist GIP peptide according to the present disclosure is co-administered with a GLP-1 receptor (GLP-1R) agonist.

In some embodiments of the present disclosure, said GLP-1 receptor agonist is a non-peptide GLP-1 receptor agonist.

In some embodiments of the present disclosure, said GLP-1 receptor agonist is a small molecule GLP-1R agonist.

These include cyclobutane-derivative-based non-peptidic agonists for GLP-1R, including Boc5 and its newly discovered analogue WB4-24.

In some embodiments of the present disclosure, said small molecule GLP-1 receptor agonist is selected from the group consisting of Boc5, WB4-24, OWL833, TT-OAD2, LY3502970 and PF 06882961.

In some embodiments of the present disclosure, said GLP-1 receptor agonist is a GLP-1 receptor activating antibody.

In some embodiments said GLP-1R agonist is an agonist of the GLP-1 receptor. In some embodiments said GLP-1R agonist is an agonist of only the GLP-1 receptor. some embodiments said GLP-1R agonist is an agonist of mainly the GLP-1 receptor. some embodiments said GLP-1R agonist is an agonist of the GLP-1 receptor, and one or more additional receptors.

In some embodiments of the present disclosure, said GLP-1 receptor agonist is a dual acting GLP-1 receptor agonist. In some embodiments of the present disclosure, said GLP-1 receptor agonist is a dual agonist of the GLP-1 receptor and an additional receptor. These may be peptide-based or non-peptide based.

In some embodiments of the present disclosure, said GLP-1 receptor agonist is a triple acting GLP-1 receptor agonist. In some embodiments of the present disclosure, said GLP-1 receptor agonist is a triple agonist of the GLP-1 receptor and two additional receptors.

In some embodiments of the present disclosure, said GLP-1 receptor agonist is a GLP-1R/glucagon dual agonist.

In some embodiments of the present disclosure, said GLP-1 receptor agonist is a peptide GLP-1 receptor agonist.

In some embodiments of the present disclosure, said GLP-1 receptor agonist is a GLP-1 peptide.

In some embodiments of the present disclosure, said GLP-1 peptide is a protease-resistant analogue of hGLP-1 (human GLP-1; SEQ ID NO:32).

In some embodiments of the present disclosure, said GLP-1 peptide is selected from the group consisting of exenatide (SEQ ID NO:22), Lixisenatide (SEQ ID NO:23), albiglutide (SEQ ID NO:24), liraglutide (SEQ ID NO:25), taspoglutide (SEQ ID NO:26), dulaglutide (SEQ ID NO:27), semaglutide (SEQ ID NO:19), efpeglenatide, exendin-4 (Ex4; SEQ ID NO:22), Ex4(1-30) (SEQ ID NO:29), Ex4(9-39) (SEQ ID NO:30), Ex(9-30) (SEQ ID NO:28), hGLP-1(1-37) (SEQ ID NO:32), hGLP-1(7-36) (SEQ ID NO:33), hGLP-1(7-37) (SEQ ID NO:34), hGLP-1(1-36) (SEQ ID NO:35), hGLP-1(9-36) (SEQ ID NO:36), A7-hGLP-1(7-36) (SEQ ID NO:37) and A10-hGLP-1(7-36) (SEQ ID NO:28), or a functional variant thereof.

In some embodiments of the present disclosure, said GLP-1 peptide is hGLP-1(7-37), or a variant thereof, such as a variant having one or two individual amino acid substitutions, such as a variant having two individual amino acid substitutions, and optionally comprising a fatty acid molecule. In one embodiment said variant has one or two individual amino acid substitutions at positions 8 and 34.

In some embodiments of the present disclosure, said GLP-1 peptide is (Arg34)hGLP-1(7-37):

7 8 9 10 11 12 13 14 15 16 17 18 19 20 21 22 H-A-E-G-T-F-T-S-D-V-S-S-Y-L-E-G-23 24 25 26 27 28 29 30 31 32 33 34 35 36 37 Q-A-A-K-E-F-I-A-W-L-V-R-G-R-G (SEQ ID NO: 25), or a variant thereof, such as variant having one individual amino acid substitution, and optionally comprising a fatty acid molecule; or such as a variant comprising a fatty acid molecule.

In some embodiments of the present disclosure, said GLP-1 peptide is (Arg34)hGLP-1(7-37) comprising a fatty acid molecule attached to the lysine at position 26, or a variant thereof, such as a variant having one amino acid substitution. In one embodiment said variant has an amino acid substitution at position 8.

In one embodiment of the present disclosure, said GLP-1 peptide is (Arg34)hGLP-1(7-37) comprising a fatty acid molecule attached to the lysine at position 26.

In some embodiments of the present disclosure, said GLP-1 peptide is liraglutide.

Liraglutide is a GLP-1 receptor agonist and its chemical structure is N26-(Hexadecanoyl-gamma-glutamyle)-(Arg34)GLP-1-(7-37)-peptide.

In one embodiment of the present disclosure, said GLP-1 peptide is (Aib8)(Arg34)hGLP-1(7-37) comprising a fatty acid molecule attached to the lysine at position 26.

In a particular embodiment of the present disclosure, said GLP-1 peptide is semaglutide.

In some embodiments of the present disclosure, said GLP-1 peptide is a liquid formulation of semaglutide.

In some embodiments of the present disclosure, said GLP-1 peptide is a solid oral dosage form composition of semaglutide.

Semaglutide is a GLP-1 receptor agonist and its chemical structure is N-epsilon26-[2-(2-{2-[2-(2-{2-[(S)-4-carboxy-4-(17-carboxyheptadecanoylamino)butyrylamino]ethoxy}ethoxy)acetylamino]ethoxy}ethoxy)a cetyl][Aib8,Arg34]GLP-1(7-37) as disclosed in e.g. U.S. Pat. No. 8,129,343.

The amino acid sequence of semaglutide differs from that of human GLP-1 because it is a truncated analogue, and has two amino acid substitutions at positions 8 and 34, and the lysine at position 26 is acylated with stearic diacid (C-18 fatty diacid chain) via a spacer, corresponding to modified residue: N-epsilon26-[2-(2-{2-[2-(2-{2-[(S)-4-carboxy-4-(17-carboxyheptadecanoylamino)butyrylamino]ethoxy}ethoxy)acetylamino]ethoxy}ethoxy)a cetyl].

### Combinations

In one embodiment, the GIPR antagonist GIP peptide of the present disclosure is selected from the group consisting of: EGTFISEYSIAibLEKIKQQEFVEWLLAQKPSSGAPPPS-C16-diacid/18K; SEQ ID NO:6; GIP(3-30)+Cex(31-39) [D9E;A13Aib;M14L;D15E;H18K;D21E;N24E], XGTFISEYSIAibNIeEKIKQQEFVEWLLAQKPSSGAPPPS-2xAEEAc+yGlu-C18-diacid/18K; SEQ ID NO:12; GIP(3-30)+Cex(31-39) [E3Glutaric acid(X);D9E;A13Aib; M14Nle;D15E;H18K;D21E;N24E], and XGTFISEYSIAibLEKIKQQEFVEWLLAQKPSSGAPPPS-C16-diacid/18K; SEQ ID NO:11; GIP(3-30)+Cex(31-39) [E3Glutaric acid(X);D9E;A13Aib;M14L;D15E;H18K;D21E;N24E], or a functional variant thereof comprising 1, 2 or 3 individual amino acid substitutions;

and the GLP-1 receptor agonist is (Arg34)hGLP-1(7-37) (SEQ ID NO:25),

or a variant thereof, such as variant having one individual amino acid substitution, and optionally comprising a fatty acid molecule; or such as a variant comprising a fatty acid molecule.

In a particular embodiment, the GIPR antagonist GIP peptide is selected from the group consisting of: EGTFISEYSIAibLEKIKQQEFVEWLLAQKPSSGAPPPS-C16-diacid/18K; SEQ ID NO:6; GIP(3-30)+Cex(31-39) [D9E;A13Aib;M14L;D15E;H18K;D21E;N24E], XGTFISEYSIAibNIeEKIKQQEFVEWLLAQKPSSGAPPPS-2xAEEAc+yGlu-C18-diacid/18K; SEQ ID NO:12; GIP(3-30)+Cex(31-39) [E3Glutaric acid(X);D9E;A13Aib; M14Nle;D15E;H18K;D21E;N24E], and XGTFISEYSIAibLEKIKQQEFVEWLLAQKPSSGAPPPS-C16-diacid/18K; SEQ ID NO:11; GIP(3-30)+Cex(31-39) [E3Glutaric acid(X);D9E;A13Aib;M14L;D15E;H18K;D21E;N24E], or a functional variant thereof comprising 1, 2 or 3 individual amino acid substitutions;

and the GLP-1 receptor agonist is (Arg34)hGLP-1(7-37) (SEQ ID NO:25) comprising a fatty acid molecule attached to the lysine at position 26 of said (SEQ ID NO:25);

or a variant thereof, such as a variant having one individual amino acid substitution. In one embodiment said variant has an amino acid substitution at position 8.

In a particular embodiment, the GIPR antagonist GIP peptide is selected from the group consisting of: EGTFISEYSIAibLEKIKQQEFVEWLLAQKPSSGAPPPS-C16-diacid/18K; SEQ ID NO:6; GIP(3-30)+Cex(31-39) [D9E;A13Aib;M14L;D15E;H18K;D21E;N24E], XGTFISEYSIAibNIeEKIKQQEFVEWLLAQKPSSGAPPPS-2xAEEAc+yGlu-C18-diacid/18K; SEQ ID NO:12; GIP(3-30)+Cex(31-39) [E3Glutaric acid(X);D9E;A13Aib; M14Nle;D15E;H18K;D21E;N24E], and XGTFISEYSIAibLEKIKQQEFVEWLLAQKPSSGAPPPS-C16-diacid/18K; SEQ ID NO:11; GIP(3-30)+Cex(31-39) [E3Glutaric acid(X);D9E;A13Aib;M14L;D15E;H18K;D21E;N24E], or a functional variant thereof comprising 1, 2 or 3 individual amino acid substitutions;

and the GLP-1 receptor agonist is liraglutide or semaglutide.

In a particular embodiment, the GIPR antagonist GIP peptide is selected from the group consisting of: EGTFISEYSIAibLEKIKQQEFVEWLLAQKPSSGAPPPS-C16-diacid/18K; SEQ ID NO:6; GIP(3-30)+Cex(31-39) [D9E;A13Aib;M14L;D15E;H18K;D21E;N24E], XGTFISEYSIAibNIeEKIKQQEFVEWLLAQKPSSGAPPPS-2xAEEAc+yGlu-C18-diacid/18K; SEQ ID NO:12; GIP(3-30)+Cex(31-39) [E3Glutaric acid(X);D9E;A13Aib; M14Nle;D15E;H18K;D21E;N24E], and XGTFISEYSIAibLEKIKQQEFVEWLLAQKPSSGAPPPS-C16-diacid/18K; SEQ ID NO:11; GIP(3-30)+Cex(31-39) [E3Glutaric acid(X);D9E;A13Aib;M14L;D15E;H18K;D21E;N24E], or a functional variant thereof comprising 1, 2 or 3 individual amino acid substitutions;

and the GLP-1 receptor agonist is semaglutide.

In a particular embodiment, the GIPR antagonist GIP peptide is selected from the group consisting of: EGTFISEYSIAibLEKIKQQEFVEWLLAQKPSSGAPPPS-C16-diacid/18K; SEQ ID NO:6; GIP(3-30)+Cex(31-39) [D9E;A13Aib;M14L;D15E;H18K;D21E;N24E], XGTFISEYSIAibNIeEKIKQQEFVEWLLAQKPSSGAPPPS-2xAEEAc+yGlu-C18-diacid/18K; SEQ ID NO:12; GIP(3-30)+Cex(31-39) [E3Glutaric acid(X);D9E;A13Aib; M14Nle;D15E;H18K;D21E;N24E], and XGTFISEYSIAibLEKIKQQEFVEWLLAQKPSSGAPPPS-C16-diacid/18K; SEQ ID NO:11; GIP(3-30)+Cex(31-39) [E3Glutaric acid(X);D9E;A13Aib;M14L;D15E;H18K;D21E;N24E], or a functional variant thereof comprising 1, 2 or 3 individual amino acid substitutions;

and wherein the GLP-1 receptor agonist is (Arg34)hGLP-1(7-37) (SEQ ID NO:25), or a variant thereof, such as variant having one individual amino acid substitution, and optionally comprising a fatty acid molecule; or such as a variant comprising a fatty acid molecule.

In a particular embodiment, the GIPR antagonist GIP peptide is selected from the group consisting of: EGTFISEYSIAibLEKIKQQEFVEWLLAQKPSSGAPPPS-C16-diacid/18K; SEQ ID NO:6; GIP(3-30)+Cex(31-39) [D9E;A13Aib;M14L;D15E;H18K;D21E;N24E], XGTFISEYSIAibNIeEKIKQQEFVEWLLAQKPSSGAPPPS-2xAEEAc+yGlu-C18-diacid/18K; SEQ ID NO:12; GIP(3-30)+Cex(31-39) [E3Glutaric acid(X);D9E;A13Aib; M14Nle;D15E;H18K;D21E;N24E], and XGTFISEYSIAibLEKIKQQEFVEWLLAQKPSSGAPPPS-C16-diacid/18K; SEQ ID NO:11; GIP(3-30)+Cex(31-39) [E3Glutaric acid(X);D9E;A13Aib;M14L;D15E;H18K;D21E;N24E], or a functional variant thereof comprising 1, 2 or 3 individual amino acid substitutions;

and wherein the GLP-1 receptor agonist is (Arg34)hGLP-1(7-37) (SEQ ID NO:25) comprising a fatty acid molecule attached to the lysine at position 26 of said (SEQ ID NO:25), or a variant thereof, such as a variant having one individual amino acid substitution. In one embodiment said variant has an amino acid substitution at position 8.

In a particular embodiment, the GIPR antagonist GIP peptide is selected from the group consisting of: EGTFISEYSIAibLEKIKQQEFVEWLLAQKPSSGAPPPS-C16-diacid/18K; SEQ ID NO:6; GIP(3-30)+Cex(31-39) [D9E;A13Aib;M14L;D15E;H18K;D21E;N24E], XGTFISEYSIAibNIeEKIKQQEFVEWLLAQKPSSGAPPPS-2xAEEAc+yGlu-C18-diacid/18K; SEQ ID NO:12; GIP(3-30)+Cex(31-39) [E3Glutaric acid(X);D9E;A13Aib; M14Nle;D15E;H18K;D21E;N24E], and XGTFISEYSIAibLEKIKQQEFVEWLLAQKPSSGAPPPS-C16-diacid/18K; SEQ ID NO:11; GIP(3-30)+Cex(31-39) [E3Glutaric acid(X);D9E;A13Aib;M14L;D15E;H18K;D21E;N24E], or a functional variant thereof comprising 1, 2 or 3 individual amino acid substitutions;

and wherein the GLP-1 receptor agonist is liraglutide or semaglutide.

In a particular embodiment, the GIPR antagonist GIP peptide is selected from the group consisting of: EGTFISEYSIAibLEKIKQQEFVEWLLAQKPSSGAPPPS-C16-diacid/18K; SEQ ID NO:6; GIP(3-30)+Cex(31-39) [D9E;A13Aib;M14L;D15E;H18K;D21E;N24E], XGTFISEYSIAibNIeEKIKQQEFVEWLLAQKPSSGAPPPS-2xAEEAc+yGlu-C18-diacid/18K; SEQ ID NO:12; GIP(3-30)+Cex(31-39) [E3Glutaric acid(X);D9E;A13Aib; M14Nle;D15E;H18K;D21E;N24E], and XGTFISEYSIAibLEKIKQQEFVEWLLAQKPSSGAPPPS-C16-diacid/18K; SEQ ID NO:11; GIP(3-30)+Cex(31-39) [E3Glutaric acid(X);D9E;A13Aib;M14L;D15E;H18K;D21E;N24E], or a functional variant thereof comprising 1, 2 or 3 individual amino acid substitutions;

and wherein the GLP-1 receptor agonist is semaglutide.

In a particular embodiment, the GIPR antagonist GIP peptide is EGTFISEYSIAibLEKIKQQEFVEWLLAQKPSSGAPPPS-C16-diacid/18K; SEQ ID NO:6; GIP(3-30)+Cex(31-39) [D9E;A13Aib;M14L;D15E;H18K;D21E;N24E] or a functional variant thereof comprising 1, 2 or 3 individual amino acid substitutions,

and wherein the GLP-1 receptor agonist is semaglutide.

In a particular embodiment, the GIPR antagonist GIP peptide is XGTFISEYSIAibNIeEKIKQQEFVEWLLAQKPSSGAPPPS-2xAEEAc+yGlu-C18-diacid/18K; SEQ ID NO:12; GIP(3-30)+Cex(31-39) [E3Glutaric acid(X);D9E;A13Aib; M14Nle;D15E;H18K;D21E;N24E] or a functional variant thereof comprising 1, 2 or 3 individual amino acid substitutions,

and wherein the GLP-1 receptor agonist is semaglutide.

In a particular embodiment, the GIPR antagonist GIP peptide is XGTFISEYSIAibLEKIKQQEFVEWLLAQKPSSGAPPPS-C16-diacid/18K; SEQ ID NO: 11; GIP(3-30)+Cex(31-39) [E3Glutaricacid(X);D9E;A13Aib;M14L;D15E;H18K;D21E;N24E] or a functional variant thereof comprising 1, 2 or 3 individual amino acid substitutions,

and wherein the GLP-1 receptor agonist is semaglutide.

Pharmaceutical Composition and Formulation Whilst it is possible for the bioactive agents of the present disclosure to be administered as the raw chemicals (e.g. peptides), it is sometimes preferred to present them in the form of a pharmaceutical formulation. Such a pharmaceutical formulation may be referred to as a pharmaceutical composition, pharmaceutically acceptable composition or pharmaceutically safe composition.

Accordingly, further provided is a pharmaceutical formulation, which comprises one or more bioactive agents of the present disclosure, or a pharmaceutically acceptable salt or ester thereof, and a pharmaceutically acceptable carrier, excipient and/or diluent. The pharmaceutical formulations may be prepared by conventional techniques, e.g. as described in Remington: The Science and Practice of Pharmacy 2005, Lippincott, Williams & Wilkins.

Pharmaceutically acceptable salts of the instant peptide compounds, where they can be prepared, are also intended to be covered by this disclosure. These salts will be ones which are acceptable in their application to a pharmaceutical use. By that it is meant that the salt will retain the biological activity of the parent compound and the salt will not have untoward or deleterious effects in its application and use in treating diseases.

Pharmaceutically acceptable salts are prepared in a standard manner. If the parent compound is a base it may for example be treated with an excess of an organic or inorganic acid in a suitable solvent. If the parent compound is an acid, it may for example be treated with an inorganic or organic base in a suitable solvent.

A pharmaceutical formulation or a pharmaceutical composition according to the present disclosure comprises a GIPR antagonist GIP peptide, a GLP-1 receptor agonist, or both a GIPR antagonist GIP peptide and a GLP-1 receptor agonist according to the present disclosure.

The present disclosure relates to combination therapy of a GIPR antagonist GIP peptide and a GLP-1 receptor agonist.

In some embodiments of the present disclosure the GIPR antagonist GIP peptide and the GLP-1 receptor agonist are present in the same pharmaceutical formulation.

In some embodiments of the present disclosure the GIPR antagonist GIP peptide and the GLP-1 receptor agonist are present in two different pharmaceutical formulations, which may administered simultaneously, sequentially or separately to an individual in need thereof.

### Administration and Dosage

According to the present disclosure, a GIP peptide, a GLP-1 receptor agonist, or a composition comprising a GIP peptide and/or a GLP-1 receptor agonist as defined herein is administered to an individual in need of treatment in pharmaceutically effective doses or a therapeutically effective amount. The dosage requirements will vary with the particular drug composition employed, the route of administration and the particular subject being treated, which depend on the severity and the sort of the disorder as well as on the weight and general state of the subject. It will also be recognized by one skilled in the art that the optimal quantity and spacing of individual dosages of a peptide compound will be determined by the nature and extent of the condition being treated, the form, route and site of administration, and the particular patient being treated, and that such optima can be determined by conventional techniques. It will also be appreciated by one of skill in the art that the optimal course of treatment, i.e., the number of doses of a compound given per day for a defined number of days, can be ascertained using conventional course of treatment determination tests.

In one embodiment each of the bioactive agents are administered at least once daily, such as once daily.

In one embodiment each of the bioactive agents are administered in intermittent intervals, or intervals, whereby a dose is not administered every day.

In one embodiment one or more doses are administered once every second day, every third day, every fourth day, every fifth day, every sixth day, every week, every second week, every third week, every fourth week, every month, every fifth week, every sixth week, or intervals within those ranges (such as every 2 to 4 weeks, or 4 to 6 weeks).

In one embodiment, a dose is administered once every week, such as once weekly, such as in one dose per week.

In one embodiment, a dose of the GIPR antagonist GIP peptide of the present disclosure is administered at least once daily, such as once daily, such as once each second day, such as once each third day, such as once each fourth day, such as once each fifth day, such as once each sixth day, such as once weekly, such as once each second week (biweekly), such as once monthly.

In one embodiment, a dose of the GIPR antagonist GIP peptide of the present disclosure is administered once daily.

In one embodiment, a dose of the GIPR antagonist GIP peptide of the present disclosure is administered once weekly.

In one embodiment, a dose of the GIPR antagonist GIP peptide of the present disclosure is administered once each second week.

In one embodiment, a dose of the GIPR antagonist GIP peptide of the present disclosure is administered once monthly.

In one embodiment, a dose of the GLP-1 receptor agonist of the present disclosure is administered continuously, such as at least once daily, such as once daily, such as once each second day, such as once each third day, such as once each fourth day, such as once each fifth day, such as once each sixth day, such as once weekly, such as once each second week, such as once monthly.

In one embodiment, a dose of the GLP-1 receptor agonist of the present disclosure is administered once daily.

In one embodiment, a dose of the GLP-1 receptor agonist of the present disclosure is administered once weekly.

In one embodiment, a dose of the GLP-1 receptor agonist of the present disclosure is administered once each second week (biweekly dosage).

In one embodiment, the GIPR antagonist GIP peptide according to the present disclosure is administered at a dosage of about 15 nmol/kg, such as about 20 nmol/kg, such as about 25 nmol/kg, such as about 30 nmol/kg, such as about 35 nmol/kg, such as about 40 nmol/kg, such as about 45 nmol/kg, such as about 50 nmol/kg, such as about 55 nmol/kg, such as about 60 nmol/kg, such as about 65 nmol/kg, such as about 70 nmol/kg, such as about 75 nmol/kg, such as about 80 nmol/kg, such as about 85 nmol/kg, such as about 90 nmol/kg, such as about 95 nmol/kg, such as about 100 nmol/kg, such as about 110 nmol/kg, such as about 120 nmol/kg, such as about 125 nmol/kg, such as about 150 nmol/kg, such as about 175 nmol/kg, such as about 200 nmol/kg, such as about 250 nmol/kg, such as about 300 nmol/kg, such as about 350 nmol/kg, such as about 400 nmol/kg, such as about 450 nmol/kg, such as about 500 nmol/kg, such as about 550 nmol/kg, such as about 600 nmol/kg, such as about 650 nmol/kg, such as about 700 nmol/kg, such as about 750 nmol/kg, such as about 800 nmol/kg, such as about 850 nmol/kg, such as about 900 nmol/kg, such as about 950 nmol/kg, such as about 1000 nmol/kg, such as about 1050 nmol/kg, such as about 1100 nmol/kg, such as about 1150 nmol/kg, such as about 1200 nmol/kg, such as about 1250 nmol/kg, such as about 1300 nmol/kg, such as about 1350 nmol/kg, such as about 1400 nmol/kg, such as about 1450 nmol/kg, such as about 1500 nmol/kg, such as about 2000 nmol/kg, such as about 2500 nmol/kg. In one embodiment, said dose of the GIPR antagonist GIP peptide according to the present disclosure is administered at a daily dosage, at a weekly dosage, at a biweekly dosage or at a monthly dosage.

In one embodiment, the GIPR antagonist GIP peptide according to the present disclosure is administered at a once weekly dosage of about 15 nmol/kg, such as about 20 nmol/kg, such as about 25 nmol/kg, such as about 30 nmol/kg, such as about 35 nmol/kg, such as about 40 nmol/kg, such as about 45 nmol/kg, such as about 50 nmol/kg, such as about 55 nmol/kg, such as about 60 nmol/kg, such as about 65 nmol/kg, such as about 70 nmol/kg, such as about 75 nmol/kg, such as about 80 nmol/kg, such as about 85 nmol/kg, such as about 90 nmol/kg, such as about 95 nmol/kg, such as about 100 nmol/kg, such as about 110 nmol/kg, such as about 120 nmol/kg, such as about 125 nmol/kg, such as about 150 nmol/kg, such as about 175 nmol/kg, such as about 200 nmol/kg, such as about 250 nmol/kg, such as about 300 nmol/kg, such as about 350 nmol/kg, such as about 400 nmol/kg, such as about 450 nmol/kg, such as about 500 nmol/kg, such as about 550 nmol/kg, such as about 600 nmol/kg, such as about 650 nmol/kg, such as about 700 nmol/kg, such as about 750 nmol/kg, such as about 800 nmol/kg, such as about 850 nmol/kg, such as about 900 nmol/kg, such as about 950 nmol/kg, such as about 1000 nmol/kg, such as about 1050 nmol/kg, such as about 1100 nmol/kg, such as about 1150 nmol/kg, such as about 1200 nmol/kg, such as about 1250 nmol/kg, such as about 1300 nmol/kg, such as about 1350 nmol/kg, such as about 1400 nmol/kg, such as about 1450 nmol/kg, such as about 1500 nmol/kg, such as about 2000 nmol/kg, such as about 2500 nmol/kg.

In one embodiment, the GIPR antagonist GIP peptide according to the present disclosure is administered at a dosage of 15 to 20 nmol/kg, such as 20 to 25 nmol/kg, such as 25 to 30 nmol/kg, such as 30 to 35 nmol/kg, such as 35 to 40 nmol/kg, such as 40 to 45 nmol/kg, such as 45 to 50 nmol/kg, such as 50 to 60 nmol/kg, such as 60 to 70 nmol/kg, such as 70 to 80 nmol/kg, such as 80 to 90 nmol/kg, such as 90 to 100 nmol/kg, such as 100 to 125 nmol/kg, such as 125 to 150 nmol/kg, such as 150 to 175 nmol/kg, such as 175 to 200 nmol/kg, such as 200 to 250 nmol/kg, such as 250 to 300 nmol/kg, such as 300 to 350 nmol/kg, such as 350 to 400 nmol/kg, such as 400 to 450 nmol/kg, such as 450 to 500 nmol/kg, such as 500 to 550 nmol/kg, such as 550 to 600 nmol/kg, such as 600 to 650 nmol/kg, such as 650 to 700 nmol/kg, such as 700 to 750 nmol/kg, such as 750 to 800 nmol/kg, such as 800 to 850 nmol/kg, such as 850 to 900 nmol/kg, such as 900 to 1000 nmol/kg, such as 1000 to 1050 nmol/kg, such as 1050 to 1100 nmol/kg, such as 1100 to 1200 nmol/kg, such as 1200 to 1250 nmol/kg, such as 1250 to 1300 nmol/kg, such as 1300 to 1350 nmol/kg, such as 1350 to 1400 nmol/kg, such as 1400 to 1450 nmol/kg, such as 1450 to 1500 nmol/kg, such as 1500 to 1750 nmol/kg, such as 1750 to 2000 nmol/kg, such as 2000 to 2500 nmol/kg. In one embodiment, said dose of the GIPR antagonist GIP peptide according to the present disclosure is administered at a daily dosage, at a weekly dosage, at a biweekly dosage or at a monthly dosage.

In one embodiment, the GLP-1 receptor agonist of the present disclosure is administered at a dosage of about 5 μg/kg, such as about 10 μg/kg, such as about 15 μg/kg, such as about 20 μg/kg, such as about 25 μg/kg, such as about 30 μg/kg, such as about 40 μg/kg, such as about 50 μg/kg, such as about 75 μg/kg, such as about 100 μg/kg, such as about 200 μg/kg, such as about 300 μg/kg, such as about 400 μg/kg, such as about 500 μg/kg, such as about 600 μg/kg, such as about 700 μg/kg, such as about 800 μg/kg, such as about 900 μg/kg, such as about 1 mg/kg, such as about 5 mg/kg, such as about 10 mg/kg, such as about 25 mg/kg, such as about 50 mg/kg, such as about 100 mg/kg. In one embodiment, said dose of the GLP-1 receptor agonist according to the present disclosure is administered at a daily dosage, at a weekly dosage, at a biweekly dosage or at a monthly dosage.

In one embodiment, the GLP-1 receptor agonist of the present disclosure is administered at a dosage of 5 to 10 μg/kg, such as 10 to 15 μg/kg, such as 15 to 20 μg/kg, such as 20 to 25 μg/kg, such as 25 to 30 μg/kg, such as 30 to 40 μg/kg, such as 40 to 50 μg/kg, such as 50 to 75 μg/kg, such as 75 to 100 μg/kg, such as 100 to 200 μg/kg, such as 200 to 300 μg/kg, such as 300 to 400 μg/kg, such as 400 to 500 μg/kg, such as 500 to 600 μg/kg, such as 600 to 700 μg/kg, such as 700 to 800 μg/kg, such as 800 to 900 μg/kg, such as 900 to 1 mg/kg, such as 1 to 5 mg/kg, such as 5 to 10 mg/kg, such as 10 to 25 mg/kg, such as 25 to 50 mg/kg, such as 50 to 100 mg/kg,

In one embodiment, said dose of the GLP-1 receptor agonist according to the present disclosure is administered at a daily dosage, at a weekly dosage, at a biweekly dosage or at a monthly dosage.

In one embodiment, a dose of the GLP-1 receptor agonist is administered at a daily dosage of about 300 μg/kg, such as about 400 μg/kg.

In one embodiment, a dose of the GLP-1 receptor agonist is administered separately from a dose of the GIP peptide. For example, a dose of the GLP-1 receptor agonist may be administered on the same day, but at a different point in time of the dose of the GIP peptide. For example, a dose of the GLP-1 receptor agonist may be administered on a different day than a dose of the GIP peptide.

In one embodiment, a dose of the GLP-1 receptor agonist is administered separately from a dose of the GIP peptide, such that the GLP-1 receptor agonist and the GIP peptide are contained in separate compositions.

In one embodiment, a dose of the GLP-1 receptor agonist is administered simultaneously with a dose of the GIP peptide. Simultaneously encompass at essentially the same time and exactly at the same time. Simultaneous administration encompass wherein the two components are comprised in the same composition.

In one embodiment, a dose of the GLP-1 receptor agonist and the GIP peptide are administered sequentially, such as administering the GLP-1R agonist prior to or after administration of a dose of the GIP peptide.

### Routes of Administration

It will be appreciated that the preferred route of administration will depend on the general condition and age of the subject to be treated, the nature of the condition to be treated, the location of the tissue to be treated in the body and the active ingredient chosen, but may for example be subcutaneous.

### Systemic Treatment

For systemic treatment according to the present disclosure the route of administration is capable of introducing the bioactive agents into the blood stream to ultimately target the sites of desired action.

Such routes of administration are any suitable routes, such as an enteral route (including the oral, rectal, nasal, pulmonary, buccal, sublingual, transdermal, intracisternal and intraperitoneal administration), and/or a parenteral route (including subcutaneous, intramuscular, intrathecal, intracerebral, intravenous and intradermal administration).

### Parenteral Administration

Parenteral administration is any administration route not being the oral/enteral route whereby the medicament avoids first-pass degradation in the liver. Accordingly, parenteral administration includes any injections and infusions, for example bolus injection or continuous infusion, such as intravenous administration, intramuscular administration or subcutaneous administration. Furthermore, parenteral administration includes inhalations and topical administration.

Accordingly, the bioactive agents may be administered topically to cross any mucosal membrane of an animal to which the biologically active substance is to be given, e.g. in the nose, vagina, eye, mouth, genital tract, lungs, gastrointestinal tract, or rectum, preferably the mucosa of the nose, or mouth, and accordingly, parenteral administration may also include buccal, sublingual, nasal, rectal, vaginal and intraperitoneal administration as well as pulmonal and bronchial administration by inhalation or installation. Also, the agents may be administered topically to cross the skin.

According to an advantageous embodiment of the invention, the GIP peptide and/or the GLP-1 receptor agonist are administered subcutaneously.

In some embodiments of the present disclosure, the GIP peptide and/or the GLP-1 receptor agonist are administered systemically.

In some embodiments of the present disclosure, the GIP peptide and/or the GLP-1 receptor agonists are administered by infusion.

In some embodiments of the present disclosure, the GIP peptide and/or the GLP-1 receptor agonists are administered by injection.

In some embodiments of the present disclosure, the GIP peptide and/or the GLP-1 receptor agonists are administered parenterally, including subcutaneous, intramuscular, intrathecal, intracerebral, intravenous and intradermal administration.

### Local Treatment

The bioactive agent according to the invention may in one embodiment be used as a local treatment, i.e. be introduced directly to the site(s) of action. Accordingly, the bioactive agent may be applied to the skin or mucosa directly, or the bioactive agent may be injected into the site of action, for example into the diseased tissue or to an end artery leading directly to the diseased tissue. These administration forms preferably avoid the blood brain barrier.

### EXAMPLES

### Example 1. In Vivo Study

### Tested Peptides

### GIPR Antagonist GIP Peptides:

Compound 1: EGTFISEYSIAibLEKIKQQEFVEWLLAQKPSSGAPPPS-C16-diacid/18K (GIP(3-30)+Cex(31-39) [D9E;A13Aib;M14L;D15E;H18K;D21E;N24E]), which corresponds to SEQ ID NO:6 with a C16-diacid attached at the side chain of the Lysine at position 16 (18K if the numbering of native human GIP is considered).

Compound 2: XGTFISEYSIAibNIeEKIKQQEFVEWLLAQKPSSGAPPPS-2xAEEAc+yGlu-C18-diacid/18K (GIP(3-30)+Cex(31-39) [E3Glutaric acid(X);D9E;A13Aib;M14Nle;D15E;H18K;D21E; N24E], which corresponds to SEQ ID NO:12 with a C18-diacid attached at the side chain of the Lysine at position 16 (18K if the numbering of native human GIP is considered) via the linker 2xAEEAc+yGlu.

### Glp-1 Agonist: Liraglutide.

### Materials and Methods

The cynomolgus monkey efficacy study was performed by Kunming Biomed International (KBI) in China. The study was approved by KBI's Institutional Animal Care and Use Committee. High fat diet (HFD) induced obese cynomolgus monkeys (Macaca fascicularis, male, >7.5 kg, fasting glucose<90 mg/dL, fasting insulin<60 μU/mL, >8 years of age) were obtained from KBI's stock colony. Lighting in animal holding rooms was maintained on 12:12 hour light:dark cycle, and the ambient temperature and humidity range was set at 18-29 degrees C. and 30 to 90%, respectively. The monkeys were individually housed in cages arranged to allow visual and auditory contacts with conspecifics. The monkeys were provided with cage toys for environmental enrichment to ensure adequate welfare and psychological well-being.

8 days prior to the treatment phase, and KBI's proprietary HFD was provided adlibitum which is in contrast to the defined amount of 100 g that the monkeys were normally served. During the entire treatment phase (42 days) and washout period (14 days) monkeys were provided three meals per day consisting of 50 g of KBI proprietary standard monkey formula feed in the morning 9:00-10:00 AM, one regular apple (150 g) in the afternoon 14:00-15:00 PM, and ad libitum of KBI proprietary HFD in the evening 16:00-17:00 PM. Water was provided adlibitum. All food was withdrawn at 17:00 PM, hence, the monkeys were always fasted overnight.

The cynomolgus monkeys were acclimated/trained to experimental procedures for three weeks followed by one week with continued training and introduction of adlibitum HFD prior to start of treatment. The monkeys were randomly sorted into six separate dose groups with 10 animals each based on acclimation data sets.

During the treatment period all monkeys received daily SC injections after breakfast with the respective test substance for a total of 42 days. Liraglutide was given in dose escalation fashion over seven days to circumvent nausea and vomiting issues associated with GLP-1R agonism. The final dosing of liraglutide was 0.03 mg/kg. Compound 1 dosing was 540 nmol/kg and due to the long half live of Compound 2, it was dosed at 1440 nmol/kg for the first three study days to reach a steady state plasma concentration simultaneously with Compound 1, the dosing was 540 nmol/kg for the remaining 39 study days. The same concentrations were used for the combination treatment groups with Compound 1+liraglutide and Compound 2+liraglutide, respectively. All treatment groups and the vehicle group received injection volumes based on individual body weights. After the 42 days of treatment, body weight, food intake, and blood/serum chemistries were monitored for an additional 14-day washout period. Blood collections and dosing occurred in conscious monkeys for the entire study.

Daily food intake measurements were recorded during the entire duration of the study from the acclimation period until the end of the washout period. After each feeding time, all of the remaining food was withdrawn and intake was determined by weighing the left-over food.

Body weights were measured every third to fourth day during the study period. The monkeys were non-sedated and were transferred into small transfer cages and transported to a weighing scale. The tare weights of the transfer cages were determined prior to weighing the monkeys inside the transfer cages. The monkeys were weighed twice inside the transfer cages and recordings were made to the nearest 0.1 kg.

Whole blood sample was collected from a peripheral vein after an overnight fast according to the specified time points and transferred directly into serum separation tubes (SST). Serum was separated by centrifugation at 2500×g for 10 min at 4° C. Insulin, CTX and PTH levels were analyzed using the Cobas e411 Immunology analyzer. Other parameters were analyzed using the Roche C311 or C501 biochemical analyzer.

### Results

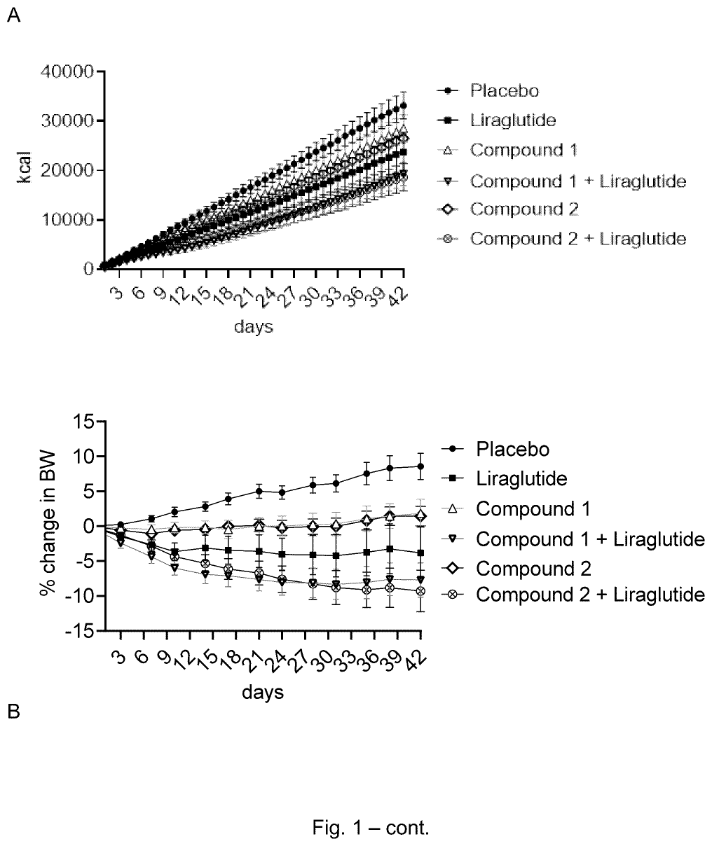

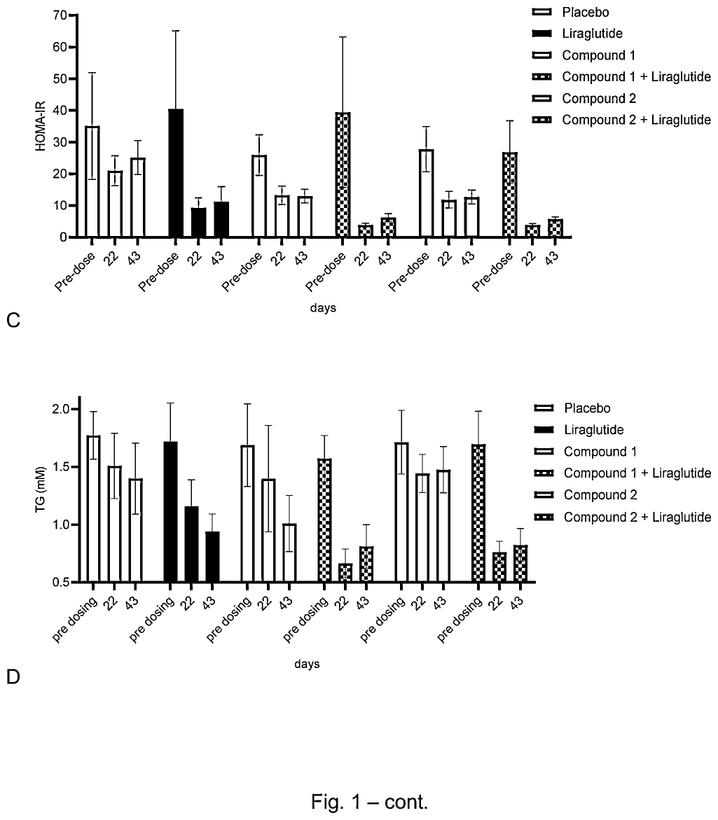

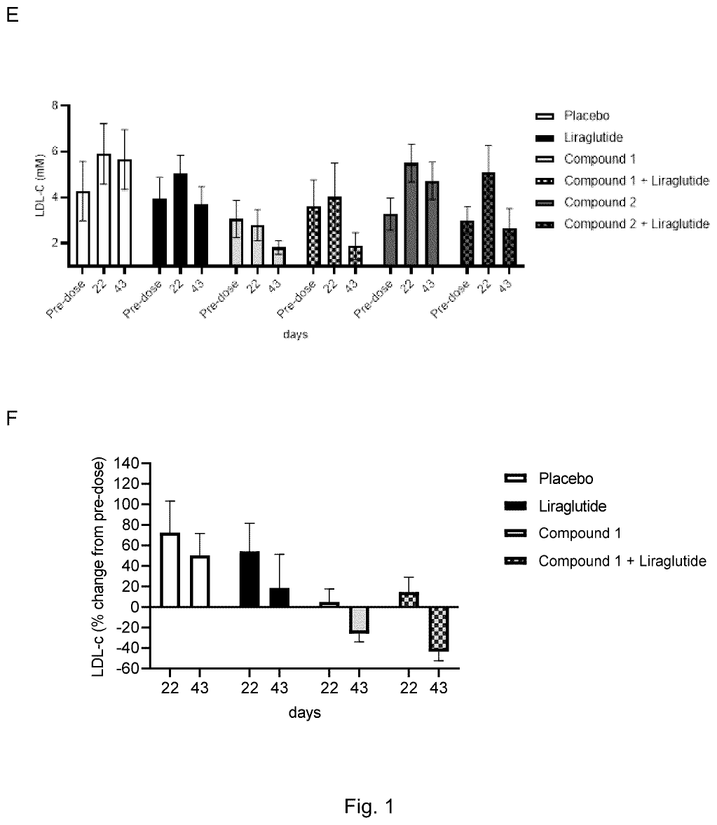

All treatment groups had a lower cumulative energy intake compared to placebo. Compound 1+liraglutide and Compound 2+liraglutide showed the most prominent drop in energy intake of all groups, see FIG. 1A. From this, it can be concluded that combining our GIP antagonist with the GLP-1 receptor agonist liraglutide have at least additive effects on total energy intake.

The results on cumulative energy intake agree with the results on body weight loss. The placebo group had a total average weight gain of 9% on the last treatment day (day 42) whereas mono-treatments with Compound 1 or Compound 2 maintained body weight during the study period, demonstrating clear efficacy of both mono-treatments. Treatment with liraglutide resulted in a total average weight loss of 4% and treatment with Compound 1+liraglutide and Compound 2+liraglutide resulted in average total weight losses of 8% and 9% (17% and 18% vs placebo), respectively. These results clearly show that combining our GIP antagonists with the GLP-1 receptor agonist liraglutide is more efficacious on body weight loss than treatment with liraglutide alone.

Weight-loss is well known to cause improvements in insulin sensitivity and lipid profiles, and it is therefore often difficult to assess whether a drug directly affects these parameters in the presence of weight-loss. In this study, the obese NHPs treated with monotherapy of Compound 1 or Compound 2 were protected against the weight-gain observed in the placebo-treated animals and importantly, resulted in no change in weight throughout the study period. This is important for the interpretation of the following data.

Homeostatic Model Assessment for Insulin Resistance (HOMA-IR) was calculated and indicates improvements to insulin sensitivity for the treatment groups with the highest insulin sensitivity observed in the combination groups (at day 43 the average HOMA-IR was 25.1, 11.2, 13.0, 6.2, 12.7, and 5.8 for placebo, liraglutide, Compound 1, Compound 1+liraglutide, Compound 2, and Compound 2+liraglutide, respectively), see FIG. 1C. Importantly, as both Compound 1 and Compound 2 were weight-stable throughout the study period, the effects on insulin sensitivity were independent of weight-loss.

Animals treated with Compound 1 either alone or in combination with liraglutide had reduced fasting triglycerides (see FIG. 1D). Both combination groups reached a significant reduction after 22 days of treatment, which indicates at least additivity between GIPR antagonism and GLP-1 agonism in lowering fasting triglycerides. Importantly, in both Compound 1 treated groups, there was a trend for lowering LDL-c which was not seen in any other treatment groups (FIGS. 1E and F). Calculating the individual %-change in LDL-c from predose, indicates that the placebo-group and the GLP-1 group had increased LDL levels during the study period while a 26% and 43% reduction in LDL-c was reached for the Compound 1− and Compound 1+liraglutide-group, respectively. Intriguingly, as Compound 1's effects on triglycerides and LDL-c are also observed in the monotreated group, these improvements are weight-loss independent.

During the study a large battery of safety biomarkers were assessed including markers of bone metabolism, liver function, and hematology and no changes out of the normal range were detected.

Sequence overview

EGTFISEYSAibANleEKIKQQDFVEWLLAQKPSSGAPPPS; SEQ ID NO: 1; GIP(3-30)
[D9E; I12Aib; M14Nle; D15E; H18K; N24E],
EGTFISEYSIAibMEKIKQQDFVEWLLAQKPSSGAPPPS; SEQ ID NO: 2; GIP(3-30)
[D9E; A13Aib; D15E; H18K; N24E],
EGTFISDYSIAibMDKIKQQDFVEWLLAQKPSSGAPPPS; SEQ ID NO: 3; GIP(3-30)
[A13Aib; H18K; N24E],
EGTFISDYSIAibLDKIKQQDFVEWLLAQKPSSGAPPPS; SEQ ID NO: 4; GIP(3-30)
[A13Aib; M14L; H18K; N24E],
EGTFISDYSIAibNleDKIKQQDFVEWLLAQKPSSGAPPPS; SEQ ID NO: 5; GIP(3-30)
[A13Aib; M14Nle; H18K; N24E],
EGTFISEYSIAibLEKIKQQEFVEWLLAQKPSSGAPPPS; SEQ ID NO: 6; GIP(3-30)
[D9E; A13Aib; M14L; D15E; H18K; D21E; N24E],
EGTFISEYSIAibNleEKIKQQEFVEWLLAQKPSSGAPPPS; SEQ ID NO: 7; GIP(3-30)
[D9E; A13Aib; M14Nle; D15E; H18K; D21E; N24E],
EGTFISEYSIALEKIKQQEFVEWLLAQKPSSGAPPPS; SEQ ID NO: 8; GIP(3-30)
[D9E; M14L; D15E; H18K; D21E; N24E],
EGTFISEYSIANleEKIKQQEFVEWLLAQKPSSGAPPPS; SEQ ID NO: 9; GIP(3-30)
[D9E; M14Nle; D15E; H18K; D21E; N24E],
EGTFISEYSIAibLEKIKQQDFVEWLLAQKPSSGAPPPS; SEQ ID NO: 10; GIP(3-30)
[D9E; A13Aib; M14L; D15E; H18K; N24E],
XGTFISEYSIAibLEKIKQQEFVEWLLAQKPSSGAPPPS; SEQ ID NO: 11; GIP(3-30)
[E3Glutaric acid(X); D9E; A13Aib; M14L; D15E; H18K; D21E; N24E],
XGTFISEYSIAibNleEKIKQQEFVEWLLAQKPSSGAPPPS; SEQ ID NO: 12; GIP(3-30)
[E3Glutaric acid(X); D9E; A13Aib; M14Nle; D15E; H18K; D21E; N24E],
XGTFISEYSIALEKIKQQEFVEWLLAQKPSSGAPPPS; SEQ ID NO: 13; GIP(3-30)
[E3Glutaric acid(X); D9E; M14L; D15E; H18K; D21E; N24E],
XGTFISEYSIAibMEKIKQQEFVEWLLAQKPSSGAPPPS; SEQ ID NO: 14; GIP(3-30)
[E3Glutaric acid(X); D9E; A13Aib; D15E; H18K; D21E; N24E],
EGTFISEYSIAibMEKIKQQEFVEWLLAQKPSSGAPPPS; SEQ ID NO: 15; GIP(3-
30)[D9E; A13Aib; D15E; H18K; D21E; N24E],
EGTFISEYSIAMEKIKQQEFVEWLLAQKPSSGAPPPS; SEQ ID NO: 16; GIP(3-30)
[D9E; D15E; H18K; D21E; N24E],
EGTFISEYSIAibLDKIKQQDFVEWLLAQKPSSGAPPPS; SEQ ID NO: 17; GIP(3-30)
[D9E; A13Aib; M14L; H18K; N24E],
XGTFISDYSIAibMDKIKQQDFVEWLLAQKPSSGAPPPS; SEQ ID NO: 18; GIP(3-30)
[E3Glutaric acid(X); A13Aib; H18K; N24E],
HAibEGTFTSDVSSYLEGQAAKEFIAWLVRGRG; SEQ ID NO: 19; semaglutide,
XGTFISEYSIAibLEKIKQQEFVEWLLAQKPSSGAPPPS; SEQ ID NO: 20; GIP(3-30)
[E3Succinic acid(X); D9E; A13Aib; M14L; D15E; H18K; D21E; N24E],
XGTFISEYSIAibLEKIKQQEFVEWLLAQKPSSGAPPPS; SEQ ID NO: 21; GIP(3-30)
[E3Adipic acid(X); D9E; A13Aib; M14L; D15E; H18K; D21E; N24E],
HGEGTFTSDLSKQMEEEAVRLFIEWLKNGGPSSGAPPPS; SEQ ID NO: 22;
exenatide,
HGEGTFTSDLSKQMEEEAVRLFIEWLKNGGPSSGAPPSKKKKKK; SEQ ID NO: 23;
lixisenatide,
HGEGX₁FX₂SDVSSYLEGQAAKEFX₃DAWLVKGR; SEQ ID NO: 24; albiglutide [X₁ is
allo-threonine; X₂ is allo-threonine; X₃ is alle (allo-isoleucine)],
HAEGTFTSDVSSYLEGQAAKEFIAWLVKGRG; SEQ ID NO: 25; liraglutide;
HX₁EGTFTSDVSSYLEGQAAKEFIAWLVKX₂R; SEQ ID NO: 26; taspoglutide [X₁ is Aib;
X₂ is Aib],
HGEGTFTSDVSSYLEEQAAKEFIAWLVKGGGGGGGSGGGGGSGGGGSAESKYGPPC
PPCPAPEAAGGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNYKTTPPVL
DSDGSFFLYSRLTVDKSRWQEGNVFSCSVMHEALHNHYTQKSLSLSLG; SEQ ID
NO: 27; dulaglutide,
HAEATFTSDVSSYLEGQAAKEFIAWLVKGR; SEQ ID NO: 28; A10-hGLP-1(7-36),
HGEGTFTSDLSKQMEEEAVRLFIEWLKNGG; SEQ ID NO: 29; Ex4(1-30),
DLSKQMEEEAVRLFIEWLKNGGPSSGAPPPS; SEQ ID NO: 30; Ex4(9-39),
DLSKQMEEEAVRLFIEWLKNGG; SEQ ID NO: 31; Ex4(9-30),
HDEFERHAEGTFTSDVSSYLEGQAAKEFIAWLVKGRG; SEQ ID NO: 32; hGLP-1
(same as hGLP-1(1-37)),
HAEGTFTSDVSSYLEGQAAKEFIAWLVKGR; SEQ ID NO: 33; hGLP-1(7-36),
HAEGTFTSDVSSYLEGQAAKEFIAWLVKGRG; SEQ ID NO: 34; hGLP-1(7-37),
HDEFERHAEGTFTSDVSSYLEGQAAKEFIAWLVKGR; SEQ ID NO: 35; hGLP-1(1-36),
EGTFTSDVSSYLEGQAAKEFIAWLVKGR; SEQ ID NO: 36; hGLP-1(9-36),
AAEGTFTSDVSSYLEGQAAKEFIAWLVKGR; SEQ ID NO: 37; A7-hGLP-1(7-36),
EGTFISEYSIAMEKIKQQDFVEWLLAQKPSSGAPPPS; SEQ ID NO: 38; GIP(3-
30) + Cex(31-39)[D9E; D15E; H18K; N24E].

1. A glucose-dependent insulinotropic peptide receptor (GIPR) antagonist GIP peptide consisting of amino acid sequence SEQ ID NO:1:
(SEQ ID NO: 1)
3 - 4 - 5 - 6 7 8 9 10 11 12 13
X₁ - G - T - F - I - S - E - Y - S - I - Aib -
14 15 16 17 18 19 20 21 22 23 24 25
X₂ - E - K - I - K - Q - Q - E - F - V - E - W -
26 27 28 29 30 31 32 33 34 35 36 37
L - L - A - Q - K - P - S - S - G - A - P - P -
38 39
P - S,
wherein X₁ is E, glutaric acid, adipic acid or succinic acid;
wherein X₂ is M, L or Nle;
or a functional variant thereof, wherein the amino acid sequence of said variant differs from SEQ ID NO:1 in that the amino acid sequence of the variant comprises 1, 2 or 3 individual amino acid substitutions,
wherein said peptide comprises a fatty acid molecule attached to the side chain amino group of the amino acid residue at position 18 of SEQ ID NO:1, or said functional variant thereof,
for use in a method of treating obesity, obesity-related disorders and/or dyslipidemia, said method comprising one or more steps of co-administering a glucagon-like peptide-1 (GLP-1) receptor agonist.

2. A glucose-dependent insulinotropic peptide receptor (GIPR) antagonist GIP peptide consisting of amino acid sequence SEQ ID NO:1:
(SEQ ID NO: 1)
3 - 4 - 5 - 6 7 8 9 10 11 12 13
X₁ - G - T - F - I - S - E - Y - S - I - Aib -
14 15 16 17 18 19 20 21 22 23 24 25
X₂ - E - K - I - K - Q - Q - E - F - V - E - W -
26 27 28 29 30 31 32 33 34 35 36 37
L - L - A - Q - K - P - S - S - G - A - P - P -
38 39
P - S,
wherein X₁ is E, glutaric acid, adipic acid or succinic acid;
wherein X₂ is M, L or Nle;
or a functional variant thereof, wherein the amino acid sequence of said variant differs from SEQ ID NO:1 in that the amino acid sequence of the variant comprises 1, 2 or 3 individual amino acid substitutions,
wherein said peptide comprises a fatty acid molecule attached to the side chain amino group of the amino acid residue at position 18 of SEQ ID NO:1, or said functional variant thereof,
for use in a method of inhibiting or reducing one or more of i) food intake, ii) body weight, iii) fasting blood levels of glucose, iv) fasting blood levels of insulin, v) insulin resistance, vi) fasting blood levels of triglycerides, vii) fasting blood levels of cholesterol and viii) fasting blood levels of low-density lipoprotein (LDL) cholesterol.

3. The peptide for use according to any one of the preceding claims, wherein said GLP-1 receptor agonist is a GLP-1 peptide.

4. The peptide for use according to any one of the preceding claims, wherein said GLP-1 peptide is a protease-resistant analogue of hGLP-1.

5. The peptide for use according to any one of the preceding claims, wherein said GLP-1 peptide is selected from the group consisting of exenatide (SEQ ID NO:22), Lixisenatide (SEQ ID NO:23), albiglutide (SEQ ID NO:24), liraglutide (SEQ ID NO:25), taspoglutide (SEQ ID NO:26), dulaglutide (SEQ ID NO:27), semaglutide (SEQ ID NO:19), efpeglenatide, exendin-4 (Ex4; SEQ ID NO:22), Ex4(1-30) (SEQ ID NO:29), Ex4(9-39) (SEQ ID NO:30), Ex(9-30) (SEQ ID NO:28), hGLP-1(1-37) (SEQ ID NO:32), hGLP-1(7-36) (SEQ ID NO:33), hGLP-1(7-37) (SEQ ID NO:34), hGLP-1(1-36) (SEQ ID NO:35), hGLP-1(9-36) (SEQ ID NO:36), A7-hGLP-1(7-36) (SEQ ID NO:37) and A10-hGLP-1(7-36) (SEQ ID NO:28), or a functional variant thereof.

6. The peptide for use according to any one of the preceding claims, wherein said GLP-1 peptide is hGLP-1(7-37), or a variant thereof, such as a variant having one or two individual amino acid substitutions, and optionally comprising a fatty acid molecule.

7. The peptide for use according to any one of the preceding claims, wherein said GLP-1 peptide is (Arg34)hGLP-1(7-37) (SEQ ID NO:25), or a variant thereof, such as variant having one amino acid substitution, and optionally comprising a fatty acid molecule.

8. The peptide for use according to any one of the preceding claims, wherein the GLP-1 receptor agonist is (Arg34)hGLP-1(7-37) (SEQ ID NO:25) comprising a fatty acid molecule attached to the lysine at position 26 of said (SEQ ID NO:25), or a variant thereof, such as a variant having one amino acid substitution.

9. The peptide for use according to any one of the preceding claims, wherein said GLP-1 peptide is selected from (Arg34)hGLP-1(7-37) (SEQ ID NO:25) or (Aib8)(Arg34)hGLP-1(7-37) (SEQ ID NO:19), comprising a fatty acid molecule attached to the lysine at position 26.

10. The peptide for use according to any one of the preceding claims, wherein said GLP-1 peptide is liraglutide or semaglutide.

11. The peptide for use according to any one of the preceding claims, wherein said GLP-1 peptide is semaglutide.

12. The peptide for use according to any one of the preceding claims, wherein said GLP-1 peptide is a liquid formulation of semaglutide.

13. The peptide for use according to any one of the preceding claims, wherein said GLP-1 peptide is a solid oral dosage form composition of semaglutide.

14. The peptide for use according to any one of the preceding claims, wherein said GLP-1 receptor agonist is a non-peptide GLP-1 receptor agonist.

15. The peptide for use according to any one of the preceding claims, wherein said non-peptide GLP-1 receptor agonist is a small molecule GLP-1 receptor agonist.

16. The peptide for use according to any one of the preceding claims, wherein said small molecule GLP-1 receptor agonist is selected from the group consisting of Boc5, WB4-24, OWL833, TT-OAD2, LY3502970, PF 06882961.

17. The peptide for use according to any one of the preceding claims, wherein said non-peptide GLP-1 receptor agonist is a GLP-1 receptor activating antibody.

18. The peptide for use according to any one of the preceding claims, wherein said GLP-1 receptor agonist is a dual or triple acting GLP-1 receptor agonist, such as a GLP-1/glucagon dual agonist.

19. The peptide for use according to any one of the preceding claims, wherein X₁ of SEQ ID NO:1 is E or glutaric acid.

20. The peptide for use according to any one of the preceding claims, wherein X₂ of SEQ ID NO:1 is L or Ne.

21. The peptide for use according to any one of the preceding claims, wherein X₁ of SEQ ID NO:1 is E and X₂ of SEQ ID NO:1 is L.

22. The peptide for use according to any one of the preceding claims, wherein X₁ of SEQ ID NO:1 is glutaric acid and X₂ of SEQ ID NO:1 is L.

23. The peptide for use according to any one of the preceding claims, wherein X₁ of SEQ ID NO:1 is glutaric acid and X₂ of SEQ ID NO:1 is Nle.

24. The peptide for use according to any one of the preceding claims, wherein the amino acid sequence of said variant differs from SEQ ID NO:1 in that the amino acid sequence of the variant comprises 3 individual amino acid substitutions, such as 2 individual amino acid substitutions, such as 1 amino acid substitution.

25. The peptide for use according to any one of the preceding claims, wherein said 1, 2 or 3 individual amino acid substitutions are conservative amino acid substitutions.

26. The peptide for use according to any one of the preceding claims, wherein the amino acid sequence of said variant differs from SEQ ID NO:1 in that the amino acid sequence of the variant comprises 1, 2 or 3 individual amino acid substitutions at any one of positions 4 to 8, 10 to 13, 16, 17, 19, 20, 22, 23, 25 to 39 of SEQ ID NO:1.

27. The peptide for use according to any one of the preceding claims, wherein the GIPR antagonist GIP peptide is selected from the group consisting of:
SEQ ID NO: 1
EGTFISEYSAibANleEKIKQQDFVEWLLAQKPSSGAPPPS;
GIP(3-30)[D9E; I12Aib; M14Nle; D15E; H18K; N24E],
SEQ ID NO: 2
EGTFISEYSIAibMEKIKQQDFVEWLLAQKPSSGAPPPS;
GIP(3-30)[D9E; A13Aib; D15E; H18K; N24E],
SEQ ID NO: 3
EGTFISDYSIAibMDKIKQQDFVEWLLAQKPSSGAPPPS;
GIP(3-30)[A13Aib; H18K; N24E],
SEQ ID NO: 4
EGTFISDYSIAibLDKIKQQDFVEWLLAQKPSSGAPPPS;
GIP(3-30)[A13Aib; M14L; H18K; N24E],
SEQ ID NO: 5
EGTFISDYSIAibNleDKIKQQDFVEWLLAQKPSSGAPPPS;
GIP(3-30)[A13Aib; M14Nle; H18K; N24E],
SEQ ID NO: 6
EGTFISEYSIAibLEKIKQQEFVEWLLAQKPSSGAPPPS;
GIP(3-30)[D9E; A13Aib; M14L; D15E; H18K; D21E;
N24E],
SEQ ID NO: 7
EGTFISEYSIAibNleEKIKQQEFVEWLLAQKPSSGAPPPS;
GIP(3-30)[D9E; A13Aib; M14Nle; D15E; H18K; D21E;
N24E],
SEQ ID NO: 8
EGTFISEYSIALEKIKQQEFVEWLLAQKPSSGAPPPS;
GIP(3-30)[D9E; M14L; D15E; H18K; D21E; N24E],
SEQ ID NO: 9
EGTFISEYSIANleEKIKQQEFVEWLLAQKPSSGAPPPS;
GIP(3-30)[D9E; M14Nle; D15E; H18K; D21E; N24E],
SEQ ID NO: 10
EGTFISEYSIAibLEKIKQQDFVEWLLAQKPSSGAPPPS;
GIP(3-30)[D9E; A13Aib; M14L; D15E; H18K; N24E]
SEQ ID NO: 11
XGTFISEYSIAibLEKIKQQEFVEWLLAQKPSSGAPPPS;
GIP(3-30)[E3Glutaric acid(X); D9E; A13Aib;
M14L; D15E; H18K; D21E; N24E],
SEQ ID NO: 12
XGTFISEYSIAibNleEKIKQQEFVEWLLAQKPSSGAPPPS;
GIP(3-30)[E3Glutaric acid(X); D9E; A13Aib;
M14Nle; D15E; H18K; D21E; N24E],
SEQ ID NO: 13
XGTFISEYSIALEKIKQQEFVEWLLAQKPSSGAPPPS;
GIP(3-30)[E3Glutaric acid(X); D9E; M14L; D15E;
H18K; D21E; N24E],
SEQ ID NO: 14
XGTFISEYSIAibMEKIKQQEFVEWLLAQKPSSGAPPPS;
GIP(3-30)[E3Glutaric acid(X); D9E; A13Aib; D15E;
H18K; D21E; N24E],
SEQ ID NO: 15
EGTFISEYSIAibMEKIKQQEFVEWLLAQKPSSGAPPPS;
GIP(3-30)[D9E; A13Aib; D15E; H18K; D21E; N24E],
SEQ ID NO: 16
EGTFISEYSIAMEKIKQQEFVEWLLAQKPSSGAPPPS;
GIP(3-30)[D9E; D15E; H18K; D21E; N24E],
SEQ ID NO: 17
EGTFISEYSIAibLDKIKQQDFVEWLLAQKPSSGAPPPS;
GIP(3-30)[D9E; A13Aib; M14L; H18K; N24E]
SEQ ID NO: 18
XGTFISDYSIAibMDKIKQQDFVEWLLAQKPSSGAPPPS;
GIP(3-30)[E3Glutaric acid(X); A13Aib; H18K; N24E],
SEQ ID NO: 2
EGTFISEYSIAibMEKIKQQDFVEWLLAQKPSSGAPPPS;
GIP(3-30)[D9E,A13Aib; D15E; H18K; N24E],
SEQ ID NO: 20
XGTFISEYSIAibLEKIKQQEFVEWLLAQKPSSGAPPPS;
GIP(3-30)[E3Succinic acid(X); D9E; A13Aib;
M14L; D15E; H18K; D21E; N24E],
and
SEQ ID NO: 21
XGTFISEYSIAibLEKIKQQEFVEWLLAQKPSSGAPPPS;
GIP(3-30)[E3Adipic acid(X); D9E; A13Aib; M14L;
D15E; H18K; D21E; N24E],
or a functional variant thereof comprising 1, 2 or 3 individual amino acid substitutions.

28. The peptide for use according to any one of the preceding claims, wherein the GIPR antagonist GIP peptide is selected from the group consisting of

| | |
|---|---|
| i. | EGTFISEYSIAibLEKIKQQEFVEWLLAQKPSSGAPPPS; |
| | SEQ ID NO: 6; GIP(3-30) + Cex(31-39) [D9E; |
| | A13Aib; M14L; D15E; H18K; D21E; N24E], |
| ii. | XGTFISEYSIAibNleEKIKQQEFVEWLLAQKPSSGAPPPS; |
| | SEQ ID NO: 12; GIP(3-30) + Cex(31-39) |
| | [E3Glutaric acid(X); D9E; A13Aib; M14Nle; |
| | D15E; H18K; D21E; N24E], |

and

| | |
|---|---|
| iii. | XGTFISEYSIAibLEKIKQQEFVEWLLAQKPSSGAPPPS; |
| | GIP(3-30) + Cex(31-39) [E3Glutaric |
| | acid(X); D9E; A13Aib; M14L; D15E; H18K; |
| | D21E; N24E], |

or a functional variant thereof comprising 1, 2 or 3 individual amino acid substitutions.

29. The peptide for use according to any one of the preceding claims, wherein the GIPR antagonist GIP peptide is C-terminally carboxylated (—COOH).

30. The peptide for use according to any one of the preceding claims, wherein the fatty acid molecule is a straight-chain fatty acid or a branched fatty acid.

31. The peptide for use according to any one of the preceding claims, wherein the fatty acid molecule is a diacyl fatty acid molecule.

32. The peptide for use according to any of the preceding claims, wherein said fatty acid molecule comprises an acyl group of the formula CH₃(CH₂)ₙCO—, wherein n is in an integer from 4 to 24.

33. The peptide for use according to any of the preceding claims, wherein said fatty acid molecule comprises one or more acyl groups selected from the group consisting of CH₃(CH₂)₆CO—, CH₃(CH₂)₈CO—, CH₃(CH₂)₁₀CO—, CH₃(CH₂)₁₂CO—, CH₃(CH₂)₁₄CO—, CH₃(CH₂)₁₆CO—, CH₃(CH₂)₁₈CO—, CH₃(CH₂)₂₀CO— and CH₃(CH₂)₂₂CO—.

34. The peptide for use according to any of the preceding claims, wherein said fatty acid molecule comprises an acyl group selected from the group consisting of CH₃(CH₂)₁₀CO— (lauryl, C12), CH₃(CH₂)₁₂CO— (myristoyl, C14), CH₃(CH₂)₁₄CO— (palmitoyl, C16), CH₃(CH₂)₁₆CO— (stearyl, C18), CH₃(CH₂)₁₈CO— (arachidyl, C20) and CH₃(CH₂)₂₀CO— (behenyl, C22).

35. The peptide for use according to any of the preceding claims, wherein said fatty acid molecule comprises two acyl groups individually selected from the group consisting of HOOC—CH₃(CH₂)₁₀CO— (dodecanoyl, C12), HOOC—CH₃(CH₂)₁₂CO— (1-tetradecanoyl, C14), HOOC—CH₃(CH₂)₁₄CO-(hexadecanoyl, C16), HOOC—CH₃(CH₂)₁₅CO— (15-carboxy-pentadecanoyl, C17), HOOC—CH₃(CH₂)₁₆CO— (octadecanoyl, C18), HOOC—CH₃(CH₂)₁₇CO-(17-carboxy-heptadecanoyl, C19), HOOC—CH₃(CH₂)₁₈CO— (eicosanoyl, C20), HOOC—CH₃(CH₂)₁₉CO— (19-carboxy-nonadecanoyl, C21) and HOOC—CH₃(CH₂)₂₀CO— (behenyl, C22).

36. The peptide for use according to any of the preceding claims, wherein said fatty acid molecule comprises an acyl group of the formula COOH(CH₂)ₙCO— (dicarboxylic acid), wherein n is an integer from 4 to 24.

37. The peptide for use according to any of the preceding claims, wherein said fatty acid molecule comprises an acyl group selected from the group consisting of COOH(CH₂)₁₄CO—, COOH(CH₂)₁₆CO—, COOH(CH₂)₁₈CO— and COOH(CH₂)₂₀CO—.

38. The peptide for use according to any of the preceding claims, wherein said fatty acid molecule comprises or consists of COOH(CH₂)₁₄CO—.

39. The peptide for use according to any of the preceding claims, wherein said fatty acid molecule comprises or consists of COOH(CH₂)₁₆CO—.

40. The peptide for use according to any of the preceding claims, wherein said fatty acid molecule comprises or consists of COOH(CH₂)₁₈CO—.

41. The peptide for use according to any of the preceding claims, wherein said fatty acid molecule is attached to the side chain amino group of the lysine residue at position 18 of SEQ ID NO:1, or a variant of SEQ ID NO:1.

42. The peptide for use according to any of the preceding claims, wherein said fatty acid molecule is attached to the side chain amino group of an ornithine residue at position 18 of a variant of SEQ ID NO:1.

43. The peptide for use according to any of the preceding claims, wherein said fatty acid molecule is attached to the side chain amino group of the amino acid residue at position 18 of SEQ ID NO:1, or a variant of SEQ ID NO:1, via a linker.

44. The peptide for use according to any of the preceding claims, wherein the fatty acid molecule is attached to an amino acid residue of SEQ ID NO:1, or a variant thereof, via a linker in such a way that a carboxyl group of the fatty acid molecule forms an amide bond with an amino group of the linker.

45. The peptide for use according to any of the preceding claims, wherein said linker comprises one or more moieties individually selected from the group consisting of:
a. α-amino acid, γ-amino acid or w-amino acid,
b. one or more amino acids selected from the group consisting of succinic acid, Lys, Glu, Asp,
c. one or more amino acids selected from the group consisting of Gly and Ser,
d. one or more amino acids selected from the group consisting of Ala, Glu, Lys and Leu,
e. one or more of γ-aminobutanoyl (γ-aminobutyric acid), γ-Glu (γ-glutamic acid), β-Asp (β-asparagyl), β-Ala (β-alanyl), 2-aminoisobutyric acid (Aib) and Gly, and
f. [8-amino-3,6-dioxaoctanoic acid]n (AEEAcₙ), wherein n is an integer between 1 and 50, such as an integer between 1-4, 1-3 or 1-2.

46. The peptide for use according to any one of the preceding claims, wherein said linker comprises a γ-Glu, one or more 8-amino-3,6-dioxaoctanoic acid (AEEAc), or combinations thereof, for example wherein said linker comprises or consists of a [8-amino-3,6-dioxaoctanoic acid]ₙ (AEEAc)ₙ, wherein n is an integer between 1 and 50, such as an integer between 1-2, 2-3, 3-4, 4-5, 5-6, 6-7, 7-8, 8-9, 9-10, 10-11, 11-12, 12-13, 13-14, 14-15, 15-20, 20-25, 25-30, 30-35, 35-40, 40-45, 45-50, preferably wherein n is 1, 2 or 3.

47. The peptide for use according to any one of the preceding claims, wherein said linker comprises or consists of a γ-Glu and two AEEAc, such as wherein said linker comprises or consists of γ-Glu-AEEAc-AEEAc.

48. The peptide for use according to any one of the preceding claims, wherein the fatty acid molecule is attached to the side chain amino group of the amino acid residue at position 18 of SEQ ID NO:1, or said functional variant thereof, via a linker, and wherein the combination of linker and fatty acid molecule is selected from the group consisting of:
i. Hexadecanoyl-γ-Glu-
ii. Hexadecanoyl-γ-Glu-γ-Glu-
iii. Hexadecanoyl-γ-Glu-AEEAc-
iv. Hexadecanoyl-γ-Glu-AEEAc-AEEAc-
v. Hexadecanoyl-γ-Glu-AEEAc-AEEAc-AEEAc-
vi. [15-carboxy-pentadecanoyl]-γ-Glu-
vii. [15-carboxy-pentadecanoyl]-γ-Glu-γ-Glu-
viii. [15-carboxy-pentadecanoyl]-γ-Glu-AEEAc-
ix. [15-carboxy-pentadecanoyl]-γ-Glu-AEEAc-AEEAc-
x. [15-carboxy-pentadecanoyl]-γ-Glu-AEEAc-AEEAc-AEEAc-
xi. Octadecanoyl-γ-Glu-
xii. Octadecanoyl-γ-Glu-γ-Glu-
xiii. Octadecanoyl-γ-Glu-AEEAc-
xiv. Octadecanoyl-γ-Glu-AEEAc-AEEAc-
xv. Octadecanoyl-γ-Glu-AEEAc-AEEAc-AEEAc-
xvi. [17-carboxy-heptadecanoyl]-γ-Glu-
xvii. [17-carboxy-heptadecanoyl]-γ-Glu-γ-Glu-
xviii. [17-carboxy-heptadecanoyl]-γ-Glu-AEEAc-
xix. [17-carboxy-heptadecanoyl]-γ-Glu-AEEAc-AEEAc-
xx. [17-carboxy-heptadecanoyl]-γ-Glu-AEEAc-AEEAc-AEEAc-
xxi. Eicosanoyl-γ-Glu-
xxii. Eicosanoyl-γ-Glu-γ-Glu-
xxiii. Eicosanoyl-γ-Glu-AEEAc-
xxiv. Eicosanoyl-γ-Glu-AEEAc-AEEAc-
xxv. Eicosanoyl-γ-Glu-AEEAc-AEEAc-AEEAc-
xxvi. [19-carboxy-nonadecanoyl]-γ-Glu-
xxvii. [19-carboxy-nonadecanoyl]-γ-Glu-γ-Glu-
xxviii. [19-carboxy-nonadecanoyl]-γ-Glu-AEEAc-
xxix. [19-carboxy-nonadecanoyl]-γ-Glu-AEEAc-AEEAc-, and
xxx. [19-carboxy-nonadecanoyl]-γ-Glu-AEEAc-AEEAc-AEEAc-.

49. The peptide for use according to any one of the preceding claims, wherein the fatty acid molecule is attached to the side chain amino group of the amino acid residue at position 18 of SEQ ID NO:1, or said functional variant thereof, via a linker, and wherein the combination of linker and fatty acid molecule is [17-carboxy-heptadecanoyl]-γ-Glu-AEEAc-AEEAc-.

50. The peptide for use according to any one of the preceding claims, wherein the fatty acid molecule is attached to the side chain amino group of the amino acid residue at position 18 of SEQ ID NO:1, or said functional variant thereof, and wherein the fatty acid is selected from the group consisting of:
i. [15-Carboxy pentadecanoyl], and
ii. [17-carboxy-heptadecanoyl].

51. The peptide for use according to any one of the preceding claims, wherein the GIPR antagonist GIP peptide is selected from the group consisting of:
SEQ ID NO: 38
EGTFISEYSIAMEKIKQQDFVEWLLAQKPSSGAPPPS-C16-diacid/18K;
GIP(3-30) + Cex(31-39)[D9E; D15E; H18K; N24E],
SEQ ID NO: 1
EGTFISEYSAibANleEKIKQQDFVEWLLAQKPSSGAPPPS-C16-diacid/18K;
GIP(3-30) + Cex(31-39)
[D9E; I12Aib; M14Nle; D15E; H18K; N24E],
SEQ ID NO: 2
EGTFISEYSIAibMEKIKQQDFVEWLLAQKPSSGAPPPS-C16-diacid/18K;
GIP(3-30) + Cex(31-39)[D9E; A13Aib; D15E; H18K; N24E],
SEQ ID NO: 3
EGTFISDYSIAibMDKIKQQDFVEWLLAQKPSSGAPPPS-C16-diacid/18K;
GIP(3-30) + Cex(31-39)[A13Aib; H18K; N24E],
SEQ ID NO: 4
EGTFISDYSIAibLDKIKQQDFVEWLLAQKPSSGAPPPS-C16-diacid/18K;
GIP(3-30) + Cex(31-39)[A13Aib; M14L; H18K; N24E],
SEQ ID NO: 4
EGTFISDYSIAibLDKIKQQDFVEWLLAQKPSSGAPPPS-2xAEEAc + yGlu-
C18-diacid/18K; GIP(3-30) + Cex(31-39)
[A13Aib; M14L; H18K; N24E],
SEQ ID NO: 5
EGTFISDYSIAibNleDKIKQQDFVEWLLAQKPSSGAPPPS-C16-diacid/18K;
GIP(3-30) + Cex(31-39)[A13Aib; M14Nle; H18K; N24E],
SEQ ID NO: 5
EGTFISDYSIAibNleDKIKQQDFVEWLLAQKPSSGAPPPS-2xAEEAc + yGlu-
C18-diacid/18K; GIP(3-30) + Cex(31-39)
[A13Aib; M14Nle; H18K; N24E],
SEQ ID NO: 6
EGTFISEYSIAibLEKIKQQEFVEWLLAQKPSSGAPPPS-C16-diacid/18K;
GIP(3-30) + Cex(31-39)
[D9E; A13Aib; M14L; D15E; H18K; D21E; N24E],
SEQ ID NO: 6
EGTFISEYSIAibLEKIKQQEFVEWLLAQKPSSGAPPPS-2xAEEAc + yGlu-
C18-diacid/18K; GIP(3-30) + Cex(31-39)
[D9E; A13Aib; M14L; D15E; H18K; D21E; N24E],
SEQ ID NO: 7
EGTFISEYSIAibNleEKIKQQEFVEWLLAQKPSSGAPPPS-C16-diacid/18K;
GIP(3-30) + Cex(31-39)
[D9E; A13Aib; M14Nle; D15E; H18K; D21E; N24E],
SEQ ID NO: 7
EGTFISEYSIAibNleEKIKQQEFVEWLLAQKPSSGAPPPS-2xAEEAc + yGlu-
C18-diacid/18K; GIP(3-30) + Cex(31-39)
[D9E; A13Aib; M14Nle; D15E; H18K; D21E; N24E],
SEQ ID NO: 8
EGTFISEYSIALEKIKQQEFVEWLLAQKPSSGAPPPS-2xAEEAc + yGlu-
C18-diacid/18K; GIP(3-30) + Cex(31-39)
[D9E; M14L; D15E; H18K; D21E; N24E],
SEQ ID NO: 9
EGTFISEYSIANleEKIKQQEFVEWLLAQKPSSGAPPPS-2xAEEAc + yGlu-
C18-diacid/18K; GIP(3-30) + Cex(31-39)
[D9E; M14Nle; D15E; H18K; D21EN24E],
SEQ ID NO: 11
XGTFISEYSIAibLEKIKQQEFVEWLLAQKPSSGAPPPS-2xAEEAc + yGlu-
C18-diacid/18K; GIP(3-30) + Cex(31-39)[E3Glutaric
acid(X); D9E; A13Aib; M14L; D15E; H18K; D21E; N24E],
SEQ ID NO: 12
XGTFISEYSIAibNleEKIKQQEFVEWLLAQKPSSGAPPPS-2xAEEAc + yGlu-
C18-diacid/18K; GIP(3-30) + Cex(31-39)[E3Glutaric
acid(X); D9E; A13Aib; M14Nle; D15E; H18K; D21E; N24E],
SEQ ID NO: 13
XGTFISEYSIALEKIKQQEFVEWLLAQKPSSGAPPPS-OH-2xAEEAc + yGlu-
C18-diacid/18K; GIP(3-30) + Cex(31-39)[E3Glutaric
acid(X); D9E; M14L; D15E; H18K; D21E; N24E],
SEQ ID NO: 14
XGTFISEYSIAibMEKIKQQEFVEWLLAQKPSSGAPPPS-2xAEEAc + yGlu-
C18-diacid/18K; GIP(3-30) + Cex(31-39)[E3Glutaric
acid(X); D9E; A13Aib; D15E; H18K; D21E; N24E],
SEQ ID NO: 15
EGTFISEYSIAibMEKIKQQEFVEWLLAQKPSSGAPPPS-2xAEEAc + yGlu-
C18-diacid/18K; GIP(3-30) + Cex(31-39)
[D9E; A13Aib; D15E; H18K; D21E; N24E],
SEQ ID NO: 16
EGTFISEYSIAMEKIKQQEFVEWLLAQKPSSGAPPPS-2xAEEAc + yGlu-
C18-diacid/18K; GIP(3-30) + Cex(31-39)
[D9E; D15E; H18K; D21E; N24E],
SEQ ID NO: 17
EGTFISEYSIAibLDKIKQQDFVEWLLAQKPSSGAPPPS-2xAEEAc + yGlu-
C18-diacid/18K; GIP(3-30) + Cex(31-39)[D9E; A13Aib;
M14L; H18K; N24E],
SEQ ID NO: 11
XGTFISEYSIAibLEKIKQQEFVEWLLAQKPSSGAPPPS-2xAEEAc + yGlu-
C18-diacid/18K; GIP(3-30) + Cex(31-39)[E3Glutaric
acid(X); D9E; A13Aib; M14L; D15E; H18K; D21E; N24E],
SEQ ID NO: 6
EGTFISEYSIAibLEKIKQQEFVEWLLAQKPSSGAPPPS-2xAEEAc + yGlu-
C18-diacid/18K; GIP(3-30) + Cex(31-39)
[D9E; A13Aib; M14L; D15E; H18K; D21E; N24E],
SEQ ID NO: 18
XGTFISDYSIAibMDKIKQQDFVEWLLAQKPSSGAPPPS-C16-diacid/18K;
GIP(3-30) + Cex(31-39)[E3Glutaric
acid(X); A13Aib; H18K; N24E],
SEQ ID NO: 2
EGTFISEYSIAibMEKIKQQDFVEWLLAQKPSSGAPPPS-C16-diacid/18K;
GIP(3-30) + Cex(31-39)[D9E; A13Aib; D15E; H18K; N24E],
SEQ ID NO: 11
XGTFISEYSIAibLEKIKQQEFVEWLLAQKPSSGAPPPS-C16-diacid/18K;
GIP(3-30) + Cex(31-39)[E3Glutaric
acid(X); D9E; A13Aib; M14L; D15E; H18K; D21E; N24E],
SEQ ID NO: 8
EGTFISEYSIALEKIKQQEFVEWLLAQKPSSGAPPPS-C16-diacid/18K;
GIP(3-30) + Cex(31-39)[D9E; M14L; D15E; H18K; N24E],
SEQ ID NO: 6
EGTFISEYSIAibLEKIKQQEFVEWLLAQKPSSGAPPPS-(GGGS-C16-
diacid/18K); GIP(3-30) + Cex(31-39)
[D9E; A13Aib; M14L; D15E; H18K; D21E; N24E],
SEQ ID NO: 6
EGTFISEYSIAibLEKIKQQEFVEWLLAQKPSSGAPPPS-(ALEA-C16-
diacid/18K); GIP(3-30) + Cex(31-39)
[D9E; A13Aib; M14L; D15E; H18K; D21E; N24E],
SEQ ID NO: 10
EGTFISEYSIAibLEKIKQQDFVEWLLAQKPSSGAPPPS-2xAEEAc + yGlu-
C18-diacid/18K; GIP(3-30) + Cex(31-39)[D9E; A13Aib;
M14L; D15E; H18K; N24E],
SEQ ID NO: 12
XGTFISEYSIAibNleEKIKQQEFVEWLLAQKPSSGAPPPS-C16-diacid/18K);
GIP(3-30) + Cex(31-39)[E3Glutaric
acid(X); D9E; A13Aib; M14Nle; D15E; H18K; D21E; N24E],
SEQ ID NO: 6
EGTFISEYSIAibLEKIKQQEFVEWLLAQKPSSGAPPPS-(Aib-C16-
diacid/18K); GIP(3-30) + Cex(31-39)
[D9E; A13Aib; M14L; D15E; H18K; D21E; N24E],
SEQ ID NO: 6
EGTFISEYSIAibLEKIKQQEFVEWLLAQKPSSGAPPPS-
(KAAAEKAAAEKAAAE-C16-diacid/18K); GIP(3-30) + Cex(31-39)
[D9E; A13Aib; M14L; D15E; H18K; D21E; N24E],
SEQ ID NO: 20
XGTFISEYSIAibLEKIKQQEFVEWLLAQKPSSGAPPPS-2xAEEAc + yGlu-
C18-diacid/18K; GIP(3-30) + Cex(31-39)[E3Succinic
acid(X); D9E; A13Aib; M14L; D15E; H18K; D21E; N24E],
and
SEQ ID NO: 21
XGTFISEYSIAibLEKIKQQEFVEWLLAQKPSSGAPPPS-2xAEEAc + yGlu-
C18-diacid/18K; GIP(3-30) + Cex(31-39)[E3Adipic
acid(X); D9E; A13Aib; M14L; D15E; H18K; D21E; N24E],
or a functional variant thereof comprising 1, 2 or 3 individual amino acid substitutions.

52. The peptide for use according to any one of the preceding claims, wherein the GIPR antagonist GIP peptide is selected from the group consisting of:
SEQ ID NO: 6

| | |
|---|---|
| i. | EGTFISEYSIAibLEKIKQQEFVEWLLAQKPSSGAPPPS-C16- |
| | diacid/18K; GIP(3-30) + Cex(31-39) |
| | [D9E; A13Aib; M14L; D15E; H18K; D21E; N24E], |

SEQ ID NO: 12

| | |
|---|---|
| ii. | XGTFISEYSIAibNleEKIKQQEFVEWLLAQKPSSGAPPPS- |
| | 2xAEEAc + yGlu-C18-diacid/18K; GIP(3-30) + |
| | Cex(31-39)[E3Glutaric acid(X); D9E; A13Aib; |
| | M14Nle; D15E; H18K; D21E; N24E], |

and
SEQ ID NO: 11

| | |
|---|---|
| iii. | XGTFISEYSIAibLEKIKQQEFVEWLLAQKPSSGAPPPS- |
| | C16-diacid/18K; GIP(3-30) + Cex(31-39) |
| | [E3Glutaric acid(X); D9E; A13Aib; M14L; |
| | D15E; H18K; D21E; N24E], |

or a functional variant thereof comprising 1, 2 or 3 individual amino acid substitutions.

53. The peptide for use according to any one of the preceding claims, wherein the GIPR antagonist GIP peptide is EGTFISEYSIAibLEKIKQQEFVEWLLAQKPSSGAPPPS-C16-diacid/18K; SEQ ID NO:6; GIP(3-30)+Cex(31-39) [D9E;A13Aib;M14L;D15E;H18K;D21E;N24E] or a functional variant thereof comprising 1, 2 or 3 individual amino acid substitutions.

54. The peptide for use according to any one of the preceding claims, wherein the GIPR antagonist GIP peptide is XGTFISEYSIAibNIeEKIKQQEFVEWLLAQKPSSGAPPPS-2xAEEAc+yGlu-C18-diacid/18K; SEQ ID NO:12; GIP(3-30)+Cex(31-39) [E3Glutaric acid(X);D9E;A13Aib; M14Nle;D15E;H18K;D21E;N24E] or a functional variant thereof comprising 1, 2 or 3 individual amino acid substitutions.

55. The peptide for use according to any one of the preceding claims, wherein the GIPR antagonist GIP peptide is XGTFISEYSIAibLEKIKQQEFVEWLLAQKPSSGAPPPS-C16-diacid/18K; SEQ ID NO:11; GIP(3-30)+Cex(31-39) [E3Glutaric acid(X);D9E;A13Aib;M14L;D15E;H18K;D21E;N24E] or a functional variant thereof comprising 1, 2 or 3 individual amino acid substitutions.

56. The peptide for use according to any one of the preceding claims, wherein the GIP peptide and/or the GLP-1 receptor agonist are administered at least once daily, such as once daily, such as once each second day, such as once each third day, such as once each fourth day, such as once each fifth day, such as once each sixth day, such as once weekly, such as once each second week, such as once monthly.

57. The peptide for use according to any one of the preceding claims, wherein said GIPR antagonist GIP peptide is administered once daily.

58. The peptide for use according to any one of the preceding claims, wherein said GIPR antagonist GIP peptide is administered once daily.

59. The peptide for use according to any one of the preceding claims, wherein said GIPR antagonist GIP peptide is administered once weekly.

60. The peptide for use according to any one of the preceding claims, wherein said GIPR antagonist GIP peptide is administered at a dosage of about 15 nmol/kg, such as about 20 nmol/kg, such as about 25 nmol/kg, such as about 30 nmol/kg, such as about 35 nmol/kg, such as about 40 nmol/kg, such as about 45 nmol/kg, such as about 50 nmol/kg, such as about 55 nmol/kg, such as about 60 nmol/kg, such as about 65 nmol/kg, such as about 70 nmol/kg, such as about 75 nmol/kg, such as about 80 nmol/kg, such as about 85 nmol/kg, such as about 90 nmol/kg, such as about 95 nmol/kg, such as about 100 nmol/kg, such as about 110 nmol/kg, such as about 120 nmol/kg, such as about 125 nmol/kg, such as about 150 nmol/kg, such as about 175 nmol/kg, such as about 200 nmol/kg, such as about 250 nmol/kg, such as about 300 nmol/kg, such as about 350 nmol/kg, such as about 400 nmol/kg, such as about 450 nmol/kg, such as about 500 nmol/kg, such as about 550 nmol/kg, such as about 600 nmol/kg, such as about 650 nmol/kg, such as about 700 nmol/kg, such as about 750 nmol/kg, such as about 800 nmol/kg, such as about 850 nmol/kg, such as about 900 nmol/kg, such as about 950 nmol/kg, such as about 1000 nmol/kg, such as about 1050 nmol/kg, such as about 1100 nmol/kg, such as about 1150 nmol/kg, such as about 1200 nmol/kg, such as about 1250 nmol/kg, such as about 1300 nmol/kg, such as about 1350 nmol/kg, such as about 1400 nmol/kg, such as about 1450 nmol/kg, such as about 1500 nmol/kg, such as about 2000 nmol/kg, such as about 2500 nmol/kg.

61. The peptide for use according to any one of the preceding claims, wherein said GLP-1 receptor agonist is administered once daily.

62. The peptide for use according to any one of the preceding claims, wherein said GLP-1 receptor agonist is administered once weekly.

63. The peptide for use according to any one of the preceding claims, wherein said GLP-1 receptor agonist is administered at a dosage of about 5 μg/kg, such as about 10 μg/kg, such as about 15 μg/kg, such as about 20 μg/kg, such as about 25 μg/kg, such as about 30 μg/kg, such as about 40 μg/kg, such as about 50 μg/kg, such as about 75 μg/kg, such as about 100 μg/kg, such as about 200 μg/kg, such as about 300 μg/kg, such as about 400 μg/kg, such as about 500 μg/kg, such as about 600 μg/kg, such as about 700 μg/kg, such as about 800 μg/kg, such as about 900 μg/kg, such as about 1 mg/kg, such as about 5 mg/kg, such as about 10 mg/kg, such as about 25 mg/kg, such as about 50 mg/kg, such as about 100 mg/kg.

64. The peptide for use according to any one of the preceding claims, wherein the GIP peptide and/or the GLP-1 receptor agonist are administered systemically.

65. The peptide for use according to any one of the preceding claims, wherein the GIP peptide and/or the GLP-1 receptor agonist are administered by infusion.

66. The peptide for use according to any one of the preceding claims, wherein the GIP peptide and/or the GLP-1 receptor agonist are administered by injection.

67. The peptide for use according to any one of the preceding claims, wherein the GIP peptide and/or the GLP-1 receptor agonist are administered parenterally, including subcutaneous, intramuscular, intrathecal, intracerebral, intravenous and intradermal administration.

68. The peptide for use according to any one of the preceding claims, wherein the GIP peptide and/or the GLP-1 receptor agonist are administered subcutaneously.

69. A composition comprising, separately or together, or a kit of parts comprising, a GIPR antagonist GIP peptide and a GLP-1 receptor agonist, wherein said GIPR antagonist GIP peptide consist of amino acid sequence SEQ ID NO:1:
(SEQ ID NO: 1)
3 - 4 - 5 - 6 7 8 9 10 11 12 13
X₁ - G - T - F - I - S - E - Y - S - I - Aib -
14 15 16 17 18 19 20 21 22 23 24 25
X₂ - E - K - I - K - Q - Q - E - F - V - E - W -
26 27 28 29 30 31 32 33 34 35 36 37
L - L - A - Q - K - P - S - S - G - A - P - P -
38 39
P - S,
wherein X₁ is E, glutaric acid, adipic acid or succinic acid;
wherein X₂ is M, L or Nle;
or a functional variant thereof, wherein the amino acid sequence of said variant differs from SEQ ID NO:1 in that the amino acid sequence of the variant comprises 1, 2 or 3 individual amino acid substitutions, wherein said peptide comprises a fatty acid molecule attached to the side chain amino group of the amino acid residue at position 18 of SEQ ID NO:1, or said functional variant thereof.

70. The composition or kit of parts according to claim 69 for use in a method of treating obesity, obesity-related disorders and/or dyslipidemia.

71. The composition or kit of parts according to claim 69 for use in a method of inhibiting or reducing one or more of i) food intake, ii) body weight, iii) fasting blood levels of glucose, iv) fasting blood levels of insulin, v) insulin resistance, vi) fasting blood levels of triglycerides, vii) fasting blood levels of cholesterol and viii) fasting blood levels of low-density lipoprotein (LDL).

72. The composition or kit of parts for use according to any one of claims 69 to 71, said method comprising one or more steps of co-administering a GIPR antagonist GIP peptide and a GLP-1 receptor agonist; and/or one or more steps of administering a GIPR antagonist GIP peptide and a GLP-1 receptor agonist.

73. The peptide or composition for use according to any one of the preceding claims, wherein said obesity-related disorder occurs in an individual with a BMI of >18.

74. The peptide or composition for use according to any one of the preceding claims, wherein said obesity-related disorder occurs in an individual with a BMI of 18 to 25.

75. The peptide or composition for use according to any one of the preceding claims, wherein said obesity-related disorder occurs in an individual with a BMI of >25.

76. The peptide or composition for use according to any one of the preceding claims, wherein said obesity-related disorder occurs in an individual with a BMI of 30 to <35.

77. The peptide or composition for use according to any one of the preceding claims, wherein said obesity-related disorder occurs in an individual with a BMI of 35 to <40.

78. The peptide or composition for use according to any one of the preceding claims, wherein said obesity-related disorder occurs in an individual with a BMI of >40.

79. The peptide or composition for use according to any one of the preceding claims, wherein said obesity is defined as an individual with a BMI of >25 and having one or more obesity-related disorders.

80. The peptide or composition for use according to any one of the preceding claims, wherein said obesity is defined as an individual with a BMI of 30 to <35.

81. The peptide or composition for use according to any one of the preceding claims, wherein said obesity is defined as an individual with a BMI of 35 to <40.

82. The peptide or composition for use according to any one of the preceding claims, wherein said obesity is defined as an individual with a BMI of >40.

83. The peptide or composition for use according to any one of the preceding claims, wherein said obesity-related disorder is binge eating.

84. The peptide or composition for use according to any one of the preceding claims, wherein said obesity-related disorder is selected from the group consisting of heart disease, stroke, high blood pressure, high blood cholesterol, high blood low-density lipoprotein (LDL) cholesterol, high blood triglycerides, dyslipidemia, gallbladder disease and gallstones, NAFLD (non-alcoholic fatty liver disease) and NASH (non-alcoholic steatohepatitis), osteoarthritis, gout, breathing problems such as sleep apnea and asthma, insulin resistance and diabetes mellitus.

85. The peptide or composition for use according to any one of the preceding claims, wherein said obesity-related disorder is diabetes mellitus, Type II.

86. The peptide or composition for use according to any one of the preceding claims, wherein said obesity-related disorder is dyslipidemia.

87. The peptide or composition for use according to any one of the preceding claims, wherein said GIPR antagonist GIP peptide and said GLP-1 receptor agonist are contained together in the same composition or pharmaceutical formulation.

88. The peptide or composition for use according to any one of the preceding claims, wherein said GIPR antagonist GIP peptide and said GLP-1 receptor agonist are each contained in separate compositions or pharmaceutical formulations.

89. The peptide or composition for use according to any one of the preceding claims, wherein said GIPR antagonist GIP peptide and said GLP-1 receptor agonist are administered separately, simultaneously, or sequentially.

90. The peptide or composition for use according to any one of the preceding claims, wherein said GIPR antagonist GIP peptide and said GLP-1 receptor agonist are administered separately.

91. The peptide or composition for use according to any one of the preceding claims, wherein said GIPR antagonist GIP peptide and said GLP-1 receptor agonist are administered simultaneously.

92. The peptide or composition for use according to any one of the preceding claims, wherein said GIPR antagonist GIP peptide and said GLP-1 receptor agonist are administered sequentially.

93. The peptide or composition for use according to any one of the preceding claims, or the composition or kit of parts according to any one of claims 69 to 72, wherein said GIPR antagonist GIP peptide and said GLP-1 receptor agonist is as specified in any one of claims 1 to 68.

94. The peptide or composition for use according to any one of the preceding claims, wherein said GIP peptide is selected from the group consisting of: EGTFISEYSIAibLEKIKQQEFVEWLLAQKPSSGAPPPS; SEQ ID NO:6; GIP(3-30) [D9E;A13Aib;M14L;D15E;H18K;D21E;N24E], XGTFISEYSIAibLEKIKQQEFVEWLLAQKPSSGAPPPS; SEQ ID NO:11; GIP(3-30) [E3Glutaric acid(X);D9E;A13Aib;M14L;D15E;H18K;D21E;N24E], and XGTFISEYSIAibNIeEKIKQQEFVEWLLAQKPSSGAPPPS; SEQ ID NO:12; GIP(3-30) [E3Glutaric acid(X);D9E;A13Aib;M14Nle;D15E;H18K;D21E;N24E], or a functional variant thereof comprising 1, 2 or 3 individual amino acid substitutions; and wherein the GLP-1 receptor agonist is (Arg34)hGLP-1(7-37) (SEQ ID NO:25), or a variant thereof, such as variant having one individual amino acid substitution, and optionally comprising a fatty acid molecule.

95. The peptide or composition for use according to any one of the preceding claims, wherein said GIP peptide is selected from the group consisting of: EGTFISEYSIAibLEKIKQQEFVEWLLAQKPSSGAPPPS; SEQ ID NO:6; GIP(3-30) [D9E;A13Aib;M14L;D15E;H18K;D21E;N24E], XGTFISEYSIAibLEKIKQQEFVEWLLAQKPSSGAPPPS; SEQ ID NO:11; GIP(3-30) [E3Glutaric acid(X);D9E;A13Aib;M14L;D15E;H18K;D21E;N24E], and XGTFISEYSIAibNIeEKIKQQEFVEWLLAQKPSSGAPPPS; SEQ ID NO:12; GIP(3-30) [E3Glutaric acid(X);D9E;A13Aib;M14Nle;D15E;H18K;D21E;N24E], or a functional variant thereof comprising 1, 2 or 3 individual amino acid substitutions; and wherein the GLP-1 receptor agonist is (Arg34)hGLP-1(7-37) (SEQ ID NO:25) comprising a fatty acid molecule attached to the lysine at position 26 of said SEQ ID NO:25, or a variant thereof, such as a variant having one individual amino acid substitution.

96. The peptide or composition for use according to any one of the preceding claims, wherein said GIP peptide is selected from the group consisting of: EGTFISEYSIAibLEKIKQQEFVEWLLAQKPSSGAPPPS; SEQ ID NO:6; GIP(3-30) [D9E;A13Aib;M14L;D15E;H18K;D21E;N24E], XGTFISEYSIAibLEKIKQQEFVEWLLAQKPSSGAPPPS; SEQ ID NO:11; GIP(3-30) [E3Glutaric acid(X);D9E;A13Aib;M14L;D15E;H18K;D21E;N24E], and XGTFISEYSIAibNIeEKIKQQEFVEWLLAQKPSSGAPPPS; SEQ ID NO:12; GIP(3-30) [E3Glutaric acid(X);D9E;A13Aib;M14Nle;D15E;H18K;D21E;N24E], or a functional variant thereof comprising 1, 2 or 3 individual amino acid substitutions; and wherein the GLP-1 receptor agonist is liraglutide or semaglutide.

97. The peptide or composition for use according to any one of the preceding claims, wherein said GIP peptide is selected from the group consisting of: EGTFISEYSIAibLEKIKQQEFVEWLLAQKPSSGAPPPS; SEQ ID NO:6; GIP(3-30) [D9E;A13Aib;M14L;D15E;H18K;D21E;N24E], XGTFISEYSIAibLEKIKQQEFVEWLLAQKPSSGAPPPS; SEQ ID NO:11; GIP(3-30) [E3Glutaric acid(X);D9E;A13Aib;M14L;D15E;H18K;D21E;N24E], and XGTFISEYSIAibNIeEKIKQQEFVEWLLAQKPSSGAPPPS; SEQ ID NO:12; GIP(3-30) [E3Glutaric acid(X);D9E;A13Aib;M14Nle;D15E;H18K;D21E;N24E], or a functional variant thereof comprising 1, 2 or 3 individual amino acid substitutions; and wherein the GLP-1 receptor agonist is semaglutide.

98. The peptide or composition for use according to any one of the preceding claims, wherein said GIP peptide is selected from the group consisting of: EGTFISEYSIAibLEKIKQQEFVEWLLAQKPSSGAPPPS-C16-diacid/18K; SEQ ID NO:6; GIP(3-30)+Cex(31-39) [D9E;A13Aib;M14L;D15E;H18K;D21E;N24E], XGTFISEYSIAibLEKIKQQEFVEWLLAQKPSSGAPPPS-C16-diacid/18K: SEQ ID NO:11; GIP(3-30)+Cex(31-39) [E3Glutaric acid(X);D9E;A13Aib;M14L;D15E;H18K;D21E;N24E],
and XGTFISEYSIAibNIeEKIKQQEFVEWLLAQKPSSGAPPPS-2xAEEAc+yGlu-C18-diacid/18K; SEQ ID NO:12; GIP(3-30)+Cex(31-39) [E3Glutaric acid(X);D9E;A13Aib; M14Nle;D15E;H18K;D21E;N24E], or a functional variant thereof comprising 1, 2 or 3 individual amino acid substitutions;
and wherein the GLP-1 receptor agonist is (Arg34)hGLP-1(7-37) (SEQ ID NO:25), or a variant thereof, such as variant having one individual amino acid substitution, and optionally comprising a fatty acid molecule.

99. The peptide or composition for use according to any one of the preceding claims, wherein said GIP peptide is selected from the group consisting of: EGTFISEYSIAibLEKIKQQEFVEWLLAQKPSSGAPPPS-C16-diacid/18K; SEQ ID NO:6; GIP(3-30)+Cex(31-39) [D9E;A13Aib;M14L;D15E;H18K;D21E;N24E], XGTFISEYSIAibLEKIKQQEFVEWLLAQKPSSGAPPPS-C16-diacid/18K; SEQ ID NO:11; GIP(3-30)+Cex(31-39) [E3Glutaric acid(X);D9E;A13Aib;M14L;D15E;H18K;D21E;N24E],
and XGTFISEYSIAibNIeEKIKQQEFVEWLLAQKPSSGAPPPS-2xAEEAc+yGlu-C18-diacid/18K; SEQ ID NO:12; GIP(3-30)+Cex(31-39) [E3Glutaric acid(X);D9E;A13Aib; M14Nle;D15E;H18K;D21E;N24E], or a functional variant thereof comprising 1, 2 or 3 individual amino acid substitutions;
and wherein the GLP-1 receptor agonist is (Arg34)hGLP-1(7-37) (SEQ ID NO:25) comprising a fatty acid molecule attached to the lysine at position 26 of said SEQ ID NO:25, or a variant thereof, such as a variant having one individual amino acid substitution.

100. The peptide or composition for use according to any one of the preceding claims, wherein said GIP peptide is selected from the group consisting of: EGTFISEYSIAibLEKIKQQEFVEWLLAQKPSSGAPPPS-C16-diacid/18K; SEQ ID NO:6; GIP(3-30)+Cex(31-39) [D9E;A13Aib;M14L;D15E;H18K;D21E;N24E], XGTFISEYSIAibLEKIKQQEFVEWLLAQKPSSGAPPPS-C16-diacid/18K; SEQ ID NO:11; GIP(3-30)+Cex(31-39) [E3Glutaric acid(X);D9E;A13Aib;M14L;D15E;H18K;D21E;N24E],
and XGTFISEYSIAibNIeEKIKQQEFVEWLLAQKPSSGAPPPS-2xAEEAc+yGlu-C18-diacid/18K; SEQ ID NO:12; GIP(3-30)+Cex(31-39) [E3Glutaric acid(X);D9E;A13Aib; M14Nle;D15E;H18K;D21E;N24E], or a functional variant thereof comprising 1, 2 or 3 individual amino acid substitutions;
and wherein the GLP-1 receptor agonist is liraglutide or semaglutide.

101. The peptide or composition for use according to any one of the preceding claims, wherein said GIP peptide is selected from the group consisting of: EGTFISEYSIAibLEKIKQQEFVEWLLAQKPSSGAPPPS-C16-diacid/18K; SEQ ID NO:6; GIP(3-30)+Cex(31-39) [D9E;A13Aib;M14L;D15E;H18K;D21E;N24E], XGTFISEYSIAibLEKIKQQEFVEWLLAQKPSSGAPPPS-C16-diacid/18K; SEQ ID NO:11; GIP(3-30)+Cex(31-39) [E3Glutaric acid(X);D9E;A13Aib;M14L;D15E;H18K;D21E;N24E],
and XGTFISEYSIAibNIeEKIKQQEFVEWLLAQKPSSGAPPPS-2xAEEAc+yGlu-C18-diacid/18K; SEQ ID NO:12; GIP(3-30)+Cex(31-39) [E3Glutaric acid(X);D9E;A13Aib; M14Nle;D15E;H18K;D21E;N24E], or a functional variant thereof comprising 1, 2 or 3 individual amino acid substitutions;
and wherein the GLP-1 receptor agonist is semaglutide.

102. The peptide or composition for use according to any one of the preceding claims, wherein said GIP peptide is: EGTFISEYSIAibLEKIKQQEFVEWLLAQKPSSGAPPPS-C16-diacid/18K; SEQ ID NO:6; GIP(3-30)+Cex(31-39) [D9E;A13Aib;M14L;D15E;H18K;D21E;N24E],
and wherein the GLP-1 receptor agonist is semaglutide.

103. The peptide or composition for use according to any one of the preceding claims, wherein said GIP peptide is: XGTFISEYSIAibLEKIKQQEFVEWLLAQKPSSGAPPPS-C16-diacid/18K; SEQ ID NO:11; GIP(3-30)+Cex(31-39) [E3Glutaric acid(X);D9E;A13Aib;M14L;D15E;H18K;D21E;N24E], or a functional variant thereof comprising 1, 2 or 3 individual amino acid substitutions, and wherein the GLP-1 receptor agonist is semaglutide.

104. The peptide or composition for use according to any one of the preceding claims, wherein said GIP peptide is: XGTFISEYSIAibNIeEKIKQQEFVEWLLAQKPSSGAPPPS-2xAEEAc+yGlu-C18-diacid/18K; SEQ ID NO:12; GIP(3-30)+Cex(31-39) [E3Glutaric acid(X);D9E;A13Aib; M14Nle;D15E;H18K;D21E;N24E], or a functional variant thereof comprising 1, 2 or 3 individual amino acid substitutions;
and wherein the GLP-1 receptor agonist is semaglutide.

105. A GIPR antagonist GIP peptide consisting of amino acid sequence SEQ ID NO:1:
(SEQ ID NO: 1)
3 - 4 - 5 - 6 7 8 9 10 11 12 13
X₁ - G - T - F - I - S - E - Y - S - I - Aib -
14 15 16 17 18 19 20 21 22 23 24 25
X₂ - E - K - I - K - Q - Q - E - F - V - E - W -
26 27 28 29 30 31 32 33 34 35 36 37
L - L - A - Q - K - P - S - S - G - A - P - P -
38 39
P - S,
wherein X₁ is E, glutaric acid, adipic acid or succinic acid;
wherein X₂ is M, L or Nle;
or a functional variant thereof, wherein the amino acid sequence of said variant differs from SEQ ID NO:1 in that the amino acid sequence of the variant comprises 1, 2 or 3 individual amino acid substitutions,
wherein said peptide comprises a fatty acid molecule attached to the side chain amino group of the amino acid residue at position 18 of SEQ ID NO:1, or said functional variant thereof,
for use in a method of inhibiting or reducing one or more of i) food intake, ii) body weight, iii) fasting blood levels of glucose, iv) fasting blood levels of insulin, v) insulin resistance, vi) fasting blood levels of triglycerides, vii) fasting blood levels of cholesterol and viii) fasting blood levels of low-density lipoprotein (LDL) cholesterol.

106. A glucose-dependent insulinotropic peptide receptor (GIPR) antagonist GIP peptide consisting of amino acid sequence SEQ ID NO:1:
(SEQ ID NO: 1)
3 - 4 - 5 - 6 7 8 9 10 11 12 13
X₁ - G - T - F - I - S - E - Y - S - I - Aib -
14 15 16 17 18 19 20 21 22 23 24 25
X₂ - E - K - I - K - Q - Q - E - F - V - E - W -
26 27 28 29 30 31 32 33 34 35 36 37
L - L - A - Q - K - P - S - S - G - A - P - P -
38 39
P - S,
wherein X₁ is E, glutaric acid, adipic acid or succinic acid;
wherein X₂ is M, L or Nle;
or a functional variant thereof, wherein the amino acid sequence of said variant differs from SEQ ID NO:1 in that the amino acid sequence of the variant comprises 1, 2 or 3 individual amino acid substitutions, wherein said peptide comprises a fatty acid molecule attached to the side chain amino group of the amino acid residue at position 18 of SEQ ID NO:1, or said functional variant thereof,
for use in a method of treating obesity, obesity-related disorders and/or dyslipidemia.

107. The peptide for use according to any one of claims 105 and 106, wherein the GIPR antagonist GIP peptide is selected from the group consisting of:
SEQ ID NO: 6

| | |
|---|---|
| i. | EGTFISEYSIAibLEKIKQQEFVEWLLAQKPSSGAPPPS-C16- |
| | diacid/18K; GIP(3-30) + Cex(31-39) |
| | [D9E; A13Aib; M14L; D15E; H18K; D21E; N24E], |

SEQ ID NO: 12

| | |
|---|---|
| ii. | XGTFISEYSIAibNleEKIKQQEFVEWLLAQKPSSGAPPPS- |
| | 2xAEEAc + yGlu-C18-diacid/18K; GIP(3-30) + |
| | Cex(31-39)[E3Glutaric acid(X); D9E; A13Aib; |
| | M14Nle; D15E; H18K; D21E; N24E], |

and
SEQ ID NO: 11

| | |
|---|---|
| iii. | XGTFISEYSIAibLEKIKQQEFVEWLLAQKPSSGAPPPS- |
| | C16-diacid/18K; GIP(3-30) + Cex(31-39) |
| | [E3Glutaric acid(X); D9E; A13Aib; M14L; |
| | D15E; H18K; D21E; N24E], |

or a functional variant thereof comprising 1, 2 or 3 individual amino acid substitutions.

108. The peptide for use according to any one of the claims 105 to 108 wherein the GIPR antagonist GIP peptide is
SEQ ID NO: 6
EGTFISEYSIAibLEKIKQQEFVEWLLAQKPSSGAPPPS-C16-
diacid/18K; GIP(3-30) + Cex(31-39)
[D9E; A13Aib; M14L; D15E; H18K; D21E; N24E],

109. The peptide for use according to any one of the claims 105 to 108, wherein the GIPR antagonist GIP peptide is
SEQ ID NO: 12
XGTFISEYSIAibNleEKIKQQEFVEWLLAQKPSSGAPPPS-
2xAEEAc + yGlu-C18-diacid/18K; GIP(3-30) +
Cex(31-39)[E3Glutaric acid(X); D9E; A13Aib;
M14Nle; D15E; H18K; D21E; N24E],

110. The peptide for use according to any one of claims 105 to 109, wherein the GIPR antagonist GIP peptide is
SEQ ID NO: 11
XGTFISEYSIAibLEKIKQQEFVEWLLAQKPSSGAPPPS-
C16-diacid/18K; GIP(3-30) + Cex(31-39)
[E3Glutaric acid(X); D9E; A13Aib; M14L;
D15E; H18K; D21E; N24E],

111. The peptide for use according to any one of claims 105 to 110, wherein said method comprises one or more steps of co-administering a GLP-1 receptor agonist.
